(19) United States
(12) Patent Application Publication
(10) Pub. No.: US 2023/0087622 A1
(43) Pub. Date: Mar. 23, 2023

(54) BACTERIAL ADMIXTURES

(71) Applicant: The Jackson Laboratory, Bar Harbor, ME (US)

(72) Inventors: Julia Oh, Bar Harbor, ME (US); Wei Zhou, Bar Harbor, ME (US); Michelle Spoto, Bar Harbor, ME (US)

(73) Assignee: The Jackson Laboratory, Bar Harbor, ME (US)

(21) Appl. No.: 17/795,329

(22) PCT Filed: Jan. 28, 2021
(86) PCT No.: PCT/US2021/015411

§ 371 Date: Jul. 26, 2022

Related U.S. Application Data
(60) Provisional application No. 62/967,310, filed on Jan. 29, 2020.

(51) Int. Cl. A61K 35/74; A61K 9/00; C12N 1/20

(52) U.S. Cl. CPC A61K 35/74; A61K 9/0014; C12N 1/20

(57) ABSTRACT

The present disclosure provides an admixture of Staphylococcus (S.) epidermidis cells selected from S. epidermidis Type I cells, S. epidermidis Type II cells, S. epidermidis Type III cells, S. epidermidis Type Mvb cells, and S. epidermidis Type IV cells capable of modified expression of virulence factor genes and other genes that contribute to virulence, such as nitrogen respiration genes, ureases activity genes, carbohydrate metabolism genes, iron uptake genes, sulfur metabolism genes. The present disclosure also provides methods of treating or preventing skin disease and reducing the risk of recurrent skin disease in a subject by administering the admixtures of S. epidermidis cell types.

### RELATED APPLICATIONS

This application claims the benefit under 35 U.S.C. § 119(e) of U.S. provisional application No. 62/967,310, filed Jan. 29, 2020, which is incorporated by reference herein in its entirety.

### BACKGROUND

Microbial diversity is ultimately manifested at the finest taxonomic resolution: individual strains of a microbial species can exhibit widely diverse phenotypes. For example, most Escherichia (E.) coli strains are commensals in the human gastrointestinal tract, while some strains can cause severe disease (Leimbach et al., 2013). In human skin, commensal strains of Staphylococcus (S.) epidermidis, a ubiquitous skin colonizer (Oh et al., 2014, 2016), can protect against colonization by skin pathogens (Cogen et al., 2010a, 2010b; Lai et al., 2010), modulate the immune system (Lai et al., 2009; Linehan et al., 2018; Naik et al., 2012; Scharschmidt et al., 2015), and even prevent skin cancer (Nakatsuji et al., 2018). Simultaneously, S. epidermidis is a common cause of bloodstream and indwelling medical device infection (National Nosocomial Infections Surveillance System, 2004). Moreover, many clinical isolates of S. epidermidis carry genes encoding antibiotic resistance or biofilm formation (reviewed in Otto, 2009), impeding treatment.

An understanding of strain-level diversity is complicated by the observation that each human carries a distinct collection of microbial strains, as revealed by comparative metagenomic (Lloyd-Price et al., 2017; Oh et al., 2014) and culture-based studies (Nataro and Kaper, 1998). These strains can originate via maternal transmission (Asnicar et al., 2017; Ferretti et al., 2018; Yassour et al., 2018) and can be shaped by different host-specific factors, such as disease and health status (Duvallet et al., 2017; Greenblum et al., 2015; Tett et al., 2017; Zhang and Zhao, 2016). However, subsequent diversification of strains within an individual—one's own intrinsic bacterial population diversity—is not well-understood, largely due to the limitations in the sequencing depth of metagenomic studies and sample sizes of culture-based studies.

### SUMMARY

The present disclosure is based, in part, on unexpected data showing that admixtures of Staphylococcus epidermidis (S. epidermidis) cells that include multiple strains (e.g., combinations of cells from S. epidermidis agr Type I, Type II, Type III, Type IIIb, and Type IV strains) reduces the virulence of S. epidermidis. This was unexpected for at least two reasons: (1) the existence of S. epidermidis Types IIIb and IV was previously unknown; and (2) the data suggests that the effect on virulence is simply the result of combining cells of any two or more S. epidermidis Types, independent of the particular type selected. The data herein also suggests that the presence of cells from at least two or at least three different S. epidermidis types impacts bacterial quorum sensing, resulting in modifications to the cellular processes regulated by quorum sensing, such as virulence, symbiosis conjugation, and biofilm formation.

Some aspects of the present disclosure provide a composition comprising (a) an admixture of cells that comprises at least two bacterial cell types selected from Staphylococcus (S.) epidermidis Type I cells, S. epidermidis Type II cells, S. epidermidis Type III cells, S. epidermidis Type IIIb cells, and S. epidermidis Type IV cells; and (b) a synthetic excipient. In some embodiments, the synthetic excipient is selected from the group consisting of diluents, thickeners, humectants, preservatives, neutralizers, coemulsifiers, occlusive, and fragrances.

Other aspects of the present disclosure provide a composition comprising an admixture of cells that comprises at least two bacterial cell types selected from Staphylococcus (S.) epidermidis Type I cells, S. epidermidis Type II cells, S. epidermidis Type III cells, S. epidermidis Type IIIb cells, and S. epidermidis Type IV cells, wherein at least one of the S. epidermidis cell types is genetically engineered.

Further aspects of the present disclosure provide an admixture of cells that comprises at least two cell types selected from Staphylococcus (S.) epidermidis Type I cells, S. epidermidis Type II cells, S. epidermidis Type III cells, S. epidermidis Type IIIb cells, and S. epidermidis Type IV cells, wherein the admixture is formulated with an excipient for topical delivery.

In some embodiments, cells of the admixture are collectively capable of modifying an expression level of a gene selected from nitrogen respiration genes, urease activity genes, carbohydrate metabolism genes, iron uptake genes, sulfur metabolism genes, and virulence factor genes, relative to a control.

In some embodiments, the nitrogen respiration genes are selected from S. epidermidis narT, nreC, nreB, narX, narH, narG, sumT, nasE, and nasD. In some embodiments, the urease activity genes are selected from S. epidermidis ureA, ureB, ureC, ureD, ureE, and ureF. In some embodiments, the carbohydrate metabolism genes are selected from S. epidermidis FruB, GlmS, Fba, Tkt, Tpi, Gap, GapB, Pgi, Pgk, Eno, PepcK, CitB, CitC, OgdH, Sucla2, SdhA, CitG, Mdh1, PckA, LDH, PDH, ScaD, PflB, Fdh, and BdhA. In some embodiments, the iron uptake genes are selected from S. epidermidis fecD, feuC, fecE, and yclQ. In some embodiments, the sulfur metabolism genes are selected from S. epidermidis cysH, cysJ, cysI, sirC, sat, and cysC. In some embodiments, the virulence factor genes are selected from S. epidermidis icaA, icaB, icaC, icaD, icaR, ad, atlE, ebh, ebhA, ebp, geh, gehD, hlb, lip, nuc, psmB, sdrF, sdrH, se2319, sspA, and sspB.

In some embodiments, the expression level of the gene is decreased by 1.5-fold to 12-fold.

In some embodiments, at least one of the Staphylococcus epidermidis cell types is genetically engineered.

Some aspects of the present disclosure provide a method of treating or preventing skin disease in a subject comprising administering to the subject the admixture of any one of the preceding embodiments.

In some embodiments, the skin disease is selected from folliculitis, furuncles, carbuncles, impetigo, ecthyma, cellulitis, atopic dermatitis, scabies, and folliculitis decalvans.

Other aspects of the present disclosure provide a method of reducing the risk of recurrent skin disease in a subject comprising administering to the subject the admixture of any one of the preceding claims.

In some embodiments, recurrent disease is atopic dermatitis.

In some embodiments, the administration is topical administration.

### BRIEF DESCRIPTION OF THE DRAWINGS

FIGS. 1A-1D. Phylogenetic variation of the individualized Staphylococcus epidermidis (S. epidermidis) isolates. (FIG. 1A) Two alternative scenarios of within-individual evolution. Each circle represents a cluster of isolates diverged from a single founder lineage colonizing a given host. In the first scenario (left), all isolates from a given host diverged from a single founder lineage; in the second scenario (right), isolates from each host diverged from multiple distinct founder lineages. (FIG. 1B) Core-genome phylogeny (midpoint rooted) based on 58498 core-genome SNP loci for the 1482 isolates sampled in this study and 50 previously sequenced isolates from multiple diseased and healthy individuals. Skin site of each isolate is indicated. (FIG. 1C) Individualized S. epidermidis isolates evolved from multiple founder lineages. Each founder lineage is represented by a circle and is defined as the highest node from which at least 95% of the derived isolates (i.e. tip nodes) were either found in the same subject or public strains. The size of the circle represents the number of isolates derived from that lineage. (FIG. 1D) Pairwise cophenetic distances of the 1482 isolates. Note that the distribution of ‘between-subject’ distances depends on the sample size per subject, with p0, who had the most isolates cultivated, having the largest contribution. The toeweb is highlighted to illustrate its unusual between-subject similarity.

FIGS. 2A-2B. Subject-specific transmission patterns of S. epidermidis isolates. (FIG. 2A) Proportion of sister isolates shared between two skin sites. (FIG. 2B) transmission map summarizing the BEAST analysis. Shading of the lines connecting skin sites show the posterior probability that the transmission rate between the two sites was not 0. Lines with posterior probabilities <0.3 were removed for better visualization.

FIGS. 3A-3D. Bayesian inference of evolutionary history. (FIG. 3A) Maximum clade credibility tree annotated with historical transmission events. The lines of the nodes and branches denote the predicted skin sites of the ancestral lineages, while the sizes of the nodes are proportional to the posterior probabilities of the skin site predictions. N=2000 trees were used for final transmission inference. The tree was transformed into a cladogram for better visualization. Arrows illustrate example transmission events from Nares_R to Cheek_L. (FIG. 3B) Estimated chronological ages of all ancestral nodes in the phylogenetic tree. The ancestral nodes were sorted by their estimated median node age and each unit in the x axis indicated different ancestral nodes. The error bar represents the 95% highest posterior density (HPD) interval. 12-20 founder strains are projected based on nodes having the lower endpoint of the 95% highest posterior probability density older than the host. The labels on the y axis were rescaled for confidentiality. (FIG. 3C) Negative association between transmission and the diversification of subpopulations. Each data point shows the expected transmission rate (estimated by BEAST) and the FST value between a pair of skin sites. * indicates linear regression lines with significant slopes (p<0.05). The highlighted data points indicate either the umbilicus or toeweb in a pairwise comparison. (FIG. 3D) Consistency of Bayes factors (upper) and posterior probabilities (lower) supporting pairwise transmission events estimated from two independent MCMC runs.

FIGS. 4A-4E. Gene content diversity of the subject isolates. (FIG. 4A) Gene accumulation curves for the subject-specific pan-genomes (5476-6436 gene clusters) and core-genomes (954-1325 gene clusters), or that of the 50 public isolates, as a function of the number of sequenced isolates. Error bars show the standard deviation for 10 simulations. (FIG. 4B) Shared vs. unique subject-specific pan- and core-genes in the subject isolates and public strains. (FIG. 4C) Diversity of the subject isolates based on presence and absence of accessory genes. Leaf nodes are shaded by the skin site of origin; the background shading indicates the subject. A cluster containing toeweb isolates from all five subjects is highlighted. (FIG. 4D) Distribution of S. epidermidis genes in p0 with respect to their variability across skin sites (see FIG. 5D for other subjects). An example cluster of genes with high variability is highlighted with a box (boundaries arbitrarily selected), and their prevalence shown in the heatmap. Each row in the heatmap represents a unique S. epidermidis gene, and the row and column hierarchical clusters were generated based on Euclidean distances. (FIG. 4E) The COG functional categories of representative toeweb genes (i.e. present in >40% of the toeweb isolates but <10% in any of the other skin sites, n=28).

FIGS. 5A-5J. Spatio-temporal distribution of S. epidermidis functional features. (FIG. 5A) Shared vs. unique S. epidermidis gene clusters at different time points. Related to FIG. 4. The total number of time points at which at least one isolate was successfully cultured from the subpopulation is shown on the top of each graph. Subpopulations with significant temporal changes are marked with an “*”. The right index in p0 was marked with a triangle because a limited number of isolates (n=2) were cultured from one of the time points. (FIG. 5B) The relationship between sample sizes and p-values of temporal changes. For each given subpopulation (data point), the time point at which the lowest number of isolates were cultured, the highest number of isolates were cultured, and the total number of isolates cultured across time points were visualized. (FIG. 5C) Comparison of p-values of temporal changes including or excluding rare genes. Permutation analyses were run separately with or without filtering out rare genes (defined as those S. epidermidis genes that were present in only one isolate in that subpopulation). Benjamini-Hochberg adjusted p-values of the analyses were then compared to validate that the statistical significance were robust to the presence of rare genes. (FIG. 5D) The distribution of S. epidermidis genes with respect to their variability across skin sites (see FIG. 4D for subject p0). Example clusters of genes with high variability are highlighted with boxes (boundaries arbitrarily selected). Skin-site distribution of the genes in each of the highlighted clusters and their prevalence were shown in the heatmaps. Each row in the heatmap represents a unique S. epidermidis gene, and the row and column hierarchical clusters were generated based on Euclidean distances. (FIG. 5E) Examples of KEGG modules with differential representation across subjects and skin sites (for a full list, see Table 4). Module representation (the proportion of KEGG orthologs in the module present in an isolate) was rescaled proportionally by the mean module representation at each skin site. (FIG. 5F) Prevalence of predicted BGCs across subjects and skin sites. (FIG. 5G) SNP-based gene tree of the nrps and siderophore BGCs and their distribution across subjects and skin sites. The skin site of each BGC-carrying isolate is indicated in green. (FIG. 5H) SNP-based gene tree of the terpene BGCs and their distribution across subjects and skin sites. The skin site of each BGC-carrying isolate is indicated in green. (FIGS. 5I-5J) distribution of different types of bacteriocin (FIG. 5I) and lantipeptide (FIG. 5J) BGCs across subjects and skin sites. Each “type” represents BGC sequences clustered at 80% sequence identity. No gene tree was constructed for these BGCs due to the lack of colinear regions.

FIG. 6. Contig read coverages as a function of contig sizes. The plot contained 50000 randomly sampled data points.

FIGS. 7A-7D. Diversification of sister isolates driven by potential HGT events. (FIG. 7A) Gene content heterogeneity—the proportion of genes that are only found in one isolate of a pair of isolates—as a function of pairwise core-genome nucleotide differences. For visualization, the plot includes only 10000 randomly sampled data points. Gene content heterogeneity between sister isolates are highlighted with a box. (FIG. 7B) Functional annotation of the differential genes. All differential genes were mapped to KEGG orthologs (the annotations of the KEGG orthologs were shown when available) and their prevalence within sister isolate groups is shown. The p-value shows the probability of observing the differential prevalence solely due to genome incompleteness. The error bars show the standard deviation across sister isolate groups. (FIG. 7C) Presence of differential genes in the 20 unique mobile-element-like contigs identified using PlasFlow. The heatmap shows the fraction of nucleotides in the mobile-element-like contigs that was aligned to the 25 chromosome-like contigs identified containing differential genes. The error bars show the standard deviation across sister isolate groups. Two predicted phage sequences (nearly 100% alignment over contig length) are indicated by arrows. (FIG. 7D) gene content of the predicted phage sequences indicated in (FIG. 7C). Note that the sequences are visualized in a circular layout but are not necessarily circular DNAs.

FIG. 8 Spatio-temporal distribution of sister isolates. Each panel shows the number of sister isolates found at different time points (upper) and the total number of skin sites that contained at least one sister isolate from that group (lower).

FIGS. 9A-9D. ABR genes encoded by predicted S. epidermidis plasmids. (FIG. 9A) Prevalence of predicted plasmid segments (i.e., the proportion of isolates carrying the predicted plasmid segments) across subjects and skin sites. The row and column hierarchical clusters were generated based on Euclidean distances. This panel is related to FIG. 10B, which uses a different plasmid prediction method. (FIG. 9B) Prevalence of predicted plasmid-encoded ABR. The heatmap shows the number of predicted plasmid segments that conferred resistance to both the row and the column antibiotics. (FIG. 9C) Host-specific distribution of predicted MDR plasmid segments. The ABR genes (and the respective antibiotics they confer resistance to) encoded by two predicted MDR plasmid segments are shown. Note that sequences are visualized in a circular layout but were not gap-closed. (FIG. 9D) MIC50 and MIC90 of selected antibiotics and their association with predicted plasmid-encoded ABR genes. Two isolates (0995 and 1085) that conferred resistance to all six tested antibiotics were indicated by arrows.

FIGS. 10A-10D. Distribution of predicted plasmid-encoded ABR genes. (FIG. 10A) Prevalence of predicted phage sequences (i.e., the proportion of isolates carrying the predicted phage sequences) across subjects and skin sites. The row dendrogram shows the diversity of the predicted phages based on the presence and absence of gene contents and is shaded based on the closest phage reference sequence as predicted by PHASTER. The column hierarchical clusters were generated based on Euclidean distances. (FIG. 10B) Prevalence of predicted plasmid contigs (that aligned to PLSDB) across subjects and skin sites. The row and column hierarchical clusters were generated based on Euclidean distances. This panel is related to FIG. 9A. (FIG. 10C) Skin-site prevalence of predicted plasmid-encoded ABR genes that were only observed in a single subject. Prevalence was defined as the proportion of isolates in a subpopulation that carried at least one predicted plasmid segment which encoded resistance to the antibiotic in question. (FIG. 10D) Skin-site prevalence of predicted plasmid-encoded ABR that were observed in at least two subjects. The subjects with no predicted plasmid-encoded resistance to a given antibiotic were shown with increased transparency. Prevalence was defined as the proportion of isolates in a subpopulation that carried at least one predicted plasmid segment which encoded resistance to the antibiotic in question.

FIGS. 11A-11C. Predicted S. epidermidis genes and variants that can affect virulence. (FIG. 11A) Prevalence of known S. epidermidis virulence genes across subjects and skin sites. (FIG. 11B) Mutations that split the transmembrane domains of AgrC, as verified with Sanger sequencing. (FIG. 11C) Genes involved in the TCA cycle pathway, as an example of carbohydrate metabolism genes that showed higher expression levels with the presence of population supernatant in the agr interference experiments.

FIGS. 12A-12F. Variability at the agr locus and variation in predicted virulence expression. (FIG. 12A) Novel sequence types of the agrABCD operon and prevalence across the relevant subjects and skin sites. Amino acid sequences of the two novel agrD genes, verified with Pacbio sequencing, are shown. (FIG. 12B) Quorum sensing interference of agr Type I-III isolates by Type IV supernatant. ddCt values were obtained by subtracting dCT values measured at zero hours from dCT values measured at four hours. *: Welch's t-test on ddCt values p<0.05. **: Welch's t-test on ddCt values p<0.01. At least 3 biological replicates were performed for each experiment. (FIG. 12C) Quorum sensing interference of an agr Type IV isolate by Type I-III supernatant, as in FIG. 12B. (FIG. 12D) Distribution and dominance type frequency of the three canonical agr types (Type I-III) across subjects and skin sites. (FIG. 12E) Quorum sensing interference of agr Type I-III isolates by population supernatant generated by mixed cultures, as in FIG. 12B. (FIG. 12F) S. epidermidis operons showing significantly lower expression levels with the presence of population supernatant.*: Welch's t-test on ddCt values p<0.05. **: Welch's t-test on ddCt values p<0.01.

FIGS. 13A-13H. Association between S. epidermidis gene prevalence and the local skin microbiota. (FIGS. 13A-13D) Taxonomic and gene content compositions of the skin microbiota. Principal component analyses of the microbiome taxonomic compositions on species level were conducted to illustrate the diversification of skin microbiome compositions across subjects (FIG. 13A) and skin sites (FIG. 13B). The five loading vectors with the largest norms are visualized on the plot. Similarly, principal component analyses of the microbiome gene coverage were conducted to illustrate the diversification of coding potentials of the skin microbiota across subjects (FIG. 13C) and skin sites (FIG. 13D). (FIG. 13E) A diagram outlining the training and evaluation of the recursive partitioning tree model. (FIGS. 13F-H), Given the variability of the S. epidermidis genes across subpopulations (i.e. Pielou's index of gene prevalence levels, x axis), the prior predictability of the S. epidermidis gene prevalence in the new host (i.e. test set) (FIG. 13F), and the increased predictability when including skin site specification (FIG. 13G) and contextual microbiome features (FIG. 13H) are shown. The top 20 genes that had the greatest increase in predictability when including skin site specification or microbiome features were highlighted.

FIGS. 14A-14B. Association of S. epidermidis gene prevalence with contextual environment. (FIG. 14A) S. epidermidis genes whose prevalence were significantly associated with at least one of the principal components that described microbiome composition. (FIG. 14B) Function and plasmid association of the microbiome-dependent S. epidermidis genes. The COG functional categories (upper) of the top 20 genes that had the greatest increase in predictability when including skin site specification or microbiome features are shown, as well as their presence in predicted (via PlasFlow) and known (via PLSDB) plasmids (lower).

### DETAILED DESCRIPTION

Provided herein is an in-depth survey of within-individual, population-level diversity in the human skin microbiome. It was previously hypothesized, using limited metagenomic inferences, that phylogenetically diverse strains could coexist in the skin (Oh et al., 2014). In an extensive genomic and metagenomic survey of 1482 isolates cultivated from healthy skin, it has been conclusively demonstrated that each subject was colonized by diverse S. epidermidis strains from both dominant phylogenetic clades identified in the initial assessments of S. epidermidis phylogenetic variation (Conlan et al., 2012). Herein, host-specificity, skin site specialization, and evolutionary and demographic events were deeply probed to provide new insights into the spatio-temporal diversity and function of a commensal skin bacteria.

### Staphylococcus Epidermidis

The present disclosure provides, in some aspects, an admixture of Staphylococcus epidermidis (S. epidermidis) cells comprising at least two S. epidermidis cell types selected from S. epidermidis Type I cells, S. epidermidis Type II cells, S. epidermidis Type III cells, S. epidermidis Type IIIb cells, and S. epidermidis Type IV cells.

S. epidermidis is a Gram-positive, coccoid-shaped, commensal bacteria that is part of normal mammalian skin and mucous microbiomes. It is distinguished from the pathogenic Staphylococcus aureus by being coagulase-negative. S. epidermidis predominantly colonizes the axillae, elbow, knee, hands, feet, head, and umbilicus, although the colonization pattern varies from subject to subject.

Although not generally dangerous, S. epidermidis is an opportunistic pathogen in immunocompromised hosts. In particular, S. epidermidis is the most frequent causative bacteria in infections of indwelling medical devices, such as catheters, prosthetic joints, vascular grafts, central nervous system shunts, and heart valves (see, e.g., Otto, “Staphylococcus epidermidis—the “accidental pathogen”, Nat. Rev. Microbiol., 2010, 7(8): 555-567). Among other bacteria that cause infections, S. epidermidis is particularly difficult for the innate immune system to clear due to its ability to form and exist in three-dimensional biofilms that are resistant to antibiotics (e.g., penicillins, aminoglycosides, and quinolones) and lymphocyte phagocytosis.

S. epidermidis Types

S. epidermidis regulates gene expression in response to fluctuations in bacterial population density by quorum sensing. Quorum sensing regulates bacteria (e.g., S. epidermidis) phenotype expression, which in turn modulates bacterial behaviors. Quorum sensing bacteria (e.g., S. epidermidis) produce and release chemical signal molecules called autoinducers that increase in concentration as a function of cell density. Once a minimal concentration threshold for autoinducers is reached, changes in gene expression occur in the bacteria (e.g., S. epidermidis). S. epidermidis use quorum sensing to regulate cellular processes including, but not limited to, virulence, symbiosis conjugation, and biofilm formation.

S. epidermidis virulence is regulated by quorum sensing using the accessory gene regulatory (agr system). Sequencing studies have revealed three different classes of S. epidermidis agr systems in clinical isolates (DUfour P. et al. J. Bacteriol. 2002; 184:1180-1186), which are referred to here as agr Types I, II, and III. See also Olsen M E et al. J Bacteriol. 2014; 196(19): 3482-93. The data provided herein identified two additional agr Types: Types IIIb and IV. The agr system is controlled by an extracellular peptide signal known as an autoinducing peptide (AIP), which is secreted during growth and activates gene expression in a cell-density dependent manner. The agr locus in S. epidermidis is composed of the agrABCD operon, which encodes the core machinery for producing and detecting AIPs. AgrD is the propeptipe precursor that is secreted and processed by AgrB, an integral membrane peptidase that pairs with a signal peptidase to secrete AIP extracellularly. Accumulation of AIP is sensed by the histidine kinase AgrC, which initiates a signal cascade resulting in ArgC phosphorylating the regulator AgrA. Phosphorylated AgrA results in the induction of expression from the agr P2 and P3 promoters, which drive expression of the transcription factor RNAIII.

S. epidermidis types are categorized based on the AgrD sequence in the agrABCD operon. There are more S. epidermidis strains than there are S. epidermidis types, so an admixture of S. epidermidis cells may contain, for example, at least 5 (e.g., 5, 6, 7, 8, 9, 10 or more) S. epidermidis strains while simultaneously containing only 2 S. epidermidis types. A summary of the AgrD sequences in Types I, II, III, IIIb, and IV is shown below in Table 1.

TABLE 1
AgrD Type Sequences

| | | AIP-containing | | SEQ |
|---|---|---|---|---|
| Type | Leader | region | Tail | ID NO: |
| I* | MENIFNLFIK | TVAGDSVCASYF | DEPEVPE | 1 |
| | FFTTILEFIG | | ELTKLYE | |
| II* | MNLLGGLLLK | NASKYNPCSNYL | DEPQVPE | 2 |
| | IFSNFMAVIG | | ELTKLDE | |
| III* | MNLLGGLLLK | NAAKYNPCASYL | DEPQVPE | 3 |
| | LFSNFMAVIG | | ELTKLDE | |
| IIIb⁺ | MNLLGGLFLK | NAAKYNPCASYL | DEPQVPE | 4 |
| | IFSNFMAVIG | | ELTKLDE | |
| IV⁺ | MNLLGGLLLK | NASKYNPCVMYL | DEPQVPE | 5 |
| | IFSNFMAVIG | | ELTKLDE | |

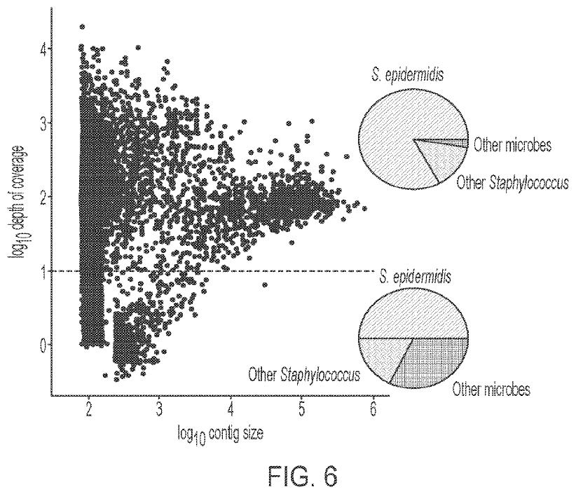

*see Dufour, et al., “High genetic variability of the agr locus in Staphylococcus species,” J. Bacteriol, 2002, 184: 1180-1186.
⁺see Example 4 and Figure 6

In some embodiments, an admixture contains 2-500 S. epidermidis types. In some embodiments, an admixture contains 5-100, 50-250, 10-200, 25-400, 75-350, or 100-500 S. epidermidis types. In some embodiments, an admixture contains 2, 5, 10, 25, 50, 75, 100, 125, 150, 175, 200, 225, 250, 275, 300, 325, 350, 375, 400, 425, 450, 475, or 500 S. epidermidis types.

In some embodiments, an admixture contains genetically engineered S. epidermidis cell types (see, e.g., US 2019/0040116, published Feb. 7, 2019). Genetically engineered refers to genome modifications to naturally-occurring strains (e.g., S. epidermidis strains). Genome modifications may be introduced by any method known in the art including, but not limited to: clustered regularly interspaced short palindromic repeats (CRISPR/Cas) (see, e.g., Cong, et al., “Multiplex genome engineering using CRISPR/Cas systems,” Science, 2013, 339(6121): 819-823), zinc finger nucleases (see, e.g., Proteus and Baltimore, “Chimeric nucleases stimulate gene targeting in human cells,” Science, 2003, 300(5620): 763), and transcription activator-like effectors (see, e.g., Boch, et al., “Breaking the code of DNA binding specificity in TAL-type III effectors,” Science, 2009: 326(5959): 1509-1512). In some embodiments, the genetically engineered S. epidermidis types have a biological advantage over non-genetically engineered (e.g., naturally occurring) types. The biological advantage may be any advantage known in the art, including, but not limited to: colonization of additional sites, increased survival, increased proliferation, antibiotic resistance, virulence, and/or biofilm formation.

In some embodiments, an admixture contains 2-500 genetically engineered S. epidermidis types. In some embodiments, an admixture contains 5-100, 50-250, 10-200, 25-400, 75-350, or 100-500 genetically engineered S. epidermidis types. In some embodiments, an admixture contains 2, 5, 10, 25, 50, 75, 100, 125, 150, 175, 200, 225, 250, 275, 300, 325, 350, 375, 400, 425, 450, 475, or 500 genetically engineered S. epidermidis types.

Admixtures of S. epidermidis

As indicated above, there are currently five S. epidermidis types known. Other S. epidermidis types that are yet to be identified may also be include in admixtures of the present disclosure. An admixture of S. epidermidis cells is a mixture that comprises at least two S. epidermidis types. In some embodiments, an admixture comprises three or more, four or more, or five S. epidermidis types. The skilled person will understand that an admixture of S. epidermis cells may contain any combination of Type I, Type II, Type III, Type IIIb, and Type IV S. epidermis cells.

In some embodiments, an admixture comprises Types I, II, and III S. epidermidis cells. In some embodiments, an admixture comprises Types I, II, III, IIIb, and IV S. epidermidis cells. In some embodiments, an admixture comprises Types I, II, III, and IIIb S. epidermidis cells. In some embodiments, an admixture comprises Types I and II, Types I and III, Types I and IIIb, or Types I and IV S. epidermidis cells. In some embodiments, an admixture comprises Types I, II, and IIIb; Types I, II, and IV, Types II, III, and IIIb, or Types II, III, and IV S. epidermidis cells. In some embodiments, an admixture comprises Types III, IIIb, and IV S. epidermidis cells. In some embodiments, an admixture comprises Types III, IIIb, and IV S. epidermidis cells. In some embodiments, an admixture comprises Types II and III, Types II and IIIb, or Types II and IV S. epidermidis cells. In some embodiments, an admixture comprises Types III and IIIb or Types III and IV S. epidermidis cells. In some embodiments, an admixture comprises Types IIIb and IV S. epidermidis cells.

An admixture comprising at least two S. epidermidis types may comprise an unequal distribution or an equal distribution of S. epidermidis cell Types. In an unequally distributed admixture, the percentages of cell Types in the admixture are not equal. In some embodiments, an unequally distributed admixture of two S. epidermidis cell Types comprises one type (e.g., Type I, Type II, Type III, Type IIIb, or Type IV) at a higher percentage than the other cell Type. In some embodiments, an unequally distributed admixture of three S. epidermidis cell Types comprises one type (e.g., Type I, Type II, Type III, Type IIIb, or Type IV) at a higher or lower percentage than the other two cell Types, and so on. In an equally distributed admixture, the percentages of the admixture of each of the S. epidermidis cell Type are not equal. In some embodiments, an unequally distributed admixture of two S. epidermidis cell Types comprises one type (e.g., Type I, Type II, Type III, Type IIIb, or Type IV) at a higher percentage than the other cell Type. In some embodiments, an unequally distributed admixture of three S. epidermidis cell Types comprises one type (e.g., Type I, Type II, Type III, Type IIIb, or Type IV) at a higher or lower percentage than the other two cell Types, and so on. In an unequally distributed admixture, the percentages of cell Types in the admixture are equal. In some embodiments, an equally distributed admixture of two S. epidermidis cell Types comprises one cell Type (e.g., Type I, Type II, Type III, Type IIIb, or Type IV) at a percentage equal to the other cell Type.

### Gene Expression

In some aspects of the present disclosure, contacting skin cells (e.g., S. epidermidis that has colonized skin cells) of a subject with an admixture of S. epidermidis cells in which there are at least two types of S. epidermidis present modifies gene expression compared to control cells. Control cells may be S. epidermidis cells that colonize the skin cells of the same subject that are not contacted with an admixture of S. epidermidis cells and/or S. epidermidis cells that colonize the skin cells of another subject that are not contacted with an admixture of S. epidermidis cells. Modified expression may be either decreased gene expression or increased gene expression. The statistical significance of modified expression levels may be determined by any method known in the art including, but not limited to, Welch's t-test, Pearson's t-test, Student's t-test, and one-way analysis of variance (ANOVA). Gene expression may be measured by any method known in the art including, but not limited to, quantitative reverse transcription PCR (qRT-PCR), microarray analysis, Northern blot, serial analysis of gene expression (SAGE), Western blot, and RNA Seq.

Any gene expressed in S. epidermidis cells that colonize skin cells may have its expression modified by being contacted with an admixture of S. epidermidis cells. In some embodiments, contacting skin cells of a subject with an admixture of S. epidermidis cells in which there are at least two types of S. epidermidis present modifies gene expression of nitrogen respiration genes, urease activity genes, carbohydrate metabolism genes, iron uptake genes, sulfur metabolism genes, and/or virulence factor genes.

### Nitrogen Respiration Genes

In some embodiments, the expression of nitrogen respiration genes in S. epidermidis cells that colonize skin cells is modified by an admixture of S. epidermidis cells. Nitrogen respiration in S. epidermidis occurs in two steps: (i) nitrate uptake and reduction by a dissimilatory nitrate reductase to nitrite, which is subsequently excreted; and (ii) after depletion of nitrate, the accumulated nitrite is imported and reduce by an NADH-dependent nitrite reductase to ammonia. Proteins involved in nitrogen respiration in S. epidermidis include, but are not limited to: nitrate transporter protein (narT), which transports nitrate into S. epidermidis cells; cytoplasmic histidine kinase (nreB), which phosphorylates targets under oxygen-depleted conditions; nitrate/nitrite sensor protein X (narX), which transduces signals of nitrate availability to the narL protein and of both nitrate and nitrite to the narP protein; nitrate reductase protein (narH, narG, nasD and nasE), which transfers electrons to and reduces nitrate; and S-adenosyl-L-methionine uroporphirynogen III C-methyltransferase (sumT), which participates in cysteine biosynthesis.

Any gene involved in nitrogen respiration in S. epidermidis may have its expression modified by being contacted with an admixture of S. epidermidis cells. In some embodiments, the nitrogen respiration gene whose expression is modified is selected from narT, nreC, nreB, narX, narH, narG, sumT, nasE, and nasD.

Urease Activity Genes

In some embodiments, the expression of urease activity genes in S. epidermidis cells that colonize skin cells is modified by an admixture of S. epidermidis cells. Urease catalyzes the hydrolysis of urea into two molecules of ammonia and one molecule of carbon dioxide in aqueous environments. In general, urease activity protects bacteria (e.g., S. epidermidis) in acidic environments by neutralizing acids. Thus, treating bacterial (e.g. S. epidermidis) cultures with acids or acidifying the environment in which bacterial cultures exist upregulates urease activity. S. epidermis urease is a multimeric enzyme composed of three structural subunits: UreA, UreB, and UreC. Urease is activated by the accessory proteins UreD, UreE, UreF, UreG, and UreH.

Any gene involved in urease activity in S. epidermidis may have its expression modified by being contacted with an admixture of S. epidermidis cells. In some embodiments, the urease activity gene whose expression is modified is selected from ureA (Gene ID: 1057998), ureB (Gene ID: 1055719), ureC (Gene ID: 1057996), ureD (Gene ID: 1055730), ureE (Gene ID: 1055725), and ureF (Gene ID: 1057994).

Carbohydrate Metabolism Genes

In some embodiments, the expression of carbohydrate metabolism genes in S. epidermidis cells that colonize skin cells is modified by an admixture of S. epidermidis cells. S. epidermidis can use glucose, sucrose, and lactose to form acid products during carbohydrate metabolism. Carbohydrate metabolism genes may participate in any carbohydrate metabolism pathway in S. epidermidis including, but not limited to: glycolysis, the citric acid cycle,

In some embodiments, the expression of carbohydrate metabolism genes in S. epidermidis cells whose expression is modified participate in glycolysis. Glycolysis is the enzymatic pathway to breaks down glucose into 2 molecules of pyruvate and produces ATP and NADH. Non-limiting examples of proteins that participate in glycolysis include: fructose 1-phosphate kinase (FruB), glucosamine 6-phosphate synthetase (GlmS), fructose bisphosphate aldolase (Fba), transketolase (Tkt), triose-phosphate isomerase (Tpi), glyceraldehyde 3-phosphate dehydrogenase (Gap, GapB), phosphoglycerate isomerase (Pgi), phosphoglycerate kinase (Pgk), enolase (Eno), and phophosenolpyruvate carboxykinase (PEPCK).

In some embodiments, the expression of carbohydrate metabolism genes in S. epidermidis cells whose expression is modified participate in the citric acid cycle (also known as the tricarboxylic acid cycle). The citric acid cycle is the enzymatic breakdown of acetyl-coA into oxaloacetate and produces NADH, GTP, and FADH₂. Non-limiting examples of proteins that participate in the citric acid cycle include: aconitase hydratase (CitB), isocitrate dehydrogenase (CitC), alpha-ketoglutarate dehydrogenase (OgdH), succinyl-CoA synthetase (Sucla2), succinate dehydrogenase (SdhA), fumarate hydratase (CitG), malate dehydrogenase (MDH1), and phosphoenolpyruvate carboxykinase (PckA).

In some embodiments, the expression of carbohydrate metabolism genes in S. epidermidis cells whose expression is modified participate in fermentation. Fermentation is the extraction of energy (e.g., ATP) from carbohydrates in the absence of oxygen. Non-limiting examples of proteins that participate in fermentation include lactate dehydrogenase (LDH), pyruvate dehydrogenase (PDH), short-chain acyl-CoA dehydrogenase (ScaD), alcohol dehydrogenase (PflB), formate dehydrogenase (Fdh), and acetoin reductase (BdhA).

Any gene involved in carbohydrate metabolism in S. epidermidis may have its expression modified by being contacted with an admixture of S. epidermidis cells. In some embodiments, the carbohydrate metabolism gene whose expression is modified is selected from FruB, GlmS, Fba, Tkt, Tpi, Gap, GapB, Pgi, Pgk, Eno, PepcK, CitB, CitC, OgdH, Sucla2, SdhA, CitG, Mdh1, PckA, LDH, PDH, ScaD, PflB, Fdh, and BdhA.

Iron Uptake Genes

In some embodiments, the expression of iron uptake genes in S. epidermidis cells present on skin cells is modified by an admixture of S. epidermidis cells. Commensal and pathogenic bacteria (e.g., S. epidermidis) require iron to proliferate, and are adapted to remove iron from the host proteins transferrin and lactoferrin. S. epidermidis bacteria contain the transferrin-binding protein glyceraldehyde-3-phosphate dehydrogenase in its cell walls that binds the protein transferrin. This binding removes the iron from transferrin, which is transferred to surface lipoproteins and transport proteins in the S. epidermidis cell wall before being transferred into the cell. Proteins involved in iron uptake in S. epidermidis include, but are not limited to, iron (III) dicitrate transport system permease protein (fecD), which helps to transport iron across the membrane; iron-uptake system permease protein (feuC), an ATP-dependent membrane transporter; iron (III) dicitrate transport ATP-binding protein (FecE), which couples energy to the iron transport system; and petrobactin-binding protein (YclQ), which selectively binds iron-free and ferric petrobactin.

Any gene involved in iron uptake in S. epidermidis may have its expression modified by being contacted with an admixture of S. epidermidis cells. In some embodiments, the iron uptake gene whose expression is modified is selected from fecD, feuC, fecE, and yclQ.

Sulfur Metabolism Genes

In some embodiments, the expression of sulfur metabolism in S. epidermidis cells present on skin cells is modified by an admixture of S. epidermidis cells. Sulfur metabolism refers to the oxidation or reduction of sulfur. Sulfate-oxidizing bacteria (e.g., S. epidermidis) produce ATP as reduced sulfur forms (e.g., sulfite, hydrogen sulfide, elemental sulfur, thiolsulfate) are oxidized to sulfur. Sulfate-reducing bacteria (e.g., S. epidermidis) reduce sulfate and other oxidized sulfur compounds, such as sulfite, thiosulfate, and elemental sulfur, to sulfide. The purpose of reducing the sulfate is to produce energy using the enzymes ATP sulfurylase, APS reductase, and sulfite reductase, and sulfide is excreted. Proteins involved in sulfur metabolism include, but are not limited to: phosphoadenosine phosphosulfate reductase (cysH), which reduces activated sulfate into sulfite; sulfite reductase subunit alpha (cysJ) and sulfite reductase subunit beta (cysI), components of the sulfite reductase complex that catalyzes the 6-electron reduction of sulfite to sulfide; precorrin-2 dehydrogenase (sirC), a transmembrane iron transporter; sulfate adenylyltransferase (sat), which catalyzes the formation of adenosine 5′-phosphosulfate from ATP and sulfate; adenylyl sulfate kinase (cysC), which catalyzes the synthesis of activated sulfate; and cysteine tRNA ligase (cysS), which catalyzes the formation of L-cysteinyl-tRNA^{cys}.

Any gene involved in sulfur metabolism in S. epidermidis may have its expression modified by being contacted with an admixture of S. epidermidis cells. In some embodiments, the iron uptake gene whose expression is modified is selected from cysH, cysJ, cysI, sirC, sat, cysC, and cysS (Gene ID: 1058061).

Virulence Factor Genes

In some embodiments, the expression of virulence factor genes in S. epidermidis cells present on skin cells is modified by an admixture of S. epidermidis cells. Virulence factors are proteins produced by bacteria (e.g., S. epidermidis) that increase their survival, proliferation, and immunoevasion. Proteins that are virulence factors in S. epidermidis include, but are not limited to: intracellular adhesion protein A (icaA), intracellular adhesion protein B (icaB), intracellular adhesion protein C (icaC), intracellular adhesion protein D (icaD), and intracellular adhesion protein R (icaR), which produce the expopolysaccharide polysaccharide intercellular adhesion (PIA) that is important for biofilm formation; autolysin (atl) and autolysin E (altE), which excrete cytoplasmic proteins and acts as an adhesion molecule; extracellular matrix binding protein (ebh), extracellular matrix binding protein A (ebhA), and extracellular matrix binding protein B (ebhB), which promote attachment to both soluble and immobilized forms of fibronectin; elastin binding protein (ebp), which binds elastin; glycerol ester hydrolase (geh) and glycerol ester hydrolase D (gehD), which may promote survival in abscesses; beta hemolysin (hlb), which is a phospholipase C enzyme with specific activity towards sphingomyelins; triacylglycerol lipase (lip), which hydrolyzes fatty alcohols; nuclease (nuc), which digests single-stranded nucleic acids; phenol-soluble modulin beta (psmB), which acts as a toxin and assists in biofilm formation and colony spreading; serine-aspartate repeat protein F (sdrF), which mediates binds type I collagen in biofilm formation; serine-aspartate repeat protein H (sdrH), which is expressed on the surface of S. epidermidis cells; autolysin (se2319), which is a lysin protein expressed on the surface of S. epidermidis cells; glutamyl endopeptidase (sspA), which is the most important protease for degradation of fibronectin-binding protein (FnBP) and surface protein A; and staphopain B (sspB), which is a cysteine protease that degrades host elastin, fibrogen, fibronectin, and kininogen.

Any gene that encodes a virulence factor in S. epidermidis may have its expression modified by being contacted with an admixture of S. epidermidis cells. In some embodiments, the virulence factor gene whose expression is modified is selected from icaA, icaB, icaC, icaD, icaR, ad, atlE, ebh, ebhA, ebp, geh, gehD, hlb, lip, nuc, psmB, sdrF, sdrH, se2319, sspA, and sspB.

Levels of Expression Changes

Modified gene expression in S. epidermidis cells on skin contacted with an admixture of S. epidermidis cells may be decreased gene expression or increased gene expression. In some embodiments, gene expression is decreased compared to a control. In some embodiments, gene expression is increased compared to a control. In some embodiments, gene expression of some genes is decreased and gene expression of some genes is increased.

In some embodiments, gene expression is decreased by between 1.5-fold and 12-fold. In some embodiments, gene expression is decreased by between 1.0-fold and 10-fold, 2.0-fold and 5.0-fold, 4.0-fold and 12-fold, or 3-fold and 9-fold. In some embodiments, gene expression is decreased by 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, or 12.0-fold.

In some embodiments, gene expression is decreased by between 10% and 100%. In some embodiments, gene expression is decreased by between 20% and 90%, 30% and 80%, 40% and 70%, or 50% and 60%. In some embodiments, gene expression is decreased by 10%, 15%, 20%, 25%, 30%, 35%, 40%, 45%, 50%, 55%, 60%, 65%, 70%, 75%, 80%, 85%, 90% or 100%.

In some embodiments, gene expression is increased by between 1.5-fold and 12-fold. In some embodiments, gene expression is increased by between 1.0-fold and 10-fold, 2.0-fold and 5.0-fold, 4.0-fold and 12-fold, or 3-fold and 9-fold. In some embodiments, gene expression is increased by 1.5-fold, 2.0-fold, 2.5-fold, 3.0-fold, 3.5-fold, 4.0-fold, 4.5-fold, 5.0-fold, 5.5-fold, 6.0-fold, 6.5-fold, 7.0-fold, 7.5-fold, 8.0-fold, 8.5-fold, 9.0-fold, 9.5-fold, 10.0-fold, 10.5-fold, 11.0-fold, 11.5-fold, or 12.0-fold.

In some embodiments, gene expression is increased by between 10% and 100%. In some embodiments, gene expression increased by between 20% and 90%, 30% and 80%, 40% and 70%, or 50% and 60%. In some embodiments, gene expression is increased by 10%, 15%, 20%, 25%, 30%, 35%, 40%, 45%, 50%, 55%, 60%, 65%, 70%, 75%, 80%, 85%, 90% or 100%.

### Compositions

In some embodiments of the present disclosure, an admixture is present in a composition. A composition comprising an admixture, for example, may comprise any amount of the admixture that is a therapeutically effective amount. In some embodiments, a composition comprises at least 0.01%, 0.05%, 0.1%, 0.2%, 0.3%, 0.4%, 0.5%, 0.6%, 0.7%, 0.8%, 0.9%, 1.0%, 1.5%, 2.0%, 3.0%, 4.0%, 5.0%, 6.0%, 7.0%, 8.0%, 9.0%, 10.0%, 11.0%, 12.0%, 13.0%, 14.0%, 15.0%, 16.0%, 17.0%, 18.0%, 19.0%, 20.0%, 25.0%, 30.0%, 35.0%, 40.0%, 45.0%, 50.0% or more by weight of the admixture of S. epidermidis cells. In some embodiments, the upper limit is 90.0% by weight of the admixture of S. epidermidis cells.

In some embodiments, a composition comprises 0.01% to 30%, 0.01% to 20%, 0.01% to 5%, 0.1% to 30%, 0.1% to 20%, 0.1% to 15%, 0.1% to 10%, 0.1% to 5%, 0.2% to 5%, 0.3% to 5%, 0.4% to 5%, 0.5% to 5%, 1% to 5%, or more by weight of the of the admixture of S. epidermidis cells.

In some embodiments, a composition contains 2-500 S. epidermidis types. In some embodiments, a composition contains 5-100, 50-250, 10-200, 25-400, 75-350, or 100-500 S. epidermidis types. In some embodiments, a composition contains 2, 5, 10, 25, 50, 75, 100, 125, 150, 175, 200, 225, 250, 275, 300, 325, 350, 375, 400, 425, 450, 475, or 500 S. epidermidis types.

In some embodiments, a composition is a topical composition. A topical composition is formulated for topical administration to a subject in need thereof. In some embodiments, a topical composition contains a pharmaceutical excipient.

Dosage forms for the topical or transdermal administration include powders, sprays, ointments, pastes, creams, lotions, gels, solutions, patches, drops and inhalants. An admixture of S. epidermidis cells may be mixed under sterile conditions with a suitable pharmaceutically-acceptable excipient (e.g., diluent and/or carrier). Thus, ointments, pastes, creams and gels may contain excipients. Powders and sprays may contain excipients and/or propellants.

In some embodiments, a formulation is a topical formulation. A topical formulation may be, for example, in any form suitable for application to the body surface, such as a cream, lotion, sprays, solution, gel, ointment, paste, plaster, paint, bioadhesive, suspensions, emulsions, or the like, and/or can be prepared so as to contain liposomes, micelles, and/or microspheres. Such a formulation can be used in combination with an occlusive overlayer so that moisture evaporating from the body surface is maintained within the formulation upon application to the body surface and thereafter. In some embodiments, a formulation can include a living cell culture composition and can comprise an admixture of S. epidermidis cells. This living cell culture composition of the admixture of S. epidermidis cells may be delivered, for example, directly to the skin for treating or preventing abnormal skin conditions associated with S. epidermidis.

Topical formulations include those in which any other active ingredient(s) is (are) dissolved or dispersed in a dermatological vehicle known in the art (e.g., aqueous or nonaqueous gels, ointments, water-in-oil or oil-in-water emulsions). Constituents of such vehicles can comprise water, aqueous buffer solutions, non-aqueous solvents (such as ethanol, isopropanol, benzyl alcohol, 2-(2-ethoxyethoxy)ethanol, propylene glycol, propylene glycol monolaurate, glycofurol or glycerol), oils (e.g., a mineral oil such as a liquid paraffin, natural or synthetic triglycerides such as MIGLYOL™, or silicone oils, such as dimethicone). Depending, inter alia, upon the nature of the formulation as well as its intended use and site of application, the formulation used can contain at least one component (for example, when the formulation is an aqueous gel, components in addition to water) selected from the following list: a solubilizing agent or solvent (e.g., a β-cyclodextrin, such as bydroxypropyl β-cyclodextrin, or an alcohol or polyol such as ethanol, propylene glycol or glycerol); a thickening agent (e.g., hydroxyethylcellulose, hydroxypropylcellulose, carboxymethylcellulose or carbomer); a gelling agent (e.g., a polyoxyethylene-polyoxypropylene copolymer); a preservative (e.g., benzyl alcohol, benzalkonium chloride, chlorhexidine, chlorbutol, a benzoate, potassium sorbate or EDTA or salt thereof); and pH buffering agent(s) (such as a mixture of dihydrogen phosphate and hydrogen phosphate salts, or a mixture of citric acid and a hydrogen phosphate salt). A pharmaceutically acceptable excipient can also be incorporated in a formulation and can be any excipient (e.g., carrier) conventionally used in the art. Non-limiting examples include water, lower alcohols, higher alcohols, polyhydric alcohols, monosaccharides, disaccharides, polysaccharides, hydrocarbon oils, fats and oils, waxes, fatty acids, silicone oils, nonionic surfactants, ionic surfactants, silicone surfactants, and water-based mixtures and emulsion-based mixtures of such carriers.

Other non-limiting examples of pharmaceutical excipients include: antiadherents (e.g., magnesium stearate), binders (e.g., sucrose, lactose, starches, cellulose, microcrystalline cellulose, hydroxypropyl cellulose), sugar alcohols (e.g., xylitol, sorbitol, mannitol), protein (e.g., gelatin), synthetic polymers (e.g., polyvinylpyrrolidone, polyethylene glycol), coatings (e.g., hydroxypropyl methylcellulose, shellac, corn protein zein); disintegrants (e.g., crosslinked sodium carboxymethyl cellulose), starch (e.g., glycolate), glidants (e.g., silica gel, fumed silica, talc, magnesium carbonate), preservatives (e.g., vitamin A, vitamin E, vitamin C, retinyl palmitate, selenium, cysteine, methionine, citric acid, sodium citrate, methyl paraben, propyl paraben), and vehicles (e.g., petrolatum, dimethyl sulfoxide, mineral oil).

Pharmaceutically acceptable diluents or carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethyl alcohol, propyl alcohol, and benzyl alcohol), polyols (e.g., glycerol, propylene glycol, and polyethylene glycol), organic esters (e.g., ethyl oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable diluent or carrier used in a pharmaceutical composition of the disclosure must be “acceptable” in the sense of being compatible with the other ingredients of the formulation and not injurious to the subject. Diluents or carriers suitable for a selected dosage form and intended route of administration are well known in the art, and acceptable diluents or carriers for a chosen dosage form and method of administration can be determined using ordinary skill in the art A film former, when it dries, forms a protective film over the site of application. The film inhibits removal of the active ingredient and keeps it in contact with the site being treated. An example of a film former that is suitable for use herein is Flexible Collodion, USP. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former can act additionally as a carrier. Solutions that dry to form a film are sometimes referred to as paints. Creams, as is well known in the arts of pharmaceutical formulation, are viscous liquids or semisolid emulsions, either oil-in-water or water-in-oil.

Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic or amphoteric surfactant. Lotions are preparations to be applied to the skin surface without friction, and are typically liquid or semiliquid preparations in which particles, including the active agent, are present in a water or alcohol base. Lotions are usually suspensions of solids, and in some embodiments, comprise a liquid oily emulsion of the oil-in-water type. Lotions may be used for treating large body areas, because of the ease of applying a more fluid composition. Lotions, in some embodiments, contain suspending agents to produce better dispersions as well as compounds useful for localizing and holding the active agent in contact with the skin, e.g., methylcellulose, sodium carboxymethylcellulose, or the like.

Solutions are homogeneous mixtures prepared by dissolving one or more chemical substances (solutes) in a liquid such that the molecules of the dissolved substance are dispersed among those of the solvent. A solution can contain other pharmaceutically or cosmetically acceptable chemicals to buffer, stabilize or preserve the solute. Common examples of solvents used in preparing solutions are ethanol, water, propylene glycol or any other acceptable vehicles.

As is of course well known, gels are semisolid, suspension-type systems. Single-phase gels contain organic macromolecules distributed substantially uniformly throughout the carrier liquid, which is typically aqueous, but also, preferably, contain an alcohol, and, optionally, an oil. Example organic macromolecules, e.g., gelling agents, are cross-linked acrylic acid polymers, such as the carbomer family of polymers, e.g., carboxypolyalkylenes that can be obtained commercially under the CARBOPOL® trademark. Other examples are hydrophilic polymers, such as polyethylene oxides, polyoxyethylene-polyoxypropylene copolymers and polyvinylalcohol; cellulosic polymers such as hydroxy-propyl cellulose, hydroxyethyl cellulose, hydroxypropyl methylcellulose, hydroxy-propyl methylcellulose phthalate, and methylcellulose; gums, such as tragacanth and xanthan gum; sodium alginate; and gelatin. In order to prepare a uniform gel, dispersing agents, such as alcohol or glycerin, can be added, or the gelling agent can be dispersed by trituration, mechanical mixing or stirring, or combinations thereof. Ointments, as also well known in the art, are semisolid preparations that are typically based on petrolatum or other petroleum derivatives. The specific ointment base to be used, as will be appreciated by those skilled in the art, is one that will provide for a number of desirable characteristics, e.g., emolliency or the like. As with other carriers or vehicles, an ointment base should be inert, stable, nonirritating, and nonsensitizing. As explained in Remington: The Science and Practice of Pharmacy, 19th Ed. (Easton, Pa.: Mack Publishing Co., 1995), at pages 1399-1404, ointment bases can be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum.

Emulsifiable ointment bases, also known as absorbent ointment bases, contain little or no water and include, for example, hydroxystearin sulfate, anhydrous lanolin, and hydrophilic petrolatum. Emulsion ointment bases are either water-in-oil (W/O) emulsions or oil-in-water (O/W) emulsions, and include, for example, acetyl alcohol, glyceryl monostearate, lanolin, and stearic acid. Preferred water-soluble ointment bases are prepared from polyethylene glycols of varying molecular weight; see Remington: The Science and Practice of Pharmacy, for further information.

Pastes are semisolid dosage forms in which the active agent is suspended in a suitable base. Depending on the nature of the base, pastes are divided between fatty pastes or those made from single-phase aqueous gels. The base in a fatty paste is generally petrolatum or hydrophilic petrolatum or the like. The pastes made from single-phase aqueous gels generally incorporate carboxymethylcellulose or the like as a base.

Enhancers are those lipophilic co-enhancers typically referred to as plasticizing enhancers, e.g., enhancers that have a molecular weight in the range of 150 to 1000, an aqueous solubility of less than 1 wt. %, less than 0.5 wt. %, or less than 0.2 wt. %. The Hildebrand solubility parameter (6) of plasticizing enhancers is in the range of 2.5 to 10, or in the range of 5 to 10. Examples of lipophilic enhancers include fatty esters, fatty alcohols, and fatty ethers. Examples of specific fatty acid esters include methyl laurate, ethyl oleate, propylene glycol propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C2-C4 alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, eds. Smith et al. (CRC Press, 1995).

Various other additives can be included in the compositions of the present disclosure in addition to those identified above. These include, but are not limited to, antioxidants, astringents, perfumes, preservatives, emollients, pigments, dyes, humectants, propellants, and sunscreen agents, as well as other classes of materials whose presence can be pharmaceutically or otherwise desirable. Typical examples of optional additives for inclusion in formulations of the present disclosure are as follows: preservatives, such as sorbate; solvents, such as isopropanol and propylene glycol; astringents, such as menthol and ethanol; emollients, such as polyalkylene methyl glucosides; humectants, such as glycerine; emulsifiers, such as glycerol stearate, PEG-100 stearate, polyglyceryl-3 hydroxylauryl ether, and polysorbate 60; sorbitol and other polyhydroxyalcohols, such as polyethylene glycol; sunscreen agents, such as octyl methoxyl cinnamate (available commercially as Parsol MCX) and butyl methoxy benzoylmethane (available under the tradename PARSOL® 1789); antioxidants such as ascorbic acid (vitamin C), α-tocopherol (Vitamin E), β-tocopherol, γ-tocopherol, δ-tocopherol, ε-tocopherol, ζ₁-tocopherol, ZΓ-tocopherol, η-tocopherol, and retinol (vitamin A); essential oils, ceramides, essential fatty acids, mineral oils, vegetable oils (e.g., soya bean oil, palm oil, liquid fraction of shea butter, sunflower oil), animal oils (e.g., perhydrosqualene), synthetic oils, silicone oils or waxes (e.g., cyclomethicone and dimethicone), fluorinated oils (generally perfluoropolyethers), fatty alcohols (e.g., cetyl alcohol), and waxes (e.g., beeswax, carnauba wax, and paraffin wax); skin-feel modifiers; and thickeners and structurants such as swelling clays and cross-linked carboxypolyalkylenes that can be obtained commercially under the CARBOPOL® trademark. Other additives include beneficial agents such as those materials that condition the skin (particularly, the upper layers of the skin in the stratum corneum) and keep it soft by retarding the decrease of its water content and/or protect the skin. Such conditioners and moisturizing agents include, by way of example, pyrrolidine carboxylic acid and amino acids; organic antimicrobial agents such as 2,4,4′-trichloro-2-hydroxy diphenyl ether (triclosan) and benzoic acid; anti-inflammatory agents such as acetylsalicylic acid and glycyrrhetinic acid; anti-seborrhoeic agents such as retinoic acid; vasodilators such as nicotinic acid; inhibitors of melanogenesis such as kojic acid; and mixtures thereof. Further additional active agents including, for example, alpha hydroxyacids, alpha ketoacids, polymeric hydroxyacids, moisturizers, collagen, marine extract, and antioxidants, and/or pharmaceutically acceptable salts, esters, amides, or other derivatives thereof. Additional agents include those that are capable of improving oxygen supply in skin tissue. Sunscreens and UV absorbing compounds can also be included. Non-limiting examples of such sunscreens and UV absorbing compounds include aminobenzoic acid (PABA), avobenzone, cinoxate, dioxybenzone, homosalate, menthyl anthranilate, oxtocrylene, octyl methoxycmnamate, octyl salicylate, oxybenzone, padirnate O, phenylbenzirmdazole sulfonic acid, sulisobenzone, titanium dioxide, trolamine salicylate, zinc oxide, ensulizole, meradiraate, octinoxate, octisalate, and octocrylene.

Other embodiments can include a variety of non-carcinogenic, non-irritating healing materials that facilitate treatment with the formulations of the disclosure. Such healing materials can include nutrients, minerals, vitamins, electrolytes, enzymes, herbs, plant extracts, glandular or animal extracts, or safe therapeutic agents that can be added to the formulation to facilitate the healing of dermal disorders.

The amounts of these various additives are those conventionally used in the cosmetics field, and range, for example, from 0.01% to 20% of the total weight of the topical formulation. Formulations of the present disclosure can also include conventional additives such as opacifiers, fragrance, colorant, stabilizers, surfactants, and the like. In some embodiments, other agents can also be added, such as antimicrobial agents, to prevent spoilage upon storage, i.e., to inhibit growth of microbes such as yeasts and molds.

Suitable antimicrobial agents are typically selected from the group consisting of the methyl and propyl esters of p-hydroxybenzoic acid (methyl and propyl paraben), sodium benzoate, sorbic acid, imidurea, and combinations thereof. In other embodiments, other agents can also be added, such as repressors and inducers, e.g., to inhibit (glycose) or induce (xylose) the production of the polypeptide of interest. Such additives can be employed provided they are compatible with and do not interfere with the function of the formulations.

Formulations can also contain irritation-mitigating additives to minimize or eliminate the possibility of skin irritation or skin damage resulting from the chemical entity to be administered, or other components of the composition.

Suitable irritation-mitigating additives include, for example: a-tocopherol; monoamine oxidase inhibitors, particularly phenyl alcohols such as 2-phenyl-1-ethanol; glycerin; salicylates; ascorbates; ionophores such as monensin; amphophilic amines; ammonium chloride; N-acetylcysteine; capsaicin; and chloroquine. The irritation-mitigating additive, if present, can be incorporated into the compositions at a concentration effective to mitigate irritation or skin damage, typically representing not more than 20 wt. %, more typically not more than 5 wt. %, of the formulation.

Further suitable pharmacologically active agents that can be incorporated into the present formulations in some embodiments and thus topically applied along with the admixture of S. epidermidis cells include, but are not limited to, the following: agents that improve or eradicate pigmented or non-pigmented age spots, keratoses, and wrinkles; antimicrobial agents; antibacterial agents; antipruritic and antixerotic agents; anti-inflammatory agents; local anesthetics and analgesics; corticosteroids; retinoids; vitamins; hormones; and antimetabolites.

Some examples of topical pharmacologically active agents include acyclovir, amphotericins, chlorhexidine, clotrimazole, ketoconazole, econazole, miconazole, metronidazole, minocycline, nystatin, neomycin, kanamycin, phenytoin, para-amino benzoic acid esters, octyl methoxycmnamate, octyl salicylate, oxybenzone, dioxybenzone, tocopherol, tocopheryl acetate, selenium sulfide, zinc pyrithione, diphenhydramine, pramoxine, lidocaine, procaine, erythromycin, tetracycline, clindamycin, crotamiton, hydroquinone and its monomethyl and benzyl ethers, naproxen, ibuprofen, cromolyn, retinol, retinyl palmitate, retinyl acetate, coal tar, griseofulvin, estradiol, hydrocortisone, hydrocortisone 21-acetate, hydrocortisone 17-valerate, hydrocortisone 17-butyrate, progesterone, betamethasone valerate, betamethasone dipropionate, triamcinolone acetonide, fluocinonide, clobetasol propionate, minoxidil, dipyridamole, diphenylhydantoin, benzoyl peroxide, and 5-fluorouracil.

A cream, lotion, gel, ointment, paste or the like can be spread on the affected surface and gently rubbed in. A solution can be applied in the same way, but more typically will be applied with a dropper, swab, or the like, and carefully applied to the affected areas.

The application regimen will depend on a number of factors that can readily be determined, such as the severity of the condition and its responsiveness to initial treatment, but will normally involve one or more applications per day on an ongoing basis. One of ordinary skill can readily determine the optimum amount of the formulation to be administered, administration methodologies and repetition rates. In general, it is contemplated that the formulations of the disclosure will be applied in the range of once or twice weekly up to once or twice daily.

### Methods of Treating or Preventing Disease

In some aspects, the present disclosure provides a method of treating or preventing skin disease in a subject by administering to the subject a composition provided herein, e.g., comprising an admixture of S. epidermidis cells. In some embodiments, the composition modifies expression levels of nitrogen respiration genes, urease activity genes, carbohydrate metabolism genes, iron uptake genes, sulfur metabolism genes, and/or virulence factor genes. Skin diseases may be any skin diseases associated with S. epidermidis. Non-limiting examples of skin diseases associated with S. epidermidis include: folliculitis, furuncles, carbuncles, impetigo, echtyma, cellulitis, atopic dermatitis, and folliculitis declavans.

In some embodiments, a skin disease associated with S. epidermidis is folliculitis. Folliculitis is a common skin condition in which hair follicles become inflamed as a result of bacterial or fungal infection. Symptoms of folliculitis include groups of small red bumps capped with white, blisters to break, large areas of red, swollen skin that may leak pus, itchy skin, tender skin, and painful skin.

In some embodiments, a skin disease associated with S. epidermidis is a furuncle (boil). Furuncles are deep infections of the hair follicle that lead to abscess formation, accumulation of pus, and formation of necrotic tissue. Symptoms of a furuncle include red, swollen and tender nodules of varying size with an optional overlapping pustule; fever; and enlarged lymph nodes.

In some embodiments, a skin disease associated with S. epidermidis is a carbuncle. Carbuncles are clusters of furuncles that are typically filled with pus and may develop anywhere on the body. Symptoms of carbuncles include red, swollen lumps that later soften at the center and discharge pus, itching, localized erythema, skin irritation, pain upon touching, fatigue, fever, chills, and general malaise.

In some embodiments, a skin disease associated with S. epidermidis is impetigo. Impetigo is a highly contagious bacterial infection involving the superficial skin. Symptoms of impetigo include yellowish crusts on the face, arms, or legs; large blisters in the groin or armpit; fever; and painful or itchy skin.

In some embodiments, a skin disease associated with S. epidermidis is ecthyma. Ecthyma is a deep form of impetigo which causes deeper erosions of the skin that stretch into the dermis. Ecthyma most often occurs on the buttocks, thighs, legs, ankles, and feet. Symptoms of ecthyma include indurated ulcers, swollen lymph nodes, pain upon touching, fever, and general malaise.

In some embodiments, a skin disease associated with S. epidermidis is cellulitis. Cellulitis is a bacterial infection involving the deeper layers of the skin, including the dermis and the subcutaneous fat. Cellulitis commonly occurs on the legs and face, although any part of the body may be infected. Symptoms of cellulitis include an area of redness which increases in size over a few days; swollen skin, pain upon touching; fever; and general malaise.

In some embodiments, a skin disease associated with S. epidermidis is atopic dermatitis (eczema). In children under one year of age, much of the body is affected, whereas older children tend to be affected on the back of the knees and the front of the elbows and adults tend to be affected on the hands and feet. Atopic dermatitis is an inflammation of the skin that results in itchy, red, swollen, and cracked skin that may leak clear fluid. Symptoms of atopic dermatitis include dry and scaly skin that covers the body and intensely itchy red, splotchy lesions in the bends of the arms or legs, face, and neck.

In some embodiments, a skin disease associated with S. epidermidis is folliculitis declavans. Folliculitis declavans is an infection and inflammation of the hair follicle. Symptoms of folliculitis declavans include indurations of the scalp along with pustules, erosions, crusts, ulcers, and scale. These indurations begin at a central point and spread outwards, leading to scarring, sores, and hair loss.

In some embodiments, a skin disease associated with Netherton Syndrome. Netherton syndrome is a disorder that affects the skin, hair, and immune system. Newborns with Netherton syndrome have skin that is red and scaly (ichthyosiform erythroderma), and the skin may leak fluid.

In some embodiments, the admixtures and compositions provided herein may be used to treat, prevent, and/or reduce the risk of bloodstream (e.g., due to catheter) infection by S. epidermidis.

An admixture of S. epidermidis cells may be administered by any route known in the art. Non-limiting examples of routes of administration include: topical, transdermal, oral (liquid or solid), intravenous, intramuscular, sublingual, buccal, and nasal.

A subject that is administered an admixture of S. epidermidis cells of the present disclosure may be any subject in need thereof. This subject may have a skin disease that is associated with S. epidermidis. In some embodiments, the subject has or is at risk for having a skin disease (e.g., folliculitis, furuncles, carbuncles, impetigo, echtyma, cellulitis, atopic dermatitis, folliculitis declavans, and Netherton syndrome). In some embodiments, the subject is a mammal, such as a human. Other mammals are contemplated herein.

A subject, in some embodiments, is a mammal. Non-limiting examples of mammals include humans, farm animals, domestic animals, laboratory animals, etc. Some examples of farm animals include cows, pigs, horses, goats, etc. Some examples of domestic animals include dogs, cats, etc. Some examples of laboratory animals include primates, rats, mice, rabbits, guinea pigs, etc. In some embodiments, the mammal is a human.

The term “treat” (and variations thereof) means providing to a subject a protocol, regimen, process or remedy, in which it is desired to obtain a physiologic response or outcome in that subject, e.g., a patient. In particular, the methods and compositions of the present disclosure may be used to slow the development of skin disease symptoms (e.g., associated with S. epidermidis infection) or delay the onset of the disease, or halt the progression of disease development. Because every treated subject may not respond to a particular treatment protocol, regimen, process or remedy, treating does not require that the desired physiologic response or outcome be achieved in each and every subject or subject population. Accordingly, a given subject may fail to respond or respond inadequately to treatment.

In some embodiments, treatment slows the development of skin disease symptoms or delay the onset of the disease or halt the progression of disease development of skin diseases associated with S. epidermidis by 50%-100%, compared to control. In some embodiments, treatment slows the development of skin disease symptoms or delay the onset of the disease or halt the progression of disease development of skin diseases associated with S. epidermidis by 60%-95%. In some embodiments, treatment slows the development of skin disease symptoms or delay the onset of the disease or halt the progression of disease development of skin diseases associated with S. epidermidis by 70%-80%. In some embodiments, treatment slows the development of skin disease symptoms or delay the onset of the disease, or halt the progression of disease development of skin diseases associated with S. epidermidis by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

The term “prevent” (and variations thereof) means providing to a subject a protocol, regimen, process or remedy, in which it is desired to forestall a skin disease in that subject, e.g., a patient. In particular, the methods and admixtures of the present disclosure may be used to prevent the development of skin disease symptoms (e.g., associated with S. epidermidis infection). Because every treated subject may not respond to a particular prevention protocol, regimen, process or remedy, preventing does not require that the desired physiologic response or outcome be achieved in each and every subject or subject population. Accordingly, a given subject may fail to respond or respond inadequately to prevention.

In some embodiments, prevention forestalls skin diseases associated with S. epidermidis by 50%-100%, compared to control. In some embodiments, prevention forestalls skin diseases associated with S. epidermidis by 60%-95%. In some embodiments, prevention forestalls skin diseases associated with S. epidermidis by 70%-80%. In some embodiments, prevention forestalls skin diseases associated with S. epidermidis by 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

An admixture of the present disclosure is administered to a subject in need thereof at an effective amount. An effective amount (also referred to as a therapeutically effective amount) of an admixture of S. epidermidis cells that is sufficient to effect a beneficial or desired results as described herein when administered to a subject (e.g., ameliorate a symptom of a skin disease). Effective dosage forms, modes of administration, and dosage amounts may be determined empirically, and making such determinations is within the skill of the art. It is understood by those skilled in the art that the dosage amount will vary with the route of administration, the rate of excretion, the duration of the treatment, the identity of any other drugs being administered, the age, size, and species of mammal, e.g., human patient, and like factors well known in the arts of medicine and veterinary medicine. In general, a suitable dose of an admixture of S. epidermidis cells is the lowest dose effective to produce the desired effect. The effective dose of an admixture of S. epidermidis cells may be administered as two, three, four, five, six or more sub-doses, administered separately at appropriate intervals throughout the day, for example.

An effective amount of an admixture of S. epidermidis cells may be any amount that modifies the gene expression of nitrogen respiration genes, urease activity genes, carbohydrate metabolism genes, iron uptake genes, sulfur metabolism genes and/or virulence factor genes in the skin cells compared to control cells.

In some embodiments, the effective amount has a total colony forming unit (cfu) concentration of 10⁶-10¹⁴ cfu/millilieter (cfu/mL). In some embodiments, the effective amount has a concentration of 10⁷-10¹² cfu/mL. In some embodiments, the effective amount has a concentration of 108-10¹¹ cfu/mL. In some embodiments, the effective amount has an IC50 of 10⁶ cfu/mL, 10⁷ cfu/mL, 10⁸ cfu/mL, 10⁹ cfu/mL, 10¹⁰ cfu/mL, 10¹¹ cfu/mL, 10¹² cfu/mL, 103 cfu/mL, or 10¹⁴ cfu/mL.

### Methods of Reducing the Risk of Recurrent Skin Disease

In some aspects, the present disclosure provides a method of reducing the risk of recurrent skin disease in a subject by administering to the subject an admixture of S. epidermidis cells that modifies expression levels of nitrogen respiration genes, urease activity genes, carbohydrate metabolism genes, iron uptake genes, sulfur metabolism genes, and/or virulence factor genes. Recurrent skin diseases may be any skin disease associated with S. epidermidis that recurs at least one after symptoms disappear. Causes of recurrent skin diseases may include incomplete decolonization of S. epidermidis, increased subject susceptibility to S. epidermidis infection, and contagious spread of S. epidermidis. Non-limiting examples of recurrent skin disease associated with S. epidermidis include: atopic dermatitis, furuncles, carbuncles, and impetigo. The signs and symptoms of these skin diseases are discussed above.

The term “risk reduction” (and variations thereof) means providing to a subject a protocol, regimen, process or remedy, in which it is desired to forestall a skin disease that has already occurred in that subject, e.g., a patient. In particular, the methods and admixtures of the present disclosure may be used to reduce the risk of the development of skin disease symptoms that have already occurred in the subject. Because every treated subject may not respond to a particular risk reduction protocol, regimen, process or remedy, risk reduction does not require that the desired physiologic response or outcome be achieved in each and every subject or subject population, or that the desired physiologic response be completely absent in the subject. Accordingly, a given subject may fail to respond or respond inadequately to risk reduction.

In some embodiments, risk reduction reduces the recurrence of skin diseases associated with S. epidermidis by 50% (e.g., half as many recurrences as control)−100% (e.g., no recurrences compared to control). In some embodiments, risk reduction reduces the recurrence of skin diseases associated with S. epidermidis by 60%-95%. In some embodiments, risk reduction reduces the recurrence of skin diseases associated with S. epidermidis by 70%-80%. In some embodiments, risk reduction reduces the recurrence of skin diseases associated with S. epidermidis by at least 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%.

A control subject may be a subject with the same history of S. epidermidis associated skin diseases that is not risk reduced or the same subject before risk reduction.

### EXAMPLES

### Example 1: Skin Staphylococcus epidermidis (S. epidermidis) Population Diversity is Shaped by Transmission and Skin Site Specialization

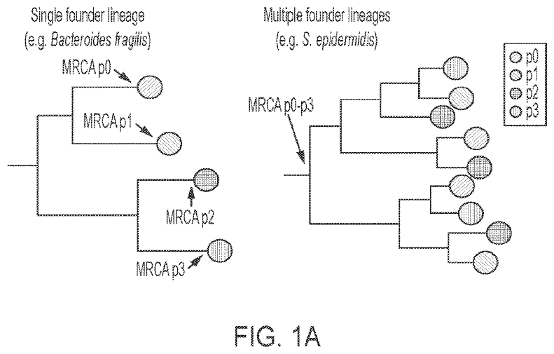

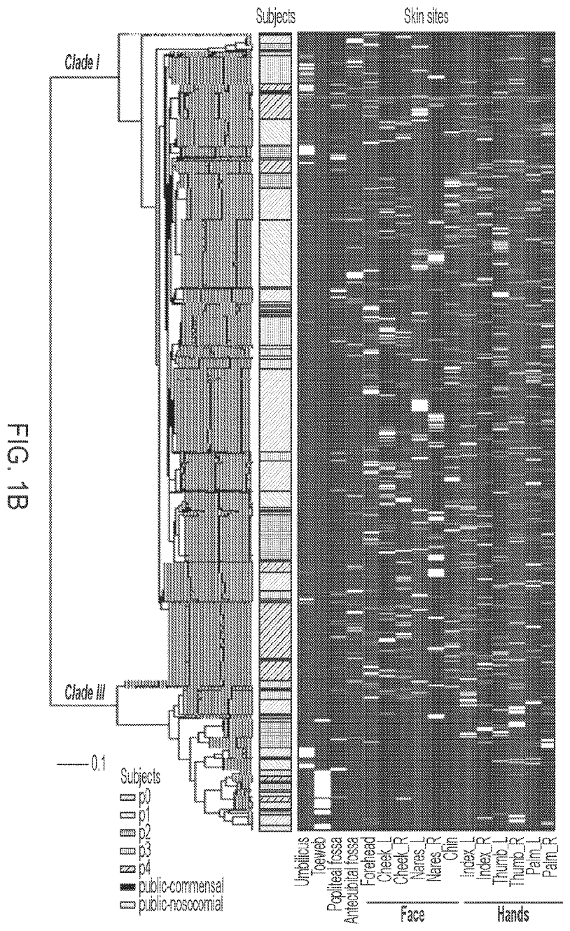

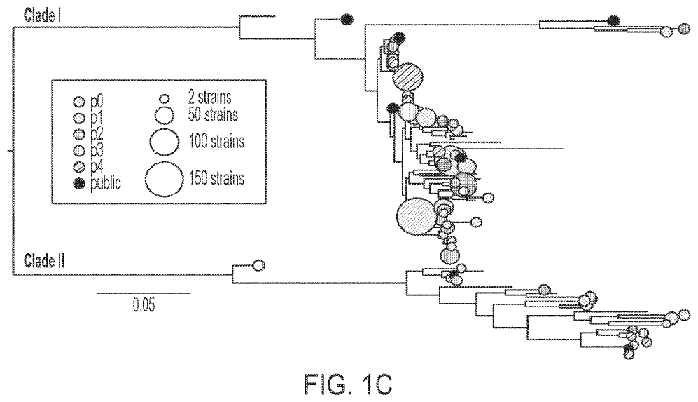

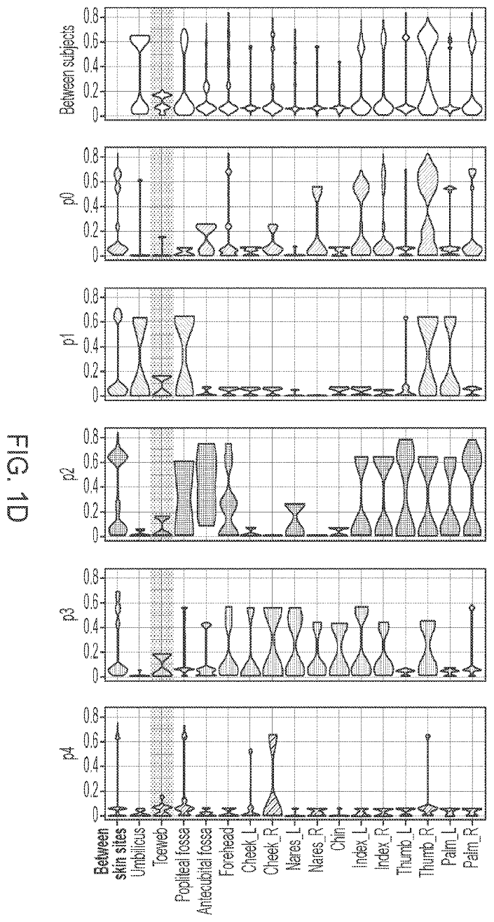

Microbial populations on the human skin can be derived from a single founding member over one's lifetime in the absence of major perturbations (a single colonizer hypothesis), or alternatively colonized by multiple founder lineages. These hypothetical processes are distinguishable depending on whether isolates sampled from different individuals have distinct most recent common ancestors (MRCAs)—suggesting a single colonization event in each individual, such as that observed for Bacteroides fragilis in the human gut (Zhao et al., 2019)—or not, suggesting the presence of multiple founder lineages (FIG. 1A). Here, we reconstructed the molecular phylogeny based on SNPs in the core genome (regions conserved across all isolates) from our 1482 subject isolates (data not shown) and 50 publicly deposited, high-quality S. epidermidis genomes sampled from healthy and diseased individuals (Conlan et al., 2012 and the unpublished VCU collection, data not shown). Collectively, S. epidermidis isolates formed two major phylogenetic clades (FIG. 1B), similar to previous observations (Conlan et al., 2012; Oh et al., 2014). Surprisingly, isolates from each subject, as well as the 50 public isolates, shared a MRCA, suggesting the presence of multiple founder lineages (FIGS. 1B and 1C). This observation revealed that unlike B. fragilis, diversity within founder lineages is maintained in S. epidermidis, resulting in broad phylogenetic representation within a single host.

Moreover, subject isolates exhibited subject- and skin site-specific phylogenetic structuring (FIG. 1D, PERMANOVA based on cophenetic distances, p=0.001 for skin sites, subjects, and their interaction term). Strikingly, all toeweb isolates spanned a limited phylogenetic space not only within each of the five subjects, but also between subjects (FIG. 1D). Such uniformity in phylogenetic similarity of toeweb isolates was unlikely due to stochastic mechanisms (e.g., population bottleneck), suggesting a stronger purifying selection favoring the growth of a particular genetic configuration.

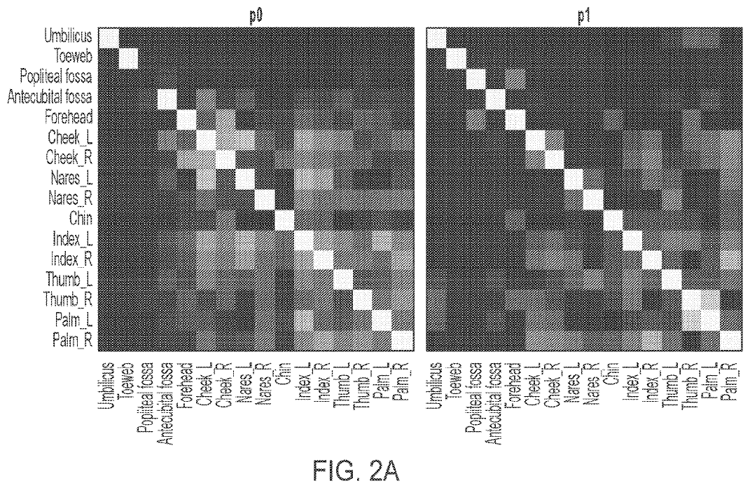

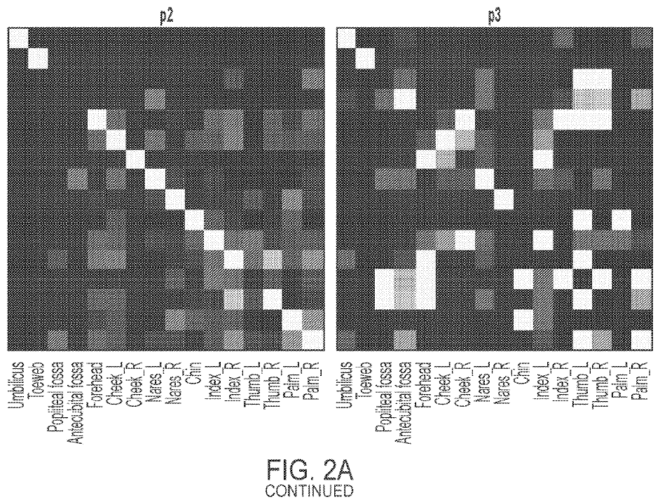

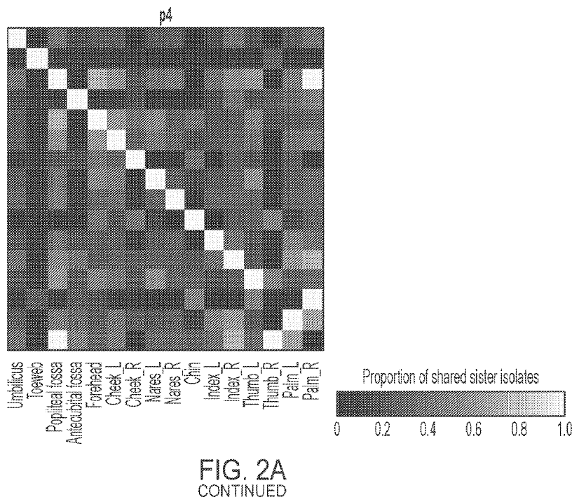

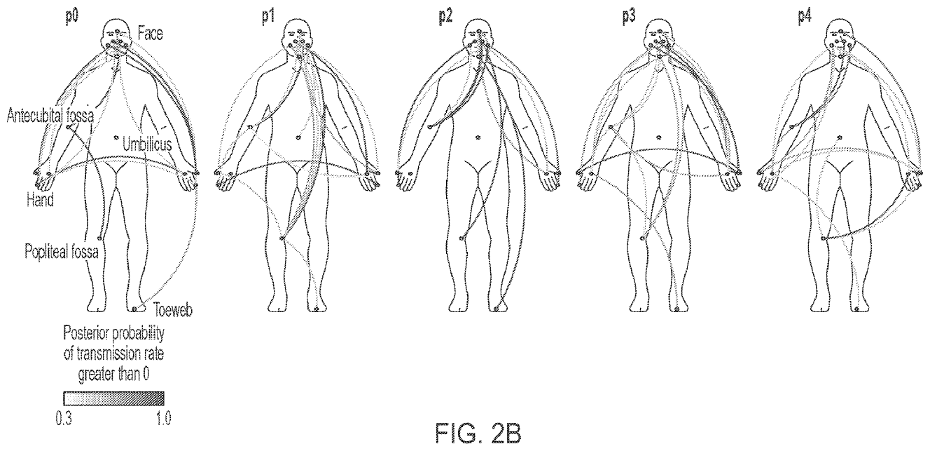

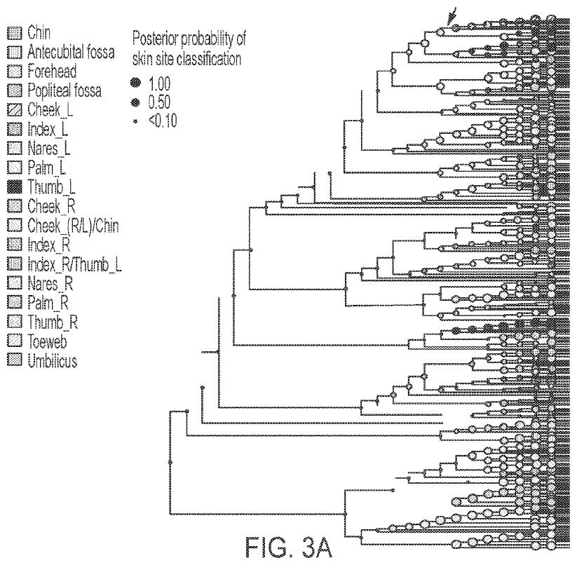

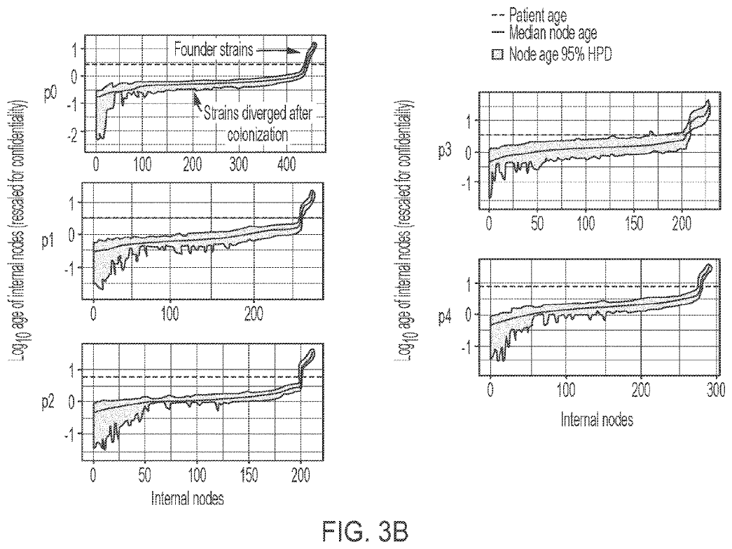

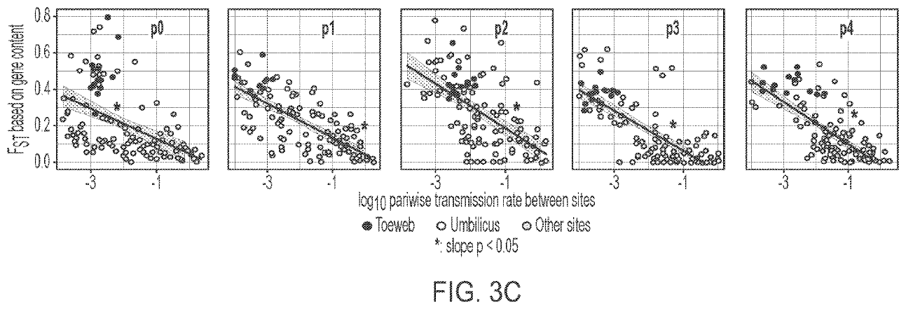

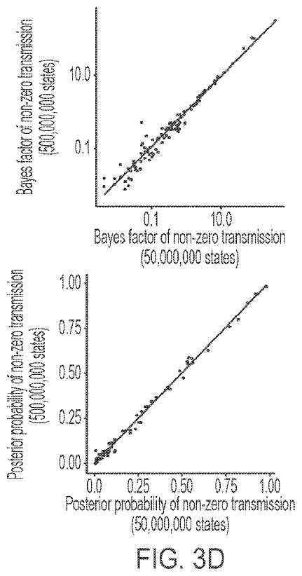

An evolutionary process particularly relevant to skin microbes is transmission of bacteria across environmental barriers, which can further modulate the genetic diversity landscape (Brito and Alm, 2016; Mideo et al., 2008; Niehus et al., 2015). Indeed, sister isolates-isolates with zero core-genome nucleotide differences, likely representing recent descendants of the same lineage—were observed at different skin sites (FIG. 2A). Although the number of shared sister isolates could be skewed due to sampling depth and the overall frequency of sister isolates within a population, it is highly unlikely for isolates observed at different skin sites to accumulate exactly the same alleles at all core-genome SNP loci (n=58498). Therefore, the presence of shared sister isolates heuristically supports the presence of recent transmission events. However, shared sister isolates cannot be used to infer historical transmissions nor provide quantitative estimates of transmission rate. To quantitatively estimate and compare transmission between skin sites, we inferred transmission events along a phylogenetic tree using Bayesian evolutionary analysis by sampling trees (BEAST) (Suchard et al., 2018). BEAST represented transmission events as switches in node states (for an example, see FIG. 3A), and estimated transmission probability by sampling many time-calibrated phylogenetic trees (n=2000 in this study). Based on the time calibration, isolates from each subject diverged from multiple ancestral nodes that were older than the subject (FIG. 3B) and suggested that the S. epidermidis population on a given subject was likely established by at least 12-20 founder strains (FIG. 3B). The diverse founder strains then further diverged into the isolates observed in this study, at least partially explaining their broad phylogenetic representation (FIG. 1B).

We then estimated the probability of transmission between each pair of skin sites, resulting in a probabilistic transmission map of within-individual S. epidermidis on the human skin (FIG. 2B). Both the transmission map and the previous heuristic method (FIG. 2A) revealed that 1) transmission occurs frequently between facial sites and hand sites, which are relatively exposed, 2) transmission patterns are subject-specific, and 3) the umbilicus and toeweb were relatively isolated from the rest of the skin sites. For the toeweb and umbilicus, the lack of transmission can result in distinct S. epidermidis gene contents in these subpopulations (FIG. 3C). Interestingly, in subject p3, which had relatively few shared sister isolates (FIG. 2A), transmission between facial and hand sites was still frequent (FIG. 2B), consistent with the presence of closely related isolates with few (but not zero) SNPs at different skin sites (FIG. 1D). Biologically, this could be due to larger effective population sizes in p3, which decreased the probability of observing sister isolates shared between skin sites. Note that the heuristic method (FIG. 2A) and the probabilistic transmission map (FIG. 2B) showed some incongruencies for a subset of skin sites; this is because the heuristic method reports sharing of sister isolates “as it is”, while the probabilistic transmission analyses additionally infers the transmission rates that are necessary to explain the data through variable selection (see Example 5, Materials and Methods). Altogether, these findings suggested that topography is an important determinant of S. epidermidis population diversity in the skin and is further shaped by transmission between sites such as the hand and the face, or geographic isolation, as for the umbilicus and toeweb.

### Example 2: Individualized, Skin-Site-Dependent and Dynamic Evolution of S. epidermidis Gene Content

Gene content diversification-likely driven both by transmission and natural selection is important because it indicates the functional capacity of a given isolate, and also how that capacity is constrained within a host, is associated with skin sites, and fluctuates over time. Previous metagenomic studies have suggested host- and skin-site-specificity of strain-specific gene content (Tett et al., 2017); here, we leveraged our large isolate dataset to test these hypotheses.

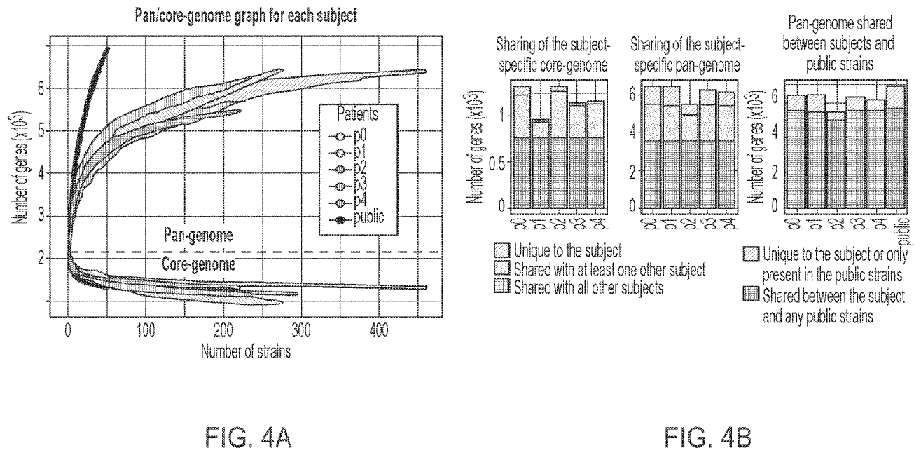

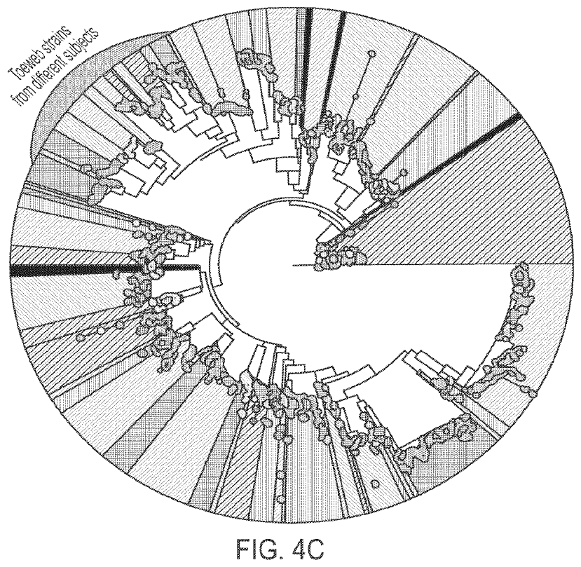

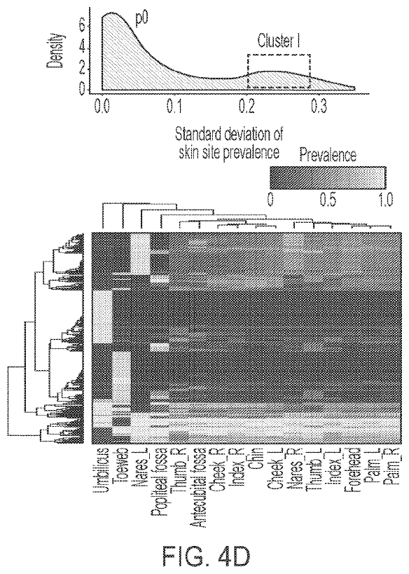

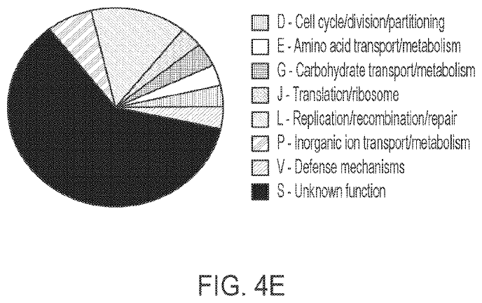

Isolates sampled from each subject constituted relatively closed pan-genomes with comparable sizes across the subjects (FIG. 4A), suggesting a limited repertoire of within-individual gene content. Conversely, this gene content showed considerable subject-specificity. Only 55.4%-65.1% of the S. epidermidis gene clusters observed in a given subject (i.e., the subject-specific pan-genome) and 58.0%-80.5% of the gene clusters observed in all isolates of a given subject (i.e., the subject-specific core-genome) were shared between all five subjects, and 9.5%-15.6% of gene clusters were entirely unique to a subject (FIG. 4B). These findings suggested a personalization of gene content at the population-level: while the S. epidermidis population found in a single host retains the inherent diversity from multiple founder lineages, further evolution of the S. epidermidis gene repertoire occurred in a host-specific manner. Consistent with this observation was the much greater size of the collective pan-genome of the 50 publicly available genomes (FIG. 4A) and additional 17.4% of the gene clusters unique to the public strains (FIG. 4B). Put together, our results showed that, despite their wide distribution in the SNP-based phylogeny (FIG. 1B), S. epidermidis within a single host had constrained gene content diversity.

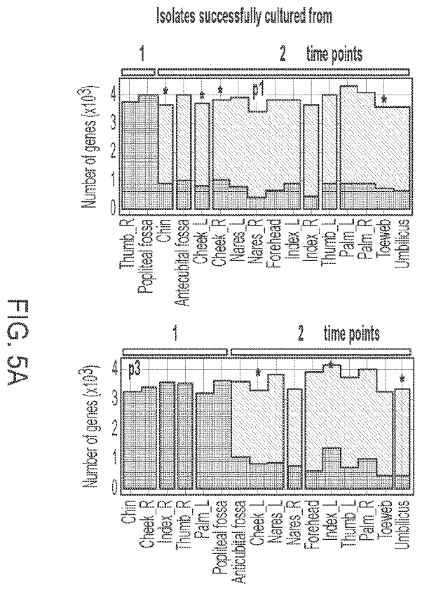

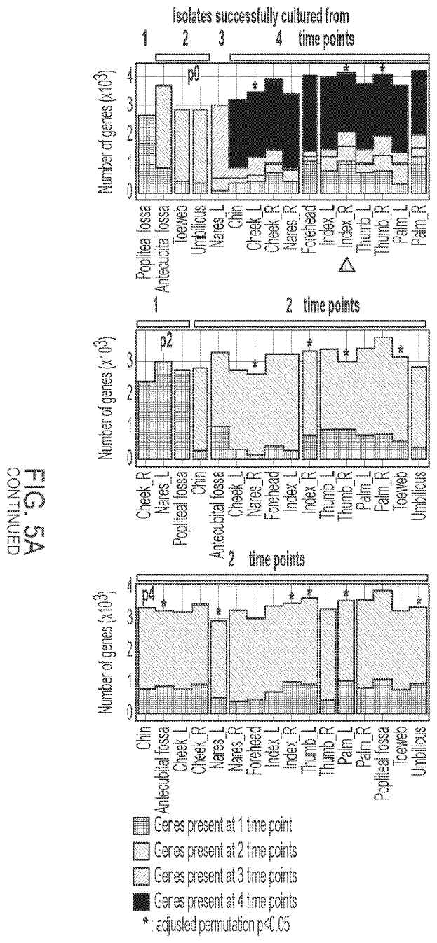

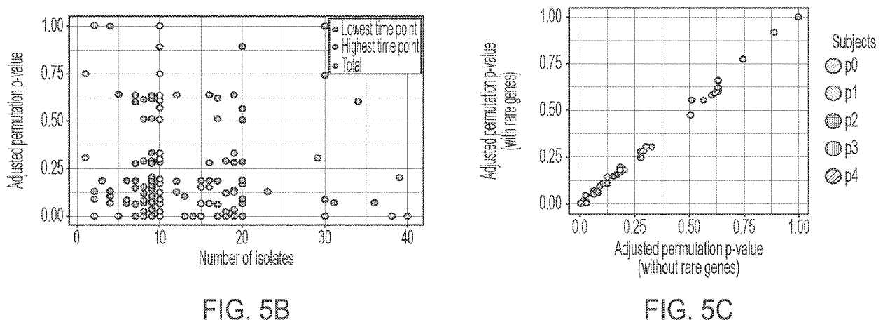

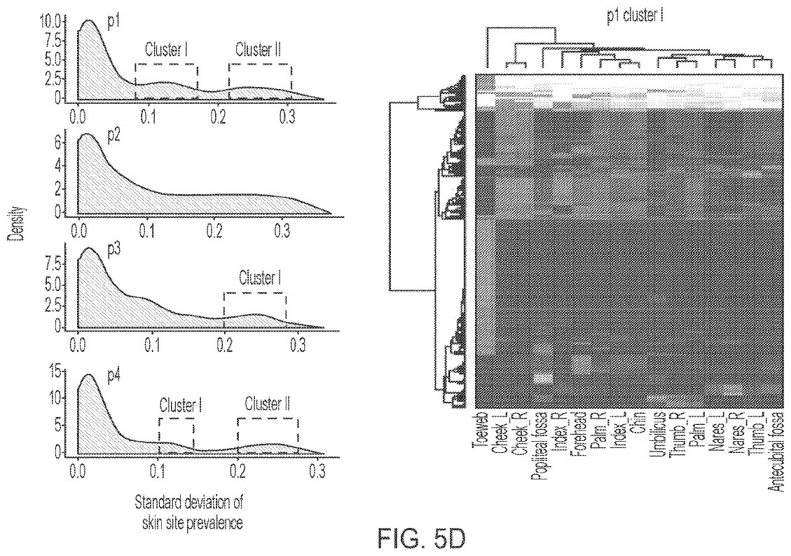

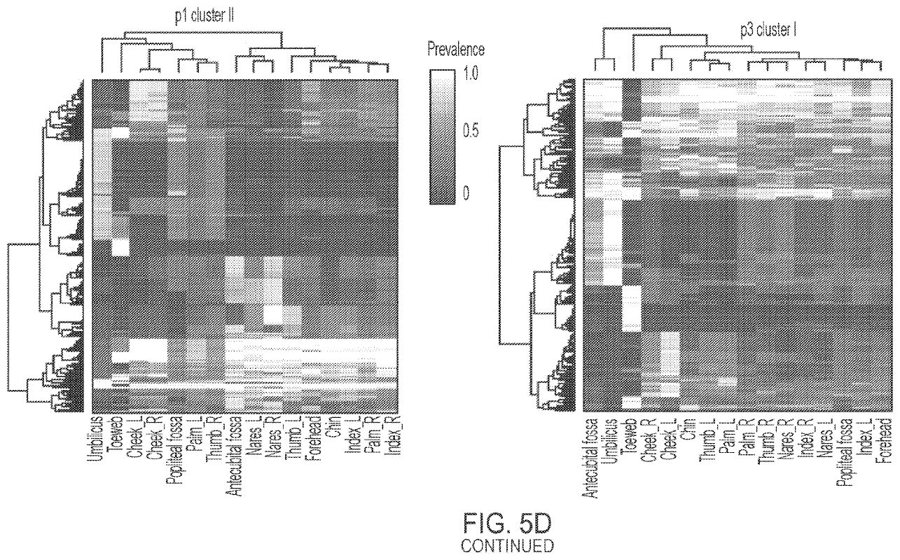

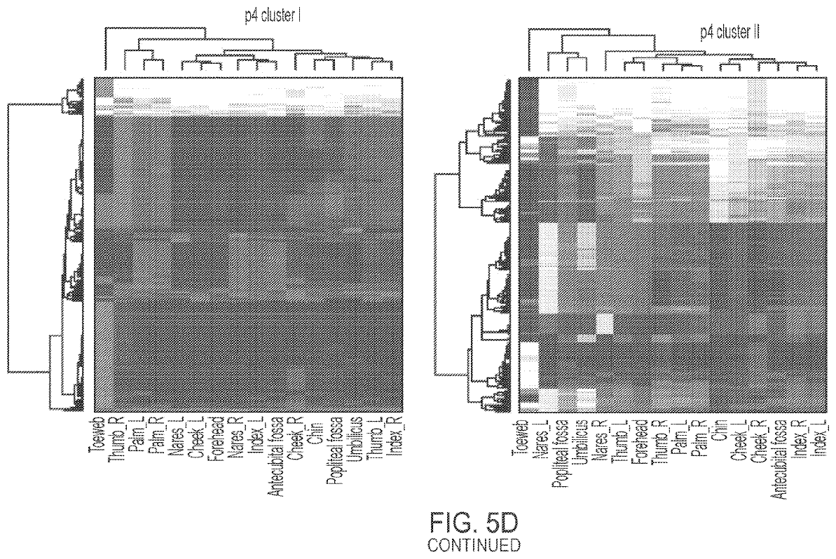

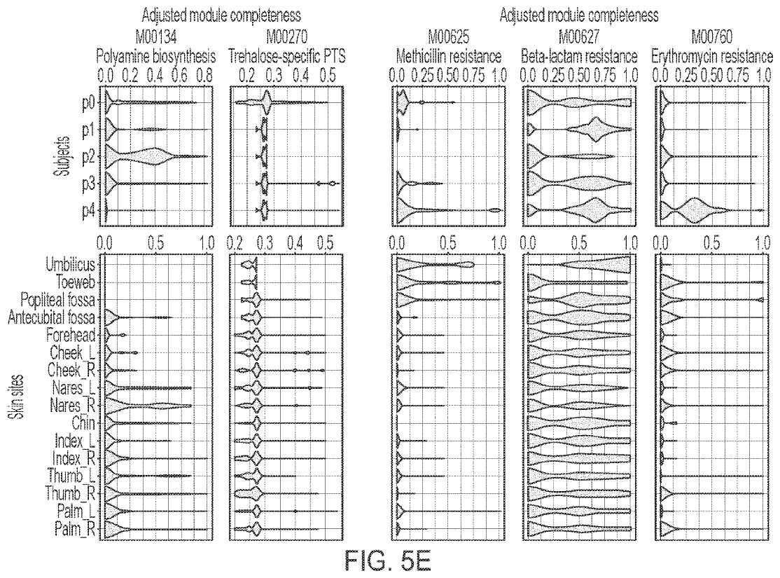

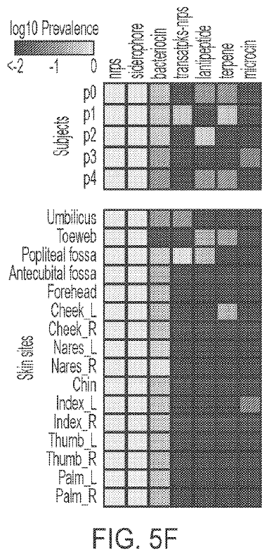

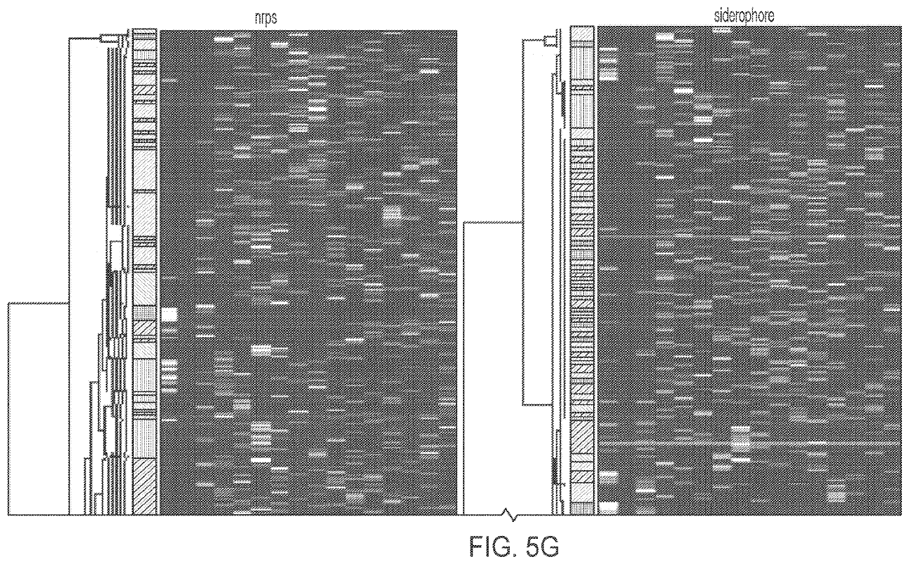

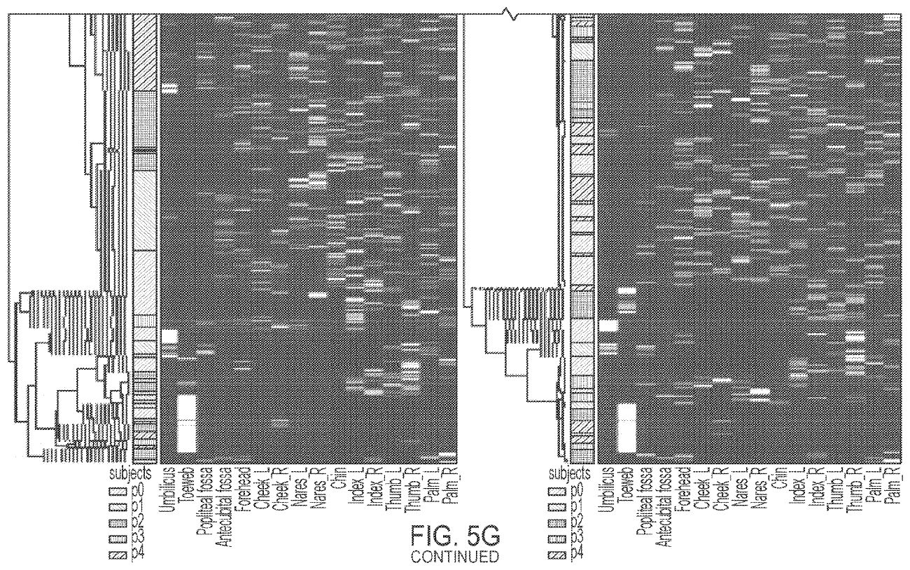

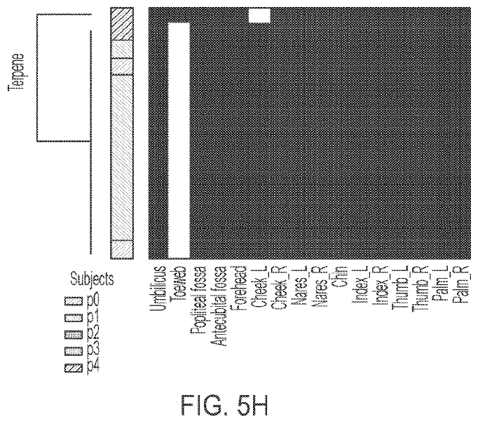

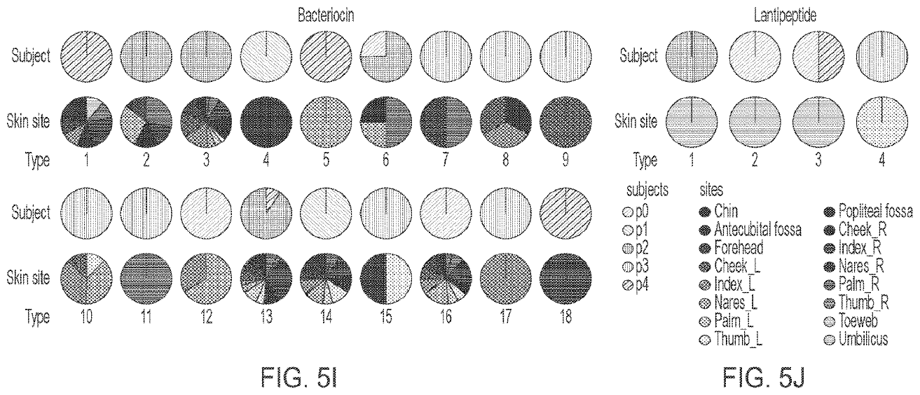

Next, we sought to further dissect the spatio-temporal variation in this individualized gene repertoire. In addition to moderate yet potentially significant temporal fluctuations (FIGS. 5A-5C), S. epidermidis gene content showed structuring by skin site: toeweb isolates consistently contained distinct gene contents compared to isolates from other skin sites, as revealed by hierarchical clustering of the subject isolates based on the presence and absence of accessory genes (FIG. 4C). Genes that were specifically present or absent in the toeweb isolates consistently constituted a substantial fraction of the site-specific accessory genes (i.e., a relatively large standard deviation of prevalence across skin sites, FIGS. 4D and 5D). This suggested a strong specialization of gene content to the toeweb via both entirely unique genes as well as a lack of many genes common to other skin sites (FIGS. 4D and 5D). Lack of transmission also likely contributed to the distinct S. epidermidis gene contents in these subpopulations, which was also observed in the umbilicus (FIGS. 4D and 3C). Yet the biological functions of the toeweb-specific genes were largely obscure (FIGS. 4E and data not shown), underscoring the need for additional tools to study strain-specific gene functions. Other annotatable biological functions, including KEGG modules, lantibiotics, and other biosynthetic gene clusters (BGCs), also showed host-specificity and skin site-heterogeneity (data not shown) in both prevalence (FIGS. 5E-5F) and sequence variation (FIGS. 5G-5J).

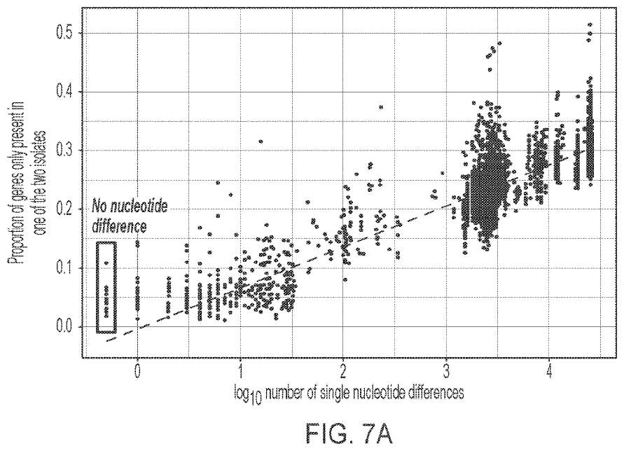

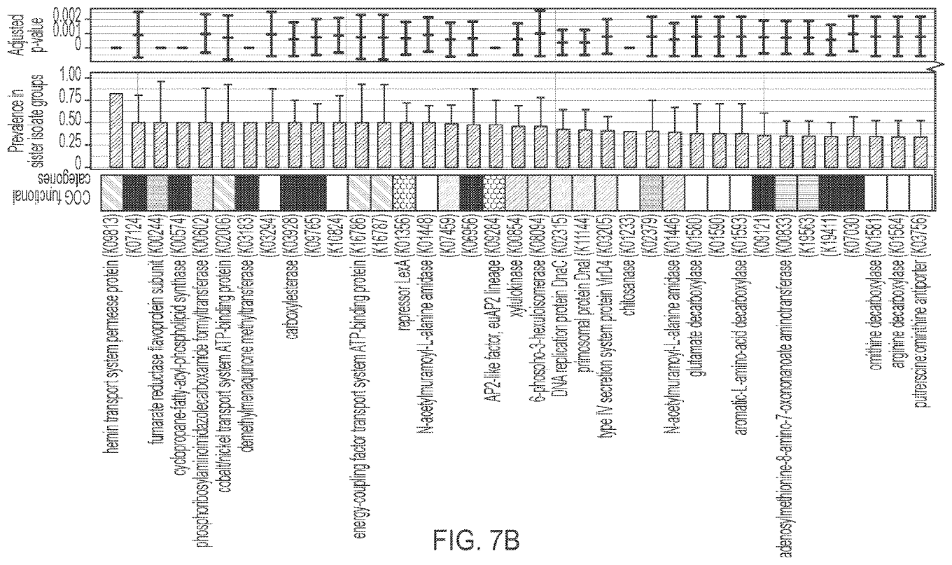

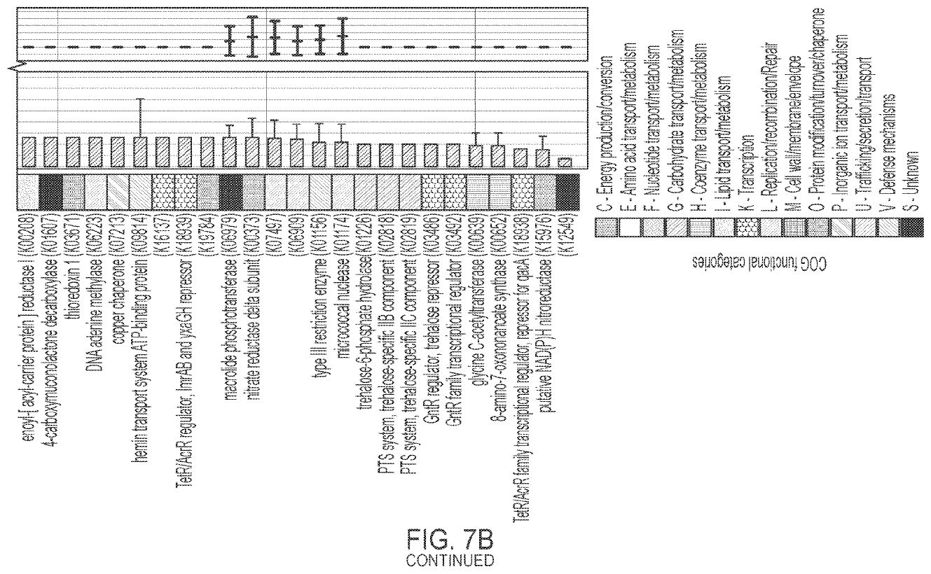

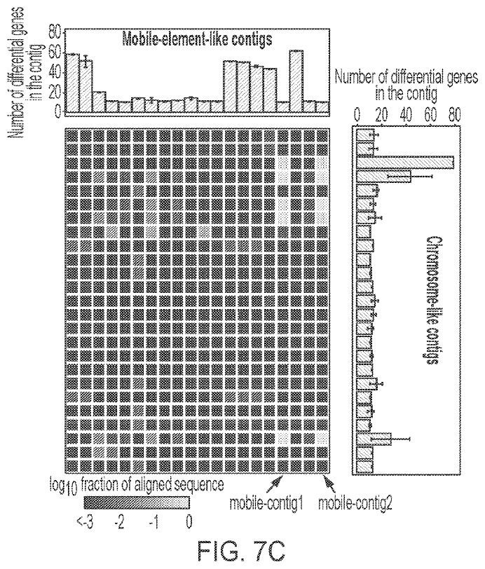

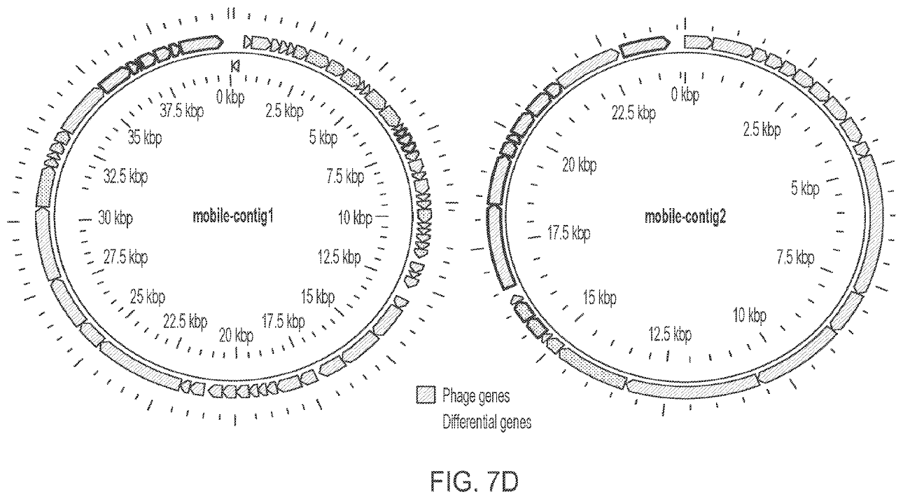

Given this extensive gene-level diversity, we anticipated that isolates with the closest vertical evolutionary history (as assessed by core-genome SNP differences, Duchene et al., 2016) would have the most similar gene content. However, we found a significant incongruence in SNP differences and gene content heterogeneity (FIG. 7A, linear regression R²=0.503): 5.0%±3.2% of genes differed even between sister isolates, which have no core-genome SNP differences, suggesting the presence of very recent evolutionary processes that increase gene content diversity (FIG. 7A). To study such processes, we systematically identified 239 groups of sister isolates (defined as lacking in core genome SNP differences and having very low pairwise nucleotide differences, FIG. 8 and data not shown) and identified gene content differences within each group. Strikingly, over half of the gene clusters in the pan-genome (5853 out of 10583) were differentially present between isolates in at least one of the 239 groups of sister isolates (data not shown, hereafter referred to as “differential genes”). We note that most of these differential genes (n=4217) were unlikely identified due to incomplete genome assembly (FIG. 7B, adjusted p<0.001).

Functional heterogeneity between sister isolates ranged from transport and metabolism to cell structure and defense (FIG. 7B) and could result from both gene loss and gene gain events. For example, a differential gene absent in most sister isolates while present in only a small fraction (e.g., K12549, present in 1/11 isolates in the same group, FIG. 7B bottom) more likely resulted from a recent gene gain event. On average, a sister isolate contained 26±29 genes that likely resulted from a gene gain event (i.e., missing in over 50% of the sister isolates of the same group). Alternatively, a differential gene carried by most sister isolates while absent in only a small subset of isolates (e.g., the hemin permease protein K09813, present in 9/11 sister isolates in the same group, FIG. 7B top) more likely resulted from a gene loss event associated with the small subset. Given the large variation in the prevalence of the differential genes among sister isolates (FIG. 7B), both gene gain events and gene loss events likely contributed to the divergence of the sister isolates.

A common mechanism for gene gain events without concomitant accumulation of core-genome SNP diversity is horizontal gene transfer (HGT)—the direct exchange of genetic elements. In the core-genome region, HGT among the subject isolates was likely (suggested by the 104 predicted recombination events, with each isolate affected by 6.2±3.8 events, data not shown) but were of relatively low rate (population-scaled recombination rate=0.14%). For accessory genes, we inferred if HGT contributed to gene content diversity among sister isolates by examining whether the differential genes were observed in mobile element-like contigs. Of the 171 contigs that contained at least 10 differential genes, 53 contigs (with 20 unique contig sequences, FIG. 7C) were predicted as mobile elements using an artificial neural network model implemented in PlasFlow (Krawczyk et al., 2018), again suggesting that HGT is likely. In addition to mobile-element-like contigs, we identified 25 unique chromosome-like contig sequences (FIG. 7C). Interestingly, at least two mobile element-like contigs appeared to have integrated into multiple chromosome-like contigs, as defined by nearly 100% alignment over the length of the contigs (FIG. 7C). These were annotated as phage sequences (FIG. 7D), further indicating that mobile elements such as phages could dynamically drive the divergence of sister isolates recently descended from a common ancestor.

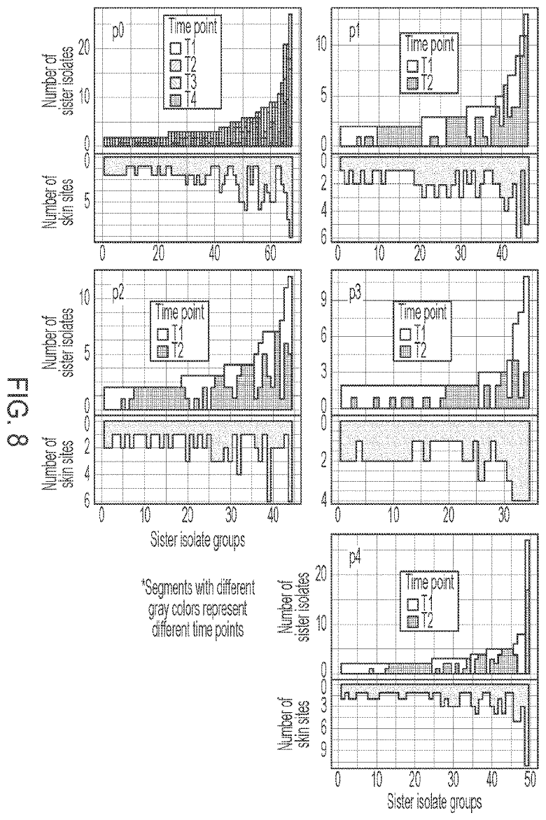

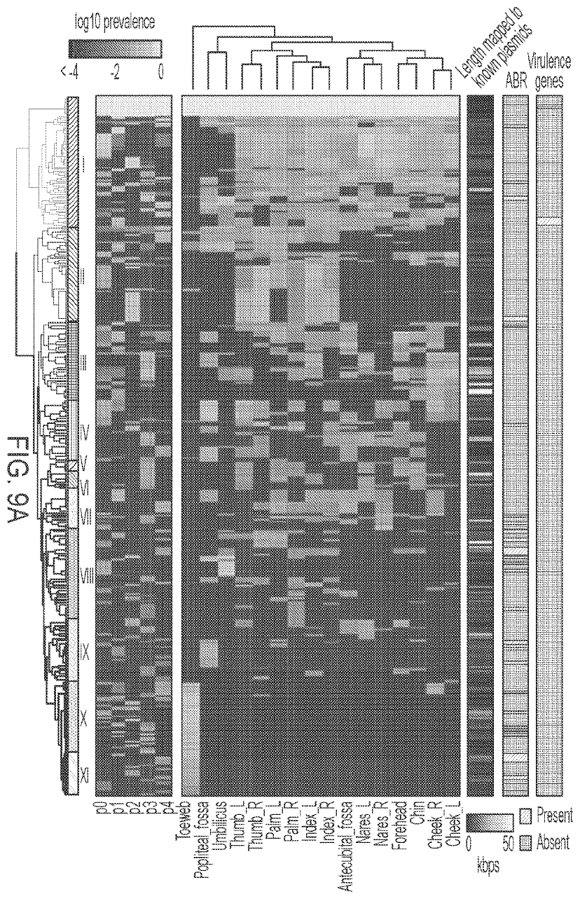

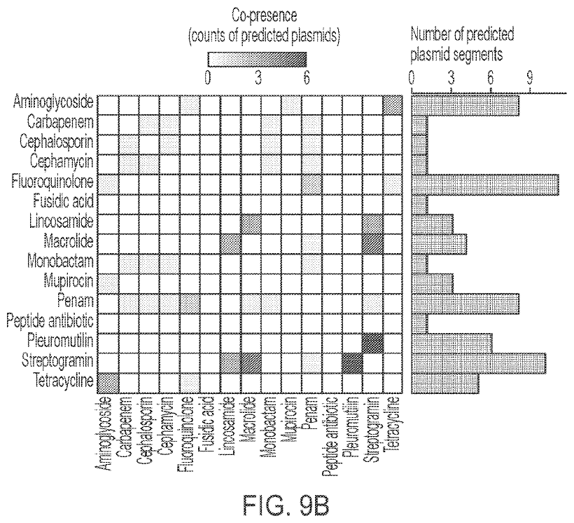

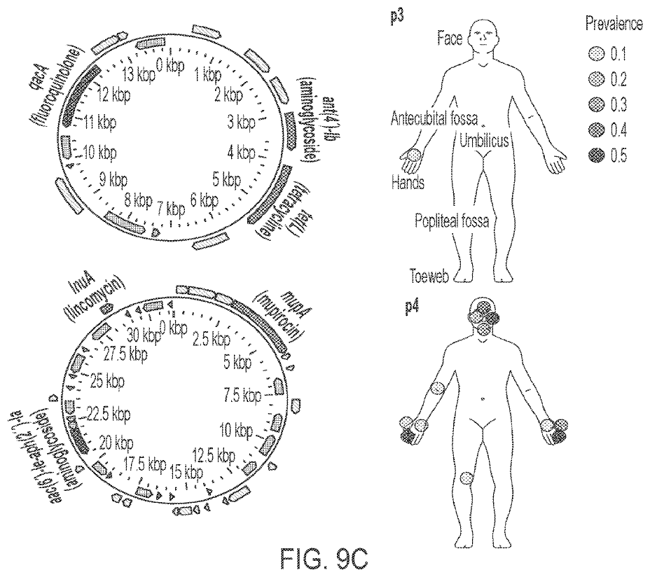

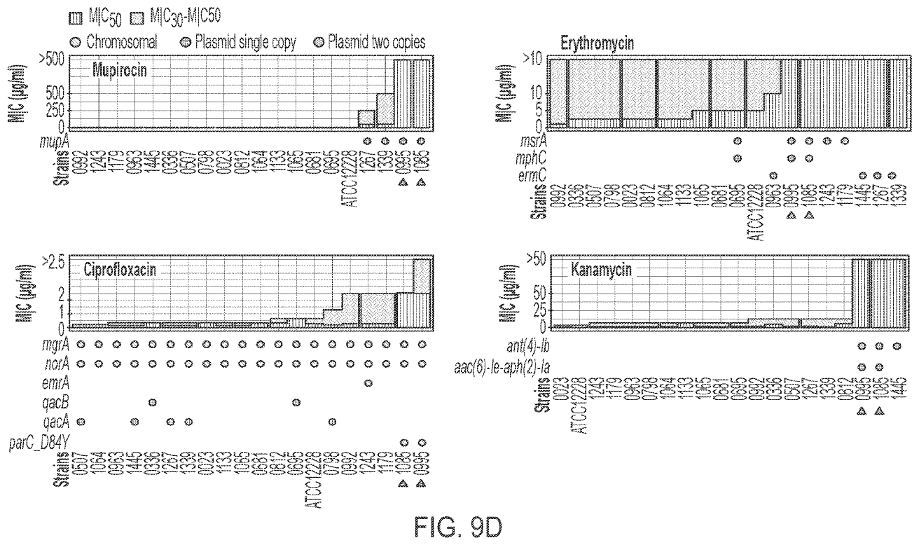

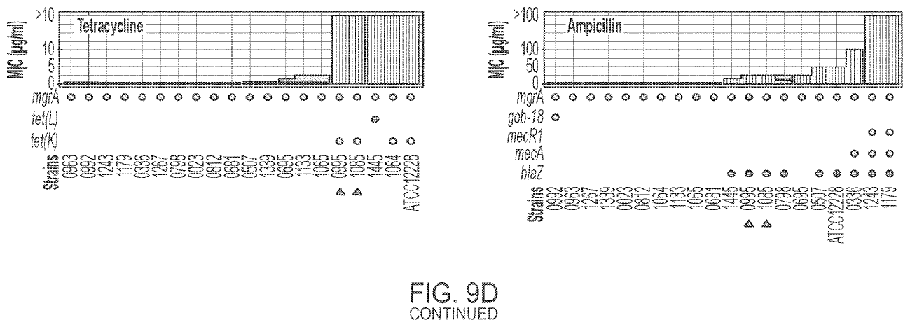

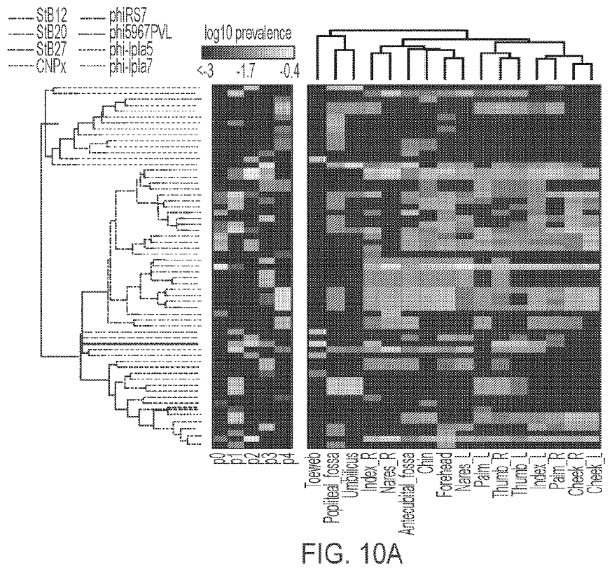

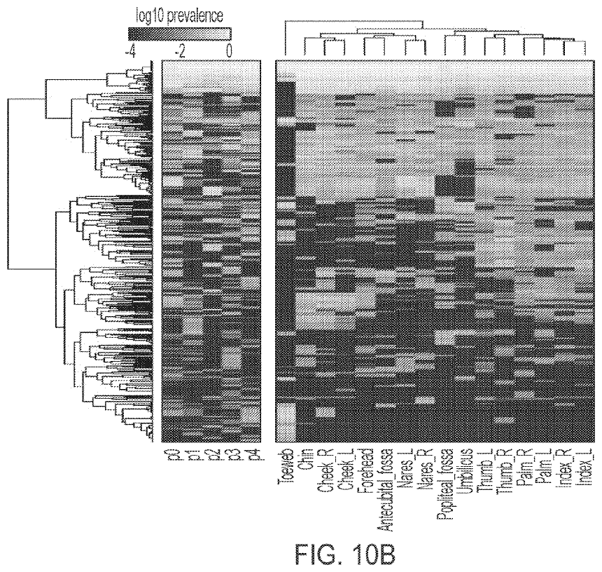

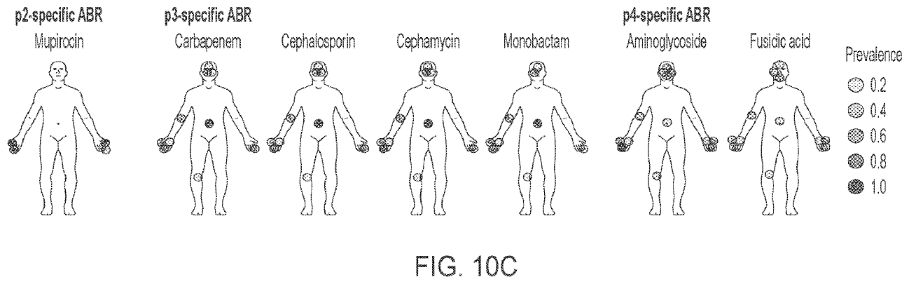

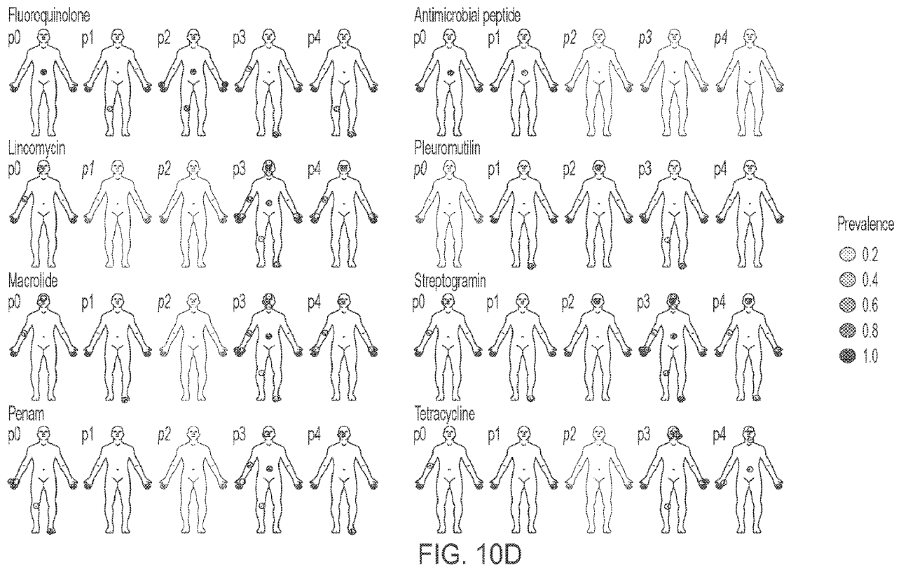

Both predicted plasmid segments (383 unique) and phages (61 unique) had significantly different prevalence across subjects and skin site (FIGS. 9A and 10A, for both, PERMANOVA based on Euclidean distance p<0.001 for both skin site and subject). As our genomes are draft quality, it is unclear which predicted plasmid segments are physically located on the same replicon (mapped to 9.5±1.6 contigs per genome), though clustering based on skin site distribution showed possible physical or functional linkages (FIG. 9A). Two clusters, X and XI, were strongly associated with toeweb isolates (FIG. 9A), suggesting that the toeweb subpopulation contained unique mobile elements. Even when only considering previously identified plasmids (4.6±2.7 predicted plasmid contigs/genome), the toeweb subpopulation still possessed a unique set of predicted plasmid segments (FIG. 10B). Taken altogether, given that plasmids and phages commonly mediate HGT, the observed association between subjects, skin sites and predicted plasmid/phage types suggested that S. epidermidis subpopulations could access different sets of HGT genes at different skin sites and in different hosts.

### Example 3: Functional Consequences of Population-Level Diversity

Given the role of S. epidermidis both as an opportunistic pathogen and a gene reservoir for other skin pathogens such as S. aureus (Archer and Johnston, 1983; Forbes and Schaberg, 1983; Méric et al., 2015), we next examined if the observed genetic diversity of S. epidermidis could have functional consequences that could impact its role in skin health and disease. We particularly examined mobile gene elements, given that they can shape the gene content landscape of S. epidermidis and potentially contribute to the spread of virulence factors and antibiotic resistance genes (ABR).

Importantly, a significant fraction of predicted plasmid segments (FIG. 9A, 39 out of 383) but no predicted phage sequences contained ABR genes, suggesting that their transfer would primarily occur by plasmid. Note that all predicted plasmid-borne ABR genes, except for a fusidic acid inactivation enzyme (fusC), showed homology to genes in known plasmids. Only five predicted plasmid segments carried predicted virulence genes (FIG. 9A). Overall, the distribution of predicted plasmid-encoded ABR was highly host-specific and skin site-specific (FIGS. 10C-10D), further underscoring the biogeographical heterogeneity of S. epidermidis functional features. Predicted plasmid-encoded ABR genes were predicted to confer resistance to at least 15 types of antibiotics (FIG. 9B, data not shown), including mupirocin and streptogramin (FIG. 9B, Table 6), recently developed antibiotics used specifically to treat Staphylococcus skin infections, raising concerns for the long-term effectiveness of these drugs. Moreover, many predicted plasmid segments encoded resistance against multiple antibiotics (FIG. 9B), due to pleiotropy and/or co-presence of ABR genes and mechanisms (FIG. 9B, data not shown). For example, we predicted two multi-drug resistance (MDR) plasmid segments, each with three distinct ABR genes targeting three distinct types of antibiotics and observed in only one subject (FIG. 9C), respectively.

To validate the functionality of predicted ABR genes, we measured the minimum inhibitory concentration (MIC) of six antibiotics suppressing the growth of 19 selected isolates that possessed different ABR genes (FIG. 9D). We found that computational predictions of plasmid-encoded ABR genes were, in general, good predictors of the actual resistance phenotypes (FIG. 9D), while chromosomally predicted ABR genes, such as mgrA and norA, often failed to confer similar levels of resistance as the predicted plasmid-encoded ABR genes. An exception was resistance to ciprofloxacin, a fluoroquinolone antibiotic, which is likely endowed by a previously reported mutation in the DNA topoisomerase ParC (D84Y) (Yamada et al., 2008), but not by the predicted chromosomal or plasmid-encoded efflux pumps (FIG. 9D). In addition, chromosomally predicted mecA and mecR1 genes that encode and regulate a penicillin binding protein appeared to compound the resistance conferred by the predicted plasmid-encoded beta-lactamase BlaZ (FIG. 9D). Finally, isolates 0995 and 1085, harboring an MDR plasmid (FIG. 9C, lower) in addition to other resistance genes, exhibited resistance to all six tested antibiotics (FIG. 9D, arrows). These results demonstrate the individualization of MDR phenotypes and the association with spread of predicted MDR plasmids.

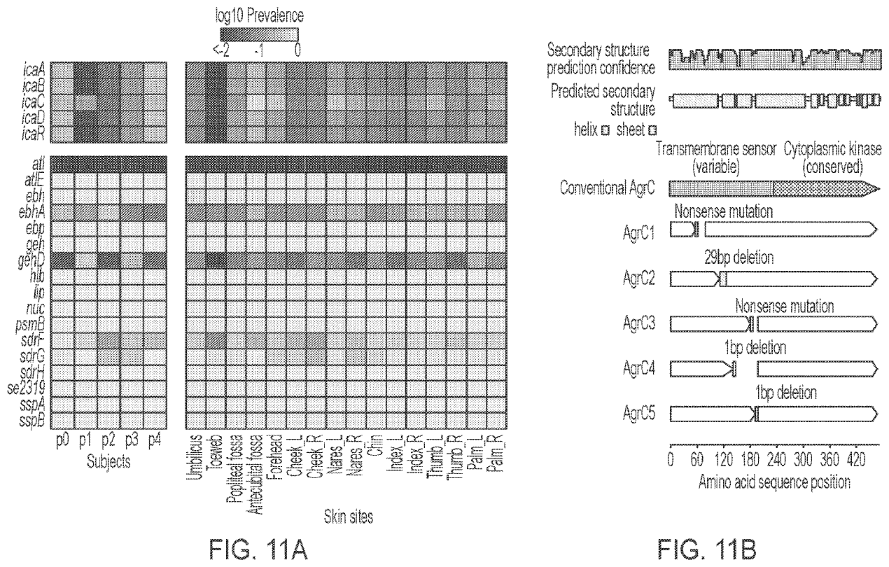

A common assumption about staphylococcal infections or skin disease is that the etiological agent originated from the patient's own skin (von Eiff et al., 2001; Kong et al., 2012; Méric et al., 2018; Otto, 2009; Sakr et al., 2018). Thus, understanding the biogeographic distribution of virulence factors and how they are regulated in their respective microenvironment could aid the assessment of infection risk and guide intervention approaches. Similar to other functional features, predicted S. epidermidis virulence genes showed varying prevalence among subjects and skin sites (FIG. 11A, PERMANOVA based on Euclidean distance, p<0.001 for both skin site and subject), including a complete absence of the ica operon (icaA, icaB, icaC, icaD and icaR genes, important for biofilm formation) in all toeweb isolates and the majority of isolates from p1 (FIG. 11A).

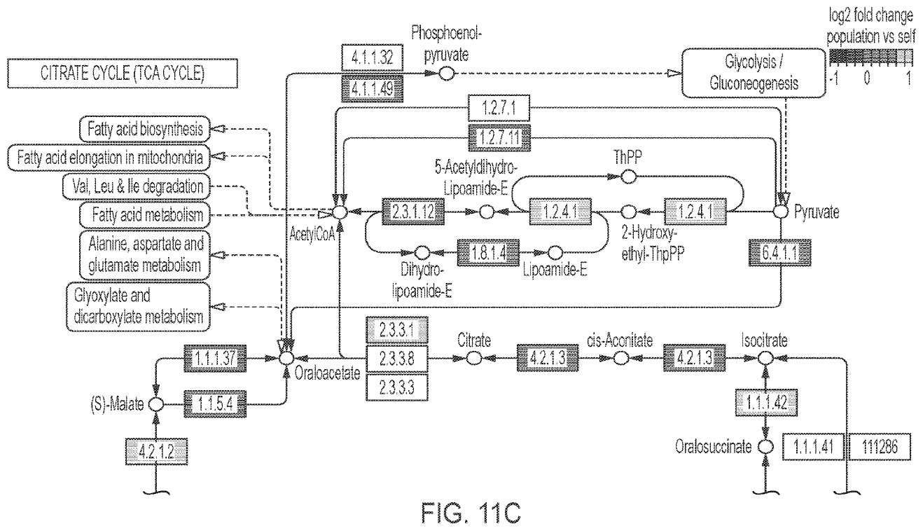

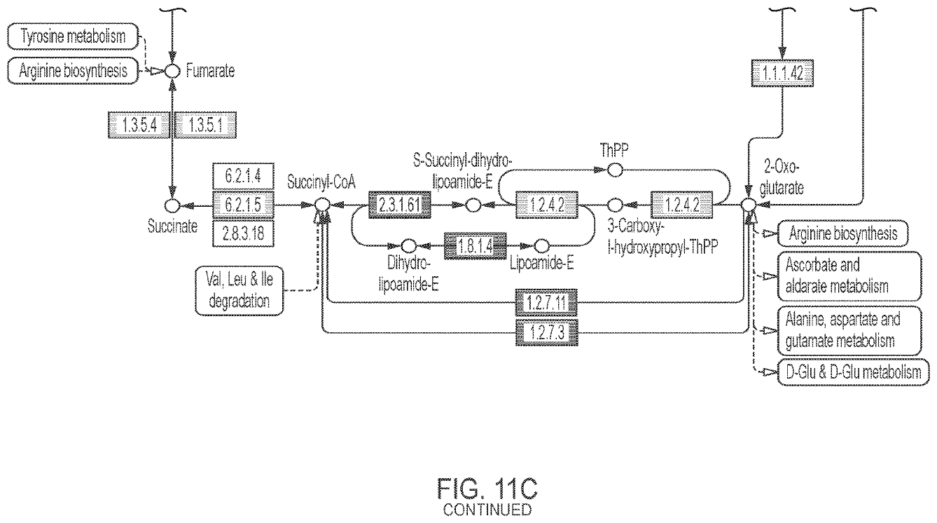

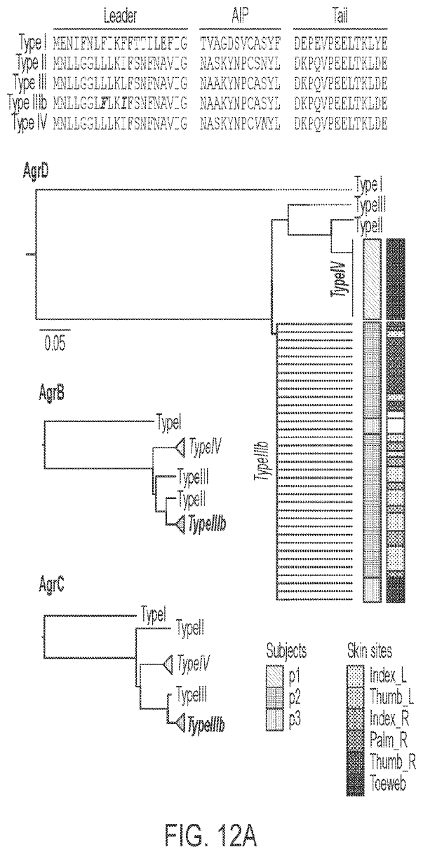

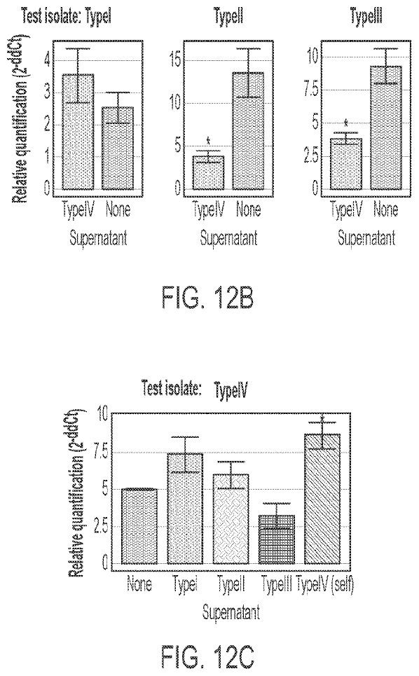

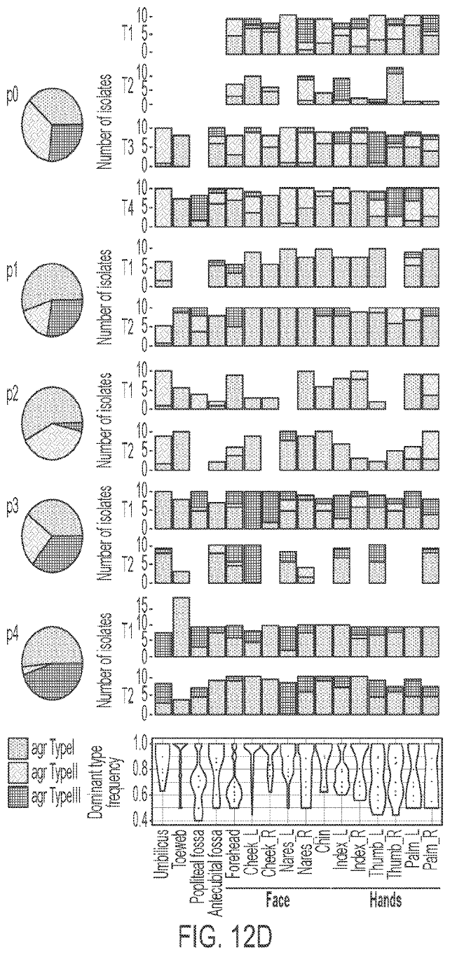

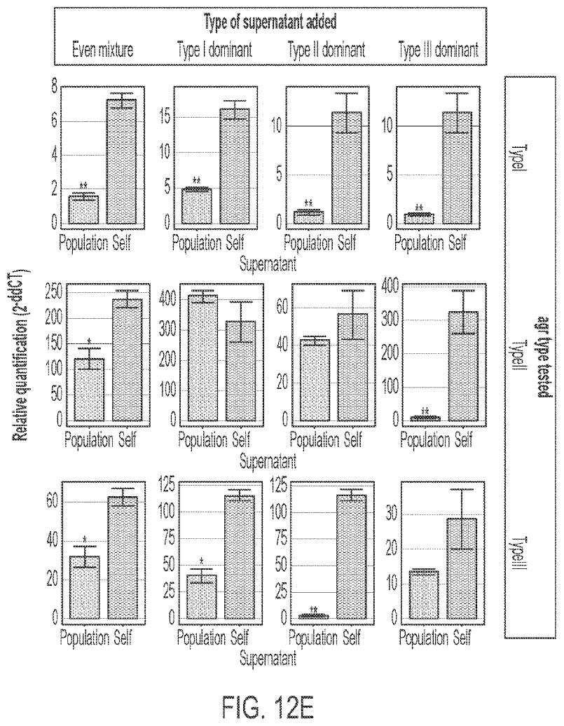

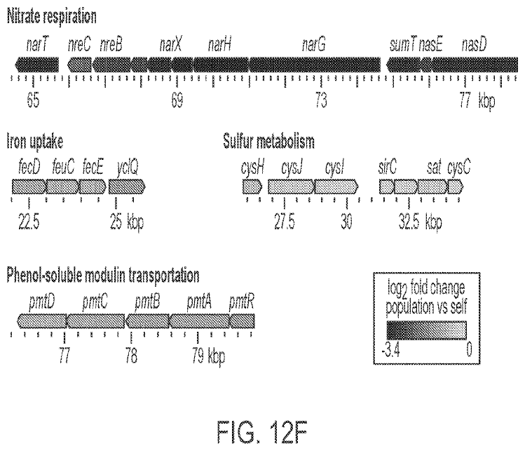

An additional regulation of many staphylococcal virulence factors is enforced by the agr quorum sensing system, encoded by the agrABCD operon (Méric et al., 2018; Yarwood and Schlievert, 2003). agr quorum sensing controls the expression of many extracellular virulence factors important for dissemination during acute infection (Fey and Olson, 2010; Olson et al., 2014), while down-regulated agr activity was associated with colonization and persistence (Le and Otto, 2015). The agr system produces an autoinducing peptide (AIP, encoded by the agrD gene, FIG. 12A) secreted through AgrB, detected by AgrC, which then activates the response regulator AgrA. Previous studies showed considerable sequence polymorphism in the S. epidermidis agr locus (Olson et al., 2014), with three ‘types’ identified based on the sequence of AgrD. Notably, Olson et al. emphasized the importance of these sequence variations in strain-level competition: one type of AIP can inhibit the agr system of a different type. Therefore, we hypothesized that agr type admixture in the skin could suppress virulence depending on the composition of agr types in the subpopulation.

We thus examined agr diversity in the subject isolates. We identified canonical agr sequences as well as agrC transmembrane mutants observed in multiple subjects and skin sites (FIG. 11B) and two novel agr sequence variants, Type IIIb and Type IV, that had highly restricted subject and skin-site distribution (FIG. 12A). While Type IIIb expresses the same AIP as Type III (but with a unique AgrD leader peptide), Type IV expresses a unique AIP, and its supernatant was able to interfere with quorum sensing of Type II and III strains (FIG. 12B and data not shown, Welch's t-test p<0.05), as measured by reduced ecp expression, an agr-regulated protease (Olson et al., 2014). Conversely, when a Type IV isolate was grown with spent media supernatant of Type I-III isolates, no significant difference in ecp expression was observed, potentially due to large variance (FIG. 12C, data not shown).

Surprisingly, we identified a strong bias in agr types across individuals (FIG. 12D), with p2 isolates consisting predominantly of agr Type I and Type II (adjusted p<10-13) and p4 agr Type I and Type III (p<10-20). At the same time, admixture of agr types within subpopulations was very common (FIG. 12D). To examine the functional consequence of different levels of agr admixture, we measured ecp expression levels when an isolate was exposed to a mixed supernatant from isolates reflecting real-world admixtures (‘population supernatants’). As agr types existed in various proportions on human skin (FIG. 12D), population supernatants were correspondingly created with different proportions of agr types. Strikingly, across all agr types, when combined with nearly all population supernatants, ecp expression was significantly reduced compared to the self-supernatant control (FIG. 12E, data not shown), indicating that real-world strain admixture can reduce quorum sensing and potentially population virulence.

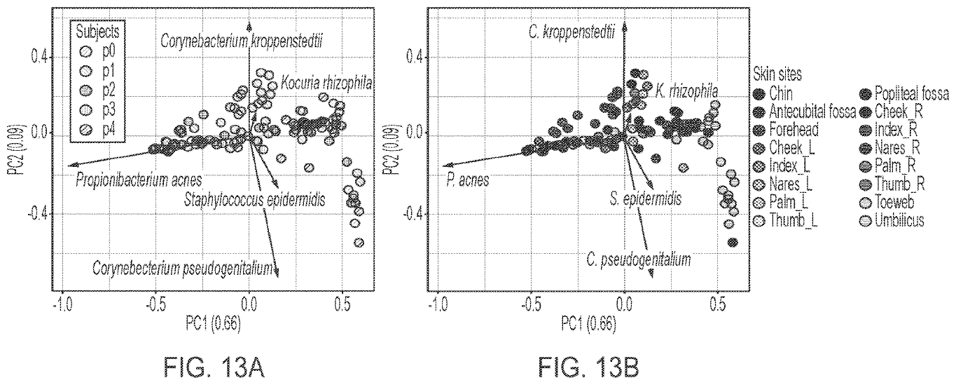

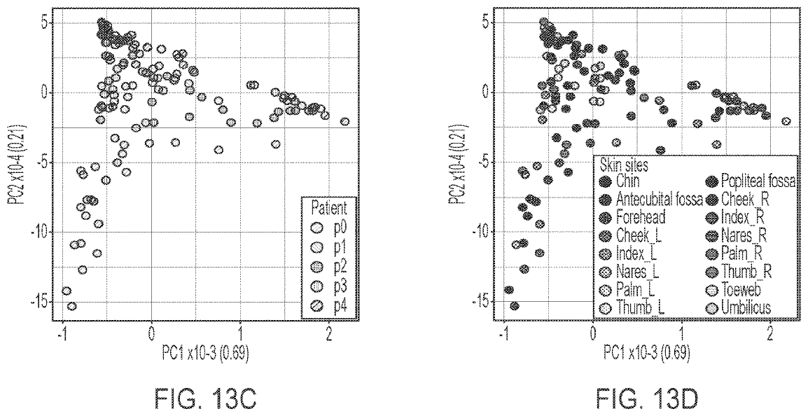

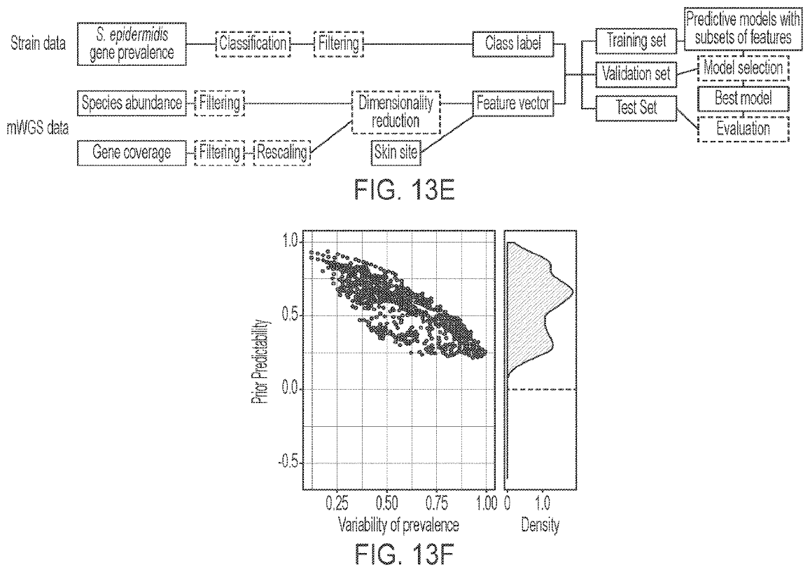

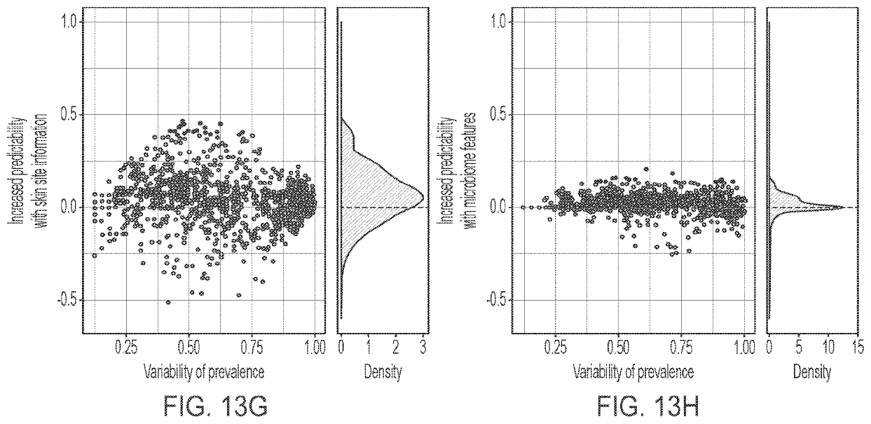

As agr quorum sensing can define the functional state of a population by controlling diverse biological processes from basic metabolism to virulence and pathogenesis, we asked how admixture of agr types in natural populations can affect the functional profile of strains in that population. Exposure of an agr Type I isolate to population supernatant significantly altered expression levels of a variety of operons and pathways, including metabolic gene expression, as measured by RNA-seq (FIGS. 12F and 13C, data not shown). Consistent with previous reports (Batzilla et al., 2006; Queck et al., 2008; Yao et al., 2006), genes involved in nitrogen metabolism and urease activity were downregulated in the presence of population supernatant, while pathways involved in carbohydrate metabolism were upregulated (FIGS. 12F and 13C, data not shown). Contrasting with a previous study (Batzilla et al., 2006), we found that genes involved in sulfur metabolism were down-regulated with population supernatant (FIG. 12F, data not shown), underscoring potential strain-specific effects. Interestingly, we also identified changes in the expression of potential virulence factors in an admixture. For example, expression of the pmt locus was suppressed when exposed to population supernatant (FIG. 12F, data not shown). pmt is responsible for the export of phenol-soluble modulins (PSMs), a major staphyloccal virulence factor (Cheung et al., 2014; Wang et al., 2011). Genes involved in iron uptake—fecD, feuC, fecE, yclQ—were also downregulated in the presence of population supernatant (FIG. 12F, data not shown). While iron acquisition's role in virulence has been extensively studied (Oliveira et al., 2017; Trivier and Courcol, 1996; Trivier et al., 1995), to our knowledge, its association with quorum sensing has not yet been demonstrated in S. epidermidis, although an association with cell density has been discussed (Matinaho et al., 2001). By our experimental simulation of real-world admixture, we demonstrated that reduction of predicted virulence factors by quorum sensing interference is at least one functional consequence of strain heterogeneity and could potentially aid S. epidermidis' survival as a skin commensal or persistence as a pathogen.

### Example 4: S. epidermidis Gene Content is Influenced by the Local Skin Microbiota

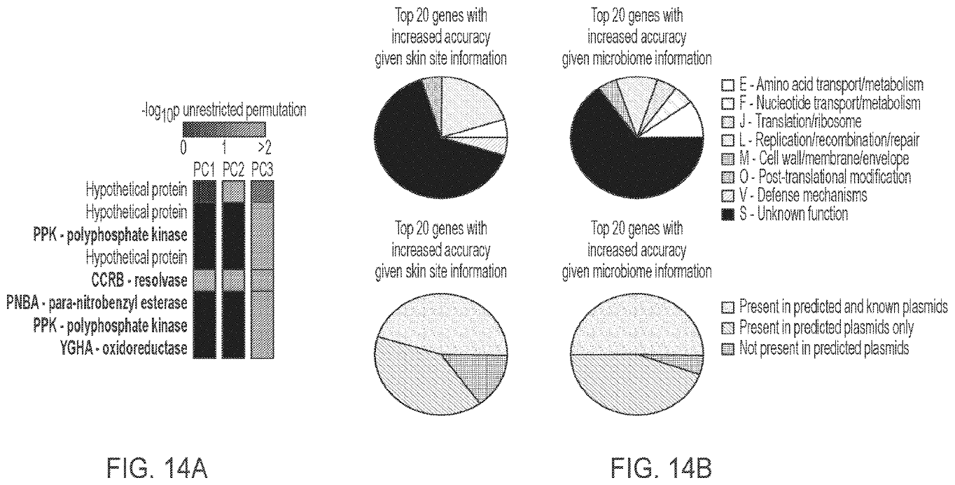

Interspecies interactions with the contextual microbiota may also shape S. epidermidis strain genetic diversity via metabolic potential, resource competition, or antimicrobial activity. We thus analyzed metagenomic shotgun data obtained as matched samples with the isolates. Consistent with our previous reports (Oh et al., 2014, 2016), the healthy skin microbiome is characterized by a remarkable biodiversity across hosts (FIG. 13A, data not shown) and skin sites (FIG. 13B, data not shown). Using a generalized linear model, we found 8 S. epidermidis genes (out of 1130 genes filtered based on abundance, variation, and sparsity) significantly associated with microbial taxonomic composition (FIG. 14A, adjusted p<0.05 for unrestricted permutation and permutation restricted within subject/subject×skin site), which was represented by three principle components that explained 82% of the variation. For example, polyphosphate kinases are important for synthesizing polyphosphate, which is needed for bacterial survival under stress conditions (Zhang et al., 2002). ccrB is involved in the integration and excision of HGT elements (Wang and Archer, 2010), suggesting a linkage between population-level resistance prevalence and the contextual microbiome. pnbA is linked with beta-lactam production in Bacillus (Zock et al., 1994), although its function in staphylococci has not been fully studied. Despite these diverse functional associations to microbiome species abundances, no S. epidermidis gene showed significant association with microbiome gene abundances (FIGS. 13C and 13D), potentially because of low power due to small sample size relative to the large number of S. epidermidis genes tested.

Another fundamental question in human microbiome research is how much of microbiome features at the population level—both genetic diversity and ecological interactions—is host-specific vs. generalizable to different hosts. We created a machine learning model (FIG. 13E) to study skin site and microbiome features (in subjects p0, p1, p2, and p4) that increase S. epidermidis gene predictability in a new host (subject p3). Prevalence of many genes in the new host can be predicted without any site or microbiome information (FIG. 13F, genes with high “prior” predictability), representing genes with similar prevalence (i.e., low variability of prevalence in FIG. 13F) at all skin sites in all subjects (e.g., core genes encoding universal functions).

Other genes showed increased predictability when including skin site and microbiome information (FIGS. 13G and 13H), potentially indicating a role in environmental specialization or interspecies dynamics. The biological functions of the top 20 such genes were largely unknown and showed limited consistency (FIG. 14B, upper), but most were present in predicted plasmid segments (FIG. 14B, lower). Additionally, over half (52.9% and 52.6%) of these predicted plasmid-associated genes also have homologs observed in previously identified plasmids (FIG. 14B, lower). An important conclusion from this analysis is thus that features associated with skin niches and the surrounding microbiome consistently influence S. epidermidis mobile elements, and therefore HGT is contingent on the state of the contextual environment.

A key finding of our study is the extensive within-individual variation of S. epidermidis at the population level. While our approach can be generalized to understand population diversities of other human-associated bacteria, we believe that biological dynamics will be individual, body-site, and microbe-specific and must be interrogated as such. For example, the within-individual evolution of S. epidermidis differs substantially from a gut microbe, B. fragilis. The within-host S. epidermidis isolates maintained the genetic variation of multiple colonizing lineages, while within-host B. fragilis only represented a single colonizing lineage (Zhao et al., 2019). This suggests that a diverse pool of S. epidermidis founder strains is maintained in the environment and subsequently colonizes healthy individuals. Two fundamental questions that follow are: 1) how is the polymorphism of founder lineages maintained in the environment? and 2) how is a diverse set of founder strains transmitted to each individual? We speculate that both questions could be explained by the fact that the major reservoir of S. epidermidis is the mammalian skin, which includes various environmental niches within one individual. Multiple-niche polymorphism is known to maintain diversity in natural populations (Brisson, 2018; Brisson and Dykhuizen, 2004; Dobzhansky, 1982), and could increase the exposure of a recipient to multiple founder strains simultaneously. On the other hand, it is also possible that different lineages of B. fragilis are maintained in an individual, but are not identified because bacteria occupying certain gut niches are known to be underrepresented in fecal samples (Zmora et al., 2018).

The prevalence of S. epidermidis gene content and other genetic features exhibited marked skin site-specificity, suggesting functional specialization to the niche. For example, the unique population structuring of toeweb isolates, which possessed distinct gene contents and functional features irrespective of host, could be due to both a lack of gene flow because of low transmission rates, and niche adaptation. Although the number of subjects in our study is limited and therefore the generality is unclear, the observed convergence suggested purifying selection at the toeweb, and adaption of the toeweb subpopulation to the toeweb niche. If the purifying selection at the toeweb is rapid and strong enough, a distinct subpopulation could be formed even without ecological isolation, albeit this hypothesis is less parsimonious than ecological isolation. An interesting corollary to these findings is the ecology of other toeweb microbes, which could face similar evolutionary pressures. For example, based on metagenomic inference, Cutibacterium acnes may also have phylogenetically distinct strains that are associated with the toeweb (Oh et al., 2014). On the other hand, clinically important features of S. epidermidis, including ABR and predicted virulence genes, were strongly host-specific and showed dynamic HGT between skin sites, including predicted ABR-encoding plasmids. This provides strong support for pathogen carriage and increased infection risk elsewhere in the body, such as of methicillin-resistant S. aureus in the nares (von Eiff et al., 2001; Kong et al., 2012; Sakr et al., 2018), as well as for the contextual microbiome affecting infection risk via HGT of pathogenicity reservoirs.

In addition to ABR, we found that the distribution of agr types, including two novel types, was highly host-dependent. A key observation was the substantial admixture of multiple agr types, which significantly repressed quorum sensing in vitro and consequently altered expression of a variety of biological functions from metabolic control to virulence. Indeed, clinically, admixture of agr types might represent a mechanism by which virulence could be suppressed at the population level (vs. gene-level mechanisms such as the absence of predicted virulence factors, which may be the case in foot isolates, or physical factors such as low cell density, which may contribute at other skin sites). If population bottlenecks occur such that a single agr type becomes dominant in a subpopulation, increased expression of virulence genes could then facilitate acute infection. This hypothesis would also account for the inability of genomic studies on S. epidermidis to date (Conlan et al., 2012; Méric et al., 2018) to identify clear determinants of pathogenicity among nosocomial and commensal isolates.

Recent developments in metagenomic analyses have used SNP-based haplotypes to infer strains from shotgun metagenomic data, either by reconstructing the dominant strain haplotype (Truong et al., 2017), or by phasing SNPs under a probabilistic model (O'Brien et al., 2014; Quince et al., 2017; Smillie et al., 2018). Despite their reported applications to resolving individual-specific strain diversity (Segata, 2018; Tett et al., 2017; Zhang and Zhao, 2016) and tracking strain transmission between individuals (Asnicar et al., 2017; Brito and Alm, 2016; Ferretti et al., 2018; Smillie et al., 2018; Yassour et al., 2018), these methods are limited by sequencing depth (which makes them unsuitable for low abundance species), restricted to a few marker gene regions (which decreases phylogenetic resolution), and insensitive to haplotypes with few SNP differences (and thus limited in the ability to resolve closely related genomes). This latter is particularly limiting as differentiating sister isolates with 0 SNP differences would be impossible. Nonetheless, we found that such sister isolates were exceptionally informative. This is because sister isolates likely have diverged very recently from the same parental lineage. Thus, sister isolates detected at different skin sites likely denote transmission events, while sister isolates with different gene content likely denote divergence by either gene loss or HGT. Indeed, a comparison of the identified sister isolates not only revealed recent transmission events, but also differential gene content with a range of biological functions between sister isolates. Additionally, we found that many of the differential genes were clustered on predicted mobile-element-like elements, suggesting that HGT dynamically contributes to the divergence of sister isolates. Put together, the ability to resolve sister isolates for functional and demographic inferences represents an important advantage of isolate WGS over metagenomic-based approaches.

On the other hand, metagenomic sequencing characterizes the microbial macroenvironment of the S. epidermidis isolates and can shed light on how environmental selection influenced their evolution. We identified multiple accessory genes of S. epidermidis whose prevalence was significantly associated with features of the contextual microbiome. In addition, we also found that some of the ecological interactions between S. epidermidis genes, especially mobile genes, and the contextual microbiome could be generalized to new hosts. This finding suggested that it may be possible to infer strain-level functional differences, including infection predilection, based on skin microbiome features of the host. We note that this possibility could be valuable to many other microbes where large scale culturomics are challenging due to the lack of well-defined, selective culturing conditions or screening/characterization methods.

Nonetheless, we note several limitations of these inferences, and of an approach such as ours more generally. First, due to sequencing depth and technical limitations in pooled metagenomic assembly, a reconstructed metagenomic gene catalog will not fully reflect coding potential of the contextual microbiome. Second, although within-host diversity was well-captured by our dataset, such randomly sampled isolates are inevitably an incomplete representation of the corresponding subpopulations and the small number of recruited subjects, hindering the detection of sister isolates and decreasing the confidence in estimated transmission probabilities. This may be particularly relevant for S. epidermidis, whose subpopulations may frequently experience dynamic perturbations in the skin, resulting in population bottlenecks and consequently, genetic drift (all the while on the community scale, skin microbiome composition is relatively stable (Oh et al., 2016)). The presence of population bottlenecks is suggested by the lack of bilateral symmetry in both the transmission patterns, gene content diversity, and the temporal fluctuations patterns. A deeper sampling of each skin site and a denser time series will likely improve characterization of such demographic dynamics. Additionally, while we believe that the underlying evolutionary mechanisms that shaped the population diversity will be generalizable to other subjects and even other skin microbes, the generality of the ecological and functional interactions found in these subjects could be limited given the large degree of host-specificity. Third, the ability to make meaningful functional inferences from our findings will require a more comprehensive characterization of gene functions (although here, computational predictions of plasmid-encoded antibiotic resistance genes were largely consistent with experimental validation).

### Example 5: Materials and Methods

Five (5) healthy males and females aged between 20-60 years were recruited to this study, which was approved by the Jackson Laboratory Institutional Review Board. Due to the limited sample size, we do not report specific ages or genders to protect confidentiality. Exclusion criteria included any visible signs of non-intact skin at sites of sampling, use of systemic antibiotics or antifungals within 3 months prior to enrollment, or topical retinoids, steroids, or antibiotics within 1 week prior to enrollment. Sixteen skin sites (data not shown) were swabbed rigorously using PurFlock Ultra buccal swabs (Puritan Medical Products) for thirty seconds before the swab was submerged into 500 μl Tryptic Soy Broth (TSB) culture media (Thermo Fisher Scientific). The same swab was used for both the isolation of S. epidermidis and mWGS sequencing. For the isolation of S. epidermidis, 50-100 μl of the culture were plated onto SaSelect culture plates (Bio-Rad Laboratories) and incubated at 37° C. for 24 hours. Small and light pink colonies (Hirvonen et al., 2014) were picked randomly from the plates and verified on the MALDI Biotyper system (Bruker Corporation) according to the manufacturer's instructions. In general, from each skin swab, we aimed at obtaining ˜10 colonies annotated as “S. epidermidis” by the Biotyper, which were subsequently inoculated into 1.5 mL TSB and grown overnight for DNA extraction.

### DNA Extraction

Rapid DNA extraction from S. epidermidis isolates were adapted from Köser et al. (2014). 1 mL of overnight culture was centrifuged at 20,000×g for 1 minute before the bacterial pellet was resuspended with 100 μL of 1×TE and transferred to a 2 mL bead beating tube with 100-125 μL 0.5 mm diameter glass beads (BioSpec Products). An additional 100 μL of 1×TE was added to the tube, followed by vortexing of the sample for 30 seconds at max speed (3000 rpm) on a Vortex Adapter (Mo Bio Laboratories). The mixture was then centrifuged at 13,000×g for 5 minutes to pellet the cellular debris, and the supernatant was transferred to a new tube to be used as template for Nextera XT library preparation.

For the extraction of metagenomic DNA, a skin swab was placed into a microfuge tube containing 350 μL Tissue and Cell lysis buffer (Epicentre) and 100 μg 0.1 mm zirconia beads (BioSpec Products). Metagenomic DNA was extracted using the GenElute Bacterial DNA Isolation kit (MilliporeSigma) with the following modifications: each sample was digested with 50 μg of lysozyme, and 5 units lysostaphin and mutanolysin for 30 minutes prior to beadbeating in the TissueLyser II (QIAGEN) for 2×3 minutes at 30 Hz. Each sample was centrifuged for 1 minute at 15000×g prior to loading onto the GenElute column. Negative (environmental) controls and positive (mock community) controls were extracted and sequenced with each extraction and library preparation batch to ensure sample integrity.

### Library Preparation and Sequencing

All sequencing libraries were made according to the Illumina standardized protocol using the Nextera XT DNA sample preparation kit (Illumina Inc.). All DNA samples were quantitated by Qubit HS (Thermofisher Scientific) and diluted to 1 ng/μl. The dual indexed paired-end libraries of genomic DNA were made with an average insert size of 400 bp by taking 200 μg DNA of each sample in optimized quarter reaction protocol, where all reagents for library preparation were taken in ¼th amount. Tagmentation and PCR reactions were carried out according to the manufacturer's instructions. The resulting Nextera WGS libraries were then sequenced with 2-150 bp paired end reads on an Illumina HiSeq2500.

### Genome Assembly and Quality Filtering

Sequencing adapters and low-quality bases were removed from the sequencing reads using scythe (v0.994) (Buffalo) and sickle (v1.33) (Joshi and Fass), respectively, with default parameters. Filtered sequencing reads were then assembled using SPAdes (v3.7.1) (Bankevich et al., 2012), with default parameters. We took a series of strategies to remove low quality samples or contigs. First, draft genomes with sizes smaller than 2.2 Mbps or larger than 2.9 Mbps—sizes unlikely for S. epidermidis (genome sizes of 50 public S. epidermidis genomes in the VCU and NIH collection ranged from 2.3-2.8 Mbps [data not shown])—were removed from the dataset. Second, qualities of the remaining draft genomes were checked using QUAST (v4.2) (Gurevich et al., 2013) by aligning to a S. epidermidis reference genome (strain ATCC12228, ACC #: GCA_000007645); draft genomes with a reference coverage of lower than 85% were removed from the dataset (“genome fraction” in the QUAST output; reference coverages of 50 public S. epidermidis were all higher than 86%). Third, contigs with lower than 10× coverage, which contained potential contaminations based on taxonomic classification using Kraken (v0.10.6) (Wood and Salzberg, 2014) (Figure S1), were removed from the draft genomes. Finally, sample purity was checked by mapping sequencing reads back to the draft genomes using Bowtie2 (v2.3.1) (Langmead and Salzberg, 2012; Langmead et al.), and calling SNPs at sites with at least 10×coverage using bcftools (v1.8) and samtools (v1.8, vcfutils) (Li, 2011; Li et al., 2009). Samples with >10 sites having>0.5 allele frequencies of the variant alleles, which strongly indicated an admixture of S. epidermidis isolates, were removed from the dataset.

To validate the sequencing and quality filtering methods, S. epidermidis strain ATCC12228 was sequenced, assembled and quality filtered as described above, and aligned to its published complete genome sequence (GCA_000007645) using QUAST (v4.2) (Gurevich et al., 2013). 99.2% of the bases in the resulting draft genome (2.54 Mbps in 95 contigs, N50=71, 627) were aligned to the complete reference genome, representing 98.0% of the reference genome (“genome fraction” in the QUAST output), with a mismatch ratio of 0.01%. Out of the five contigs (18,932 bps in total) unaligned to the reference genome, four (17,931 bps in total) were mapped to plasmids in S. epidermidis strains PM221 and FDAARGOS_153 (84% alignment coverage and 96% nucleotide sequence identity with BLASTN (Altschul et al., 1990), while the other contig (1001 bps) was mapped to Staphylococcus felis (98% alignment coverage and 81% nucleotide sequence identity with BLASTN). The unaligned regions could represent plasmid discrepancies in our ATCC12228 strain stock and the stock sequenced to generate the complete genome, which was observed between two complete sequences of ATCC 12228 (MacLea and Trachtenberg, 2017; Zhang et al., 2003).

### Time Calibration of Phylogenetic Tree and Inference of Transmission Events

Within-host evolutionary histories—including the time-calibrated phylogenetic trees and transmission events of all isolates from each of the five subjects—were inferred using BEAST (v1.8.4) (Suchard et al., 2018) based on the core-genome alignment constructed using Parsnp (v1.2) (Treangen et al., 2014). Although the evolutionary dynamics of S. epidermidis has not been studied extensively, its close relative, Staphylococcus aureus, does exhibit a clock-like evolution (as an example, see Frisch et al., 2018). Therefore, we assumed that core-genome nucleotides of individualized S. epidermidis isolates change under a strict clock model implemented in BEAST, with nucleotide substitution rate modeled using the Generalized time reversible (GTR) model and uniform mutation rates across branches. As a validation, the mutation rates (nucleotide changes per Mbps per year) inferred based on the model (3.47±1.21 for p0, 1.47±0.68 for p1, 2.27±0.54 for p2, 0.62±0.48 for p3, and 1.42±0.52 for p4) were on the same scale with the estimated mutation rate of Staphylococcus aureus (Duchene et al., 2016) (the mutation rate of S. epidermidis has not been estimated).

Previous studies have demonstrated that high rates of genome recombination (e.g. with a population-scaled recombination rate close to 1%) can influence demographic inference, while correction by removing homoplastic sites may even exacerbate the inference (Hedge and Wilson, 2014). Therefore, we estimated the population-scaled recombination rate using ClonalFrameML (v1.11) (Didelot and Wilson, 2015) with default parameters and a maximum-likelihood starting phylogeny constructed using RAxML (v8.2.12) (Stamatakis, 2014), under the GTRCAT mode. The estimated population-scaled recombination rate was low (0.14%), therefore we proceeded to infer demographic parameters based on whole genome population genetics.

A coalescent tree prior was used with population sizes estimated based on a flexible Bayesian skyline plot (Drummond et al., 2005) with 10 windows. The prior probabilities of the population sizes in each window were assumed to be uniformly distributed between 0 and 10¹⁰⁰. Transmission between sites were estimated using a Bayesian discrete phylogeographic approach (Lemey et al., 2009) with symmetric transmission rates between each pair of skin sites. The approach reconstructed the skin site classification of ancestral nodes in the phylogeny using a standard continuous-time Markov chain, and “transmission” was consequently defined as the change in skin site classification along the phylogeny. Bayesian stochastic search variable selection (BSSVS) procedure was applied to limit the transmission rate parameters to only those that adequately explain the transmission process (Lemey et al., 2009). Finally, BEAST simultaneously infers all of the above evolutionary parameters using Markov Chain Monte Carlo (MCMC). To visualize transmission on a phylogeny, the maximum clade credibility tree was reconstructed using Treeannotator (v1.8.4) (Suchard et al., 2018) included in the BEAST package. Skin site classifications, along with the posterior probability, were mapped onto the phylogeny, and the final phylogeny was visualized as a cladogram using FigTree (v1.4.3) (Rambaut). To summarize the BSSVS results using a transmission map, the log file of BSSVS was analyzed using SpreaD3 (v0.9.6) (Bielejec et al., 2016). Transmission routes with posterior probabilities lower than 0.3 were removed, and the resulting transmission map was visualized using the D3 renderer in SpreaD3.

To assess the consistency of the transmission inference and validate the convergence of MCMC, we first ran two independent MCMC chains based on strains isolated from p0 (n=460) for 500,000,000 and 50,000,000 generations, respectively. Each MCMC chain was sampled every 25,000 generations, with the first 800 samples removed as burn-in. As the transmission maps inferred by the two chains were highly consistent (Figure S2D), strain transmission of p1-p4 was inferred based only on MCMC chains run for 50,000,000 generations.

### Pan-Genome and Core-Genome Identification

Gene coding sequences were predicted from the isolate genomes using Prokka (v1.11) (Seemann, 2014) with kingdom=Bacteria and genus=Staphylococcus. The pan- and core-genomes were identified from the predicted gene coding sequences using the Roary pipeline (v3.11.2) (Page et al., 2015) at 80% identity threshold. The pan-genome and core-genome accumulation curves were computed with 10 iterations. A dendrogram demonstrating the clustering of isolate gene content (FIG. 4C) was plotted using Figtree (v1.4.3) (Rambaut) based on the accessory gene presence-absence matrix (the accessory_binary_genes.fa.newick output from Roary). The core-genome alignment and SNP-based approximately-maximum-likelihood phylogeny were constructed using Parsnp (v1.2) (Treangen et al., 2014) with the reference genome randomly picked from the dataset (parameter -r !).

### Prediction of Recombination

Population-scaled recombination rate was estimated using ClonalFrameML (v1.11) (Didelot and Wilson, 2015) as described above. RDP4 (beta 4.97) (Martin et al., 2015) was used to analyze the genome-wide recombination patterns. 50 representative subject isolates were selected by dividing the full phylogenetic tree into 50 clusters (mean cophenetic distance within each cluster=0.004±0.008, generated using the cutree function in R) and randomly selecting one isolate from each cluster (the selected isolates were specified in Table S1). Core-genome alignment was then constructed for the 50 representative subject isolates using Parsnp (v1.2) (Treangen et al., 2014) as described above. The alignment was then processed using RDP4 using six different algorithms (RDP (Martin and Rybicki, 2000), GENECONV (Padidam et al., 1999), Bootscan (Martin et al., 2005), Maxchi (Smith, 1992), Chimaera (Posada and Crandall, 2001), and 3Seq (Lam et al., 2018)) with default parameters to identify recombination events. Finally, recombination events that were identified by at least two methods were reported. The analyses were conducted with 50 representative isolates instead of the full dataset because 1) similar genome sequences (such as those within each of the 50 clusters) will have relatively low power in recombination detection, and 2) RDP4 compares all triplet combinations within a dataset to detect recombination signals and therefore takes polynomial time with respect to the number of genome sequences.

Significance of differential genes among sister isolates Pairwise nucleotide differences between sister isolates were computed between sister isolates using MUMMER (DNAdiff, v1.3) (Kurtz et al., 2004). Differential genes were defined as gene clusters identified using the Roary pipeline (v3.11.2) (Page et al., 2015) that were only present in a subset of sister isolates but absent in the others. The significance (p-value) of a differential gene—the likelihood that the gene cluster was not found in a subset of sister isolates solely due to genome incompleteness—equals the joint probability that every sister isolate in that subset was incomplete. The incompleteness of the isolate genomes was estimated based on the presence or absence of lineage-specific marker genes using the default lineage_wf work flow in CheckM (v1.0.12) (Parks et al., 2015). The resulting p-values were adjusted following the Benjamini-Hochberg procedure.

### Prediction and Analysis of Phages and Plasmids

Phage sequences were identified from the draft genomes using PHASTER (Arndt et al., 2016). Sequences annotated as “intact” phages were then clustered at 0.9 DNA sequence similarity using CD-HIT (local alignment with alignment coverage threshold of the shorter sequence=0.9) (Fu et al., 2012; Li and Godzik, 2006) to remove highly similar sequences. A dendrogram of the predicted phage sequences was generated based on the accessory gene presence-absence matrix (the accessory_binary_genes.fa.newick output from Roary), as the predicted phage sequences lacked colinear regions.

Plasmid candidates were predicted and filtered using multiple criterions. First, mobile-element-like contigs were identified from all contigs (>1 kb) in the 1482 draft genomes using PlasFlow (v1.1) (Krawczyk et al., 2018)—an artificial neural network-based plasmid prediction approach using sequence base compositions as features. Mobile-element-like contigs were then clustered at 0.9 DNA sequence similarity using CD-HIT (local alignment with alignment coverage threshold of the shorter sequence=0.9) (Fu et al., 2012; Li and Godzik, 2006) to remove highly similar contigs. For increased confidence, we focused on contigs with at least 5 kb of length in this study. The predicted plasmid segments were then screened to remove potential chimeric sequences: first, sequencing reads of the 1482 S. epidermidis isolates were mapped to the candidate plasmid segments using Bowtie2 (v2.3.1) (Langmead and Salzberg, 2012; Langmead et al.) and the coverage of each candidate was computed using Samtools (v1.8, used for samfile to bamfile conversion and sorting) (Li et al., 2009) and Bedtools (v2.27.0, genomecov function) (Quinlan and Hall, 2010). A non-chimeric plasmid segment would likely have either close to 0% or close to 100% of its sequence covered in a S. epidermidis isolate, depending on whether the segment is present or absent in that isolate. For simplicity, candidate plasmid segments with breadths of coverage greater than 80% or lower than 20% in over 90% of the isolates were selected for downstream analyses.

Similarity of the predicted plasmid segments to known plasmids was estimated by first aligning the predicted plasmid segments to the PLSDB plasmid database (release 2018_12_05) (Galata et al., 2019) using dc-megablast (blastn 2.6.0+) (Altschul et al., 1990; Zhang et al., 2000), and then computing the total alignment length to the best-hit plasmid in PLSDB. Clusters of predicted plasmid segments were detected by first hierarchically clustering (the “hclust” function in R by euclidean distance) the predicted plasmid segments based on their prevalence across subpopulations, and then pruning the resulting dendrograms using the “cutreeDynamicTree” function in R package dynamicTreeCut (v1.63.1) (Langfelder et al., 2008) (using the “hybrid” method and deepSplit=4).

Alternatively, to identify contigs that represent segments of known plasmids, we aligned all S. epidermidis contigs to the PLSDB plasmid database (release 2018_12_05) (Galata et al., 2019) using dc-megablast (blastn 2.6.0+) (Altschul et al., 1990; Zhang et al., 2000) and identified contigs that had an alignment coverage greater than 75%. These contigs were then clustered at 0.9 DNA sequence similarity using CD-HIT (local alignment with alignment coverage threshold of the shorter sequence=0.9) (Fu et al., 2012; Li and Godzik, 2006) to remove highly similar contigs. Next, sequencing reads of the 1482 S. epidermidis isolates were mapped to the contigs using Bowtie2 (v2.3.1) (Langmead and Salzberg, 2012; Langmead et al.) and the coverage of each contig was computed using Samtools (v1.8, used for samfile to bamfile conversion and sorting) (Li et al., 2009) and Bedtools (v2.27.0, genomecov function) (Quinlan and Hall, 2010). A predicted plasmids contig with a breadth of coverage over 80% in an isolate were considered present in that isolate.

### Annotation of COG Categories and KEGG Modules

COG functional categories were annotated using the eggNOG-mapper (v4.5.1) (Huerta-Cepas et al., 2016) with default options to prioritize sensitivity. Additional analyses of the unannotated toeweb genes were conducted by searching against the Pfam database (El-Gebali et al., 2019) using HMMER web server (Potter et al., 2018), and conducting enzyme EC number prediction using ECPred (Dalkiran et al., 2018). To annotate KEGG modules and compute module representation, gene sequences were first aligned to a downloaded prokaryotic KEGG gene database (release 2015-08-31) (UBLAST (Edgar, 2010) with an e-value threshold of 10-9 and sequence identity cut-off of 0.5). Next, KEGG ortholog numbers (KO numbers) were assigned to the gene sequences using the ko_genes.list mapping file included in the downloaded KEGG gene database. Finally, the representation of KEGG modules was given by the proportion of KOs in each KEGG module that were found in a given genome, based on the ko_module.list mapping file. The KOs that had differential prevalence among subjects or skin sites were identified using ANOVA, with p values estimated using unrestricted permutation and adjusted under the Benjamini-Hochberg procedure.

### Annotation of Virulence Factors and ABR Genes

Known virulence factors were annotated by blasting gene sequences against the Staphylococcus-specific genes in VFDB (Chen et al., 2016), with the addition of four phenol-soluble modules (sequences based on Otto et al. (2004)), using UBLAST (USEARCH v8.0.1517) (Edgar, 2010) with an expect value (e-value) threshold of 10-9. ABR genes were annotated using the Resistance Gene Identifier (RGI, v4.2.2) based on the CARD database (v3.0.1) (Jia et al., 2017), with the low_quality mode and plasmid data-type. Presence of homologs of ABR genes in known plasmids were estimated by aligning the genes to the PLSDB plasmid database (release 2018_12_05) (Galata et al., 2019) using dc-megablast (blastn 2.6.0+) (Altschul et al., 1990; Zhang et al., 2000) and identifying the best-hit alignment. Genes with sequence identity greater than 70% and coverage greater than 75% over the gene length were considered having homologs in known plasmids.

### Annotation of BGCs

BGCs were identified using antiSMASH (Weber et al., 2015) with default parameters.

### MIC Test of Selected Antibiotics and Isolates

Appropriate stock concentrations of selected antibiotics were prepared in TSB medium. Serial dilutions were made using TSB medium in a 96-well cell culture plate. Overnight cultures of selected S. epidermidis isolates were diluted in TSB medium and about ˜10⁵ cells were added to each well. The plate was incubated on a shaker at 37° C. for 18 hours and the growth of cells were determined by measuring the OD600.

### Annotation and Validation of agr Sequences

The agr genes (agrA, agrB, agrC, and agrD) were annotated by blasting all genes in the subject isolates (predicted using Prokka v1.11 (Seemann, 2014)) to the three canonical types of agr sequences as described in Olson et al. (2014). Specifically, the agr gene sequences annotated in strains NIHLM095 (GCF_000276545.1), NIHLM061 (GCF_000276445.1), and NIHLM037 (GCF_000276325.1), were used as reference sequences for agr type I, II, and III, respectively. An agrABCD operon was assigned to one of the three canonical agr types if 1) the AIP was identical to one of the three AIP types as described in Olson et al. (2014), and in the same time 2) the agrB and agrC genes had the highest sequence similarity to the same agr type as the AIP. The identified agr gene sequences were assigned to one of the three types based on the best match. The secondary structure of the AgrC protein was predicted using the Jpred 4 web server (Drozdetskiy et al., 2015) with default options.

Transmembrane mutations in agrC genes were validated in five selected isolates (isolate ID=644, 700, 1026, 1203, and 1523, which represented the five mutation patterns shown in FIG. 7B) using Sanger sequencing with primers S_epi_dupagrC_uni-F (CTGGAATTATAATCCTTTCTGC, forward, SEQ ID NO: 6) and S_epi_dupagrC_uni-R (GTAATCTGAAAGAGTGGTGAG, reverse, SEQ ID NO: 7) for all isolates except 0644 for which the forward primer was replaced with S_epi_dupagrC_66-F (TACGATTGTAATCCCTTCTGC, forward, SEQ ID NO: 8). Products of ˜640 bp were purified and Sanger-sequenced.

For Pacific Biosciences SMRT sequencing, genomic DNA was extracted using GenElute Bacterial Genomic DNA Kit (Sigma-Aldrich) from pelleted bacterial cells from 0.5 ml of overnight cultures with the addition of lysostaphin (Sigma-Aldrich) according to the manufacturer's protocol. DNA was sheared using a Megaruptor (Diagenode) to produce fragments with an average size of 6-8 kbp and further purified by binding to 0.45×AMPure beads. Sequencing libraries were prepared using SMRTbell Template Kit (PacBio) with barcoded SMRTbell adapters (PacBio). The resulting libraries were pooled for sequencing on a single SMRT cell on the Sequel system.

### Analyses of Quorum Sensing Interference

To determine the effect of mixture of agr types in natural populations on quorum sensing, six isolates of different agr types (isolate 71 and 73 for Type I, 72 and 74 for Type II, and 78 and 79 for Type III), found at the same skin site in the same subject (right index in p0), were chosen to simulate a isolate composition in a natural population. The six isolates were grown individually and the supernatant of these cultures were mixed to simulate naturally-occurring populations. To account for influences of the relative abundances of the isolates, the following population supernatants were created: 1) Evenly admixture supernatant: overnight culture from each of the six isolates was spun down, filter sterilized, and mixed in equal volume, and 2) population supernatant with the dominance of a single agr type: overnight culture from each of the six isolates was spun down, filter sterilized, and mixed such that the dominant agr type isolate supernatants composed 80% of the final volume and the supernatants from the remaining four isolates composed 20% of the final volume, equally). Next, the expression levels of ecp in three isolates representing three agr types (isolate 71 for Type I, 72 for Type II, and 78 for Type III) when exposed to self and the population supernatants were determined using RT-qPCR. Additionally, to illustrate the effect of agr type mixture on global gene expression, we performed RNA-seq on a randomly chosen isolate (isolate 71) grown in self supernatant, no supernatant, and evenly admixture supernatant. As controls, self-supernatants were diluted to the concentration of that agr type in the population mixture.

To investigate whether the newly identified Type IV agr can interfere with the quorum sensing of canonical agr types (Type I-III), agr isolates of Type I-III were grown separately either in the presence of Type IV spend media supernatant (from isolate 0644) or without the addition of any supernatant. Conversely, to test conditions that can potentially influence the quorum sensing of Type IV agr, an agr Type IV isolate (isolate 0644) was grown in the presence of Type I-III supernatant, without additional supernatant, or with self-supernatant, respectively. After the growth assays, the expression levels of ecp were determined using RT-qPCR.

Growth assays for all of the RT-qPCR and RNA-seq experiments were performed as following: One isolate of each agr Type I-IV (isolate 71, 72, 78, and 0644) was grown individually overnight, back diluted 1/100 in TSB, grown to an OD600 of ˜0.8, and back-diluted again to a starting OD600 of 0.05 in TSB with 10% supernatant by volume. No supernatant controls were grown in 100% fresh TSB. Sampling was performed at the start of the assay: aliquots were spun down, resuspended in Trizol, and froze at −80 C prior to RNA extraction for a zero-hour time point. The cultures were grown for four hours at 37 C and sampling was performed again, as described above. The experiment was performed with biological triplicates.

### RNA Extraction, RT-qPCR and RNA-Seq

Cultures were mechanically lysed in Trizol via bead-beating with 0.1 mm glass beads and RNA was isolated using a combination of Trizol/chloroform and on-column isolation using the Qiagen RNeasy Kit. Briefly, chloroform was added to the lysate, spun down, and the RNA in the organic layer was precipitated with 70% ethanol prior to washing (RW1 and RPE buffers, according to kit instructions) and elution on the RNeasy column. According to kit instructions, on-column DNAase was performed with the Qiagen RNase-free DNase kit. RNA concentration was measured via Qubit and the quality assessed via Agilent Tapestation. For RT-qPCR experiments, RNA was normalized and reverse transcribed into cDNA (Applied Biosystems High Capacity cDNA Reverse Transcription Kit). RT-qPCR was performed using the SYBR Power UP kit with ecp as the target gene and ftsZ as an internal control, according to kit instructions. For RNA-seq experiments, RNA was prepared for sequencing using the NEBNext rRNA Depletion Kit (Bacteria) (pre-release) and NEBNext Ultra II Directional RNA Library Prep Kit for Illumina kit according to kit instructions and sequenced on the Illumina NextSeq to a depth of 4-9.5 million reads.

### Comparison of Transcript Levels

For RT-qPCR, each growth assay was performed in biological triplicate in parallel. RT-qPCR results were analyzed using the comparative Ct analysis method (Schmittgen and Livak, 2008). First, Ct values of technical replicates (qPCR replicates from the same cDNA sample; n=2 for evenly mixed cultures and n=3 for uneven-mix experiments) were averaged. ddCt values were then calculated for each sample by subtracting the dCt of the zero-hour time point from the dCt value of the four-hour time point and relative quantification values were derived from the ddCt values (2^{-ddct}). Statistical significance was tested on the ddCt values using Welch's t-test.

For RNA-seq, the growth assay was performed in biological triplicate in parallel. Gene coding sequences of isolate 71 was first annotated using RAST, before sequencing reads were aligned to the gene sequences using Bowtie2 (v2.3.1) (Langmead and Salzberg, 2012; Langmead et al.) under very-sensitive mode. The output sam files were filtered to include only uniquely mapped reads (with the option “-q 1” in Samtools v1.8 (Li et al., 2009)), converted to bam files, sorted, and indexed using Samtools (v1.8) (Li et al., 2009). The raw count of reads aligned to each gene was computed using featureCounts (v1.5.2) (Liao et al., 2014) with default arguments. Differentially expressed genes were identified using the DESeq2 package (Love et al., 2014) (Benjamini-Hochberg adjusted p-value of <0.05) in R using the standard differential expression analysis workflow. Based on the DESeq2 results, the differentially abundant KEGG pathways were consequently inferred using the GAGE package (v2.28.2) (Luo et al., 2009) and visualized using the Pathview package (v1.18.2) (Luo and Brouwer, 2013).

mWGS Quality Filtering and Taxonomic Profiling

Sequencing adapters and low-quality bases were removed from the mWGS reads using scythe (v0.994) (Buffalo) and sickle (v1.33) (Joshi and Fass), respectively, with default parameters. Host reads were removed by mapping all sequencing reads to the hg19 human reference genome using Bowtie2 (v2.3.1) (Langmead and Salzberg, 2012; Langmead et al.), under “very-sensitive” mode. Unmapped reads (i.e., microbial reads) were used to estimate the relative abundance profiles of the microbial species in the samples using MetaPhlAn2 (Segata et al., 2012; Truong et al., 2015).

mWGS Assembly and Gene Prediction

Metagenomic genes were predicted from the mWGS samples using a method derived from (Zhou et al., 2019). mWGS reads from all skin microbiome samples were pooled and assembled de novo using MEGAHIT (v1.0.6) (Li et al., 2015, 2016) with default parameters. The resulting contigs were filtered by length (contigs no shorter than 1 kb were kept) before genes were predicted from the contigs using prodigal (v2.6.3) (Hyatt et al., 2010) under the “meta” mode. Predicted genes were clustered at 90% DNA sequence identity using UCLUST (the cluster_fast algorithm in USEARCH v8.0.1517, which sorts the gene sequences by length, conducts global alignments, and then trims terminal gaps before computing sequence identity (Edgar, 2010)) to remove redundant gene sequences. Next, the predicted metagenomic genes were blasted to the S. epidermidis pan-genome using UBLAST (USEARCH v8.0.1517 (Edgar, 2010)) with an e-value threshold of 10⁻⁹, and metagenomic genes with a DNA sequence identity >95% to any S. epidermidis genes were excluded, resulting in a catalog of 502,145 non-S. epidermidis metagenomic genes.

To estimate the coverage of the metagenomic genes in the microbiome samples, mWGS reads were mapped to the metagenomic genes using Bowtie2 (v2.3.1) (Langmead and Salzberg, 2012; Langmead et al.) and the coverage was computed using Samtools (v1.8, used for samfile to bamfile conversion and sorting) (Li et al., 2009) and Bedtools (v2.27.0, genomecov function) (Quinlan and Hall, 2010).

Linear Association Between Microbiome Features and S. epidermidis Gene Prevalence

Microbiome species with a mean relative abundance lower than 0.0001, and metagenomic genes with a mean coverage lower than 0.000001 reads per base per mWGS read sampled were excluded from downstream analyses. Next, microbiome species and genes were further filtered based on variability (excluded features with a coefficient of variation lower than 0.05) and sparsity (excluded features with non-zero abundance/coverage in more than 20% of the samples). Similarly, S. epidermidis genes that had a coefficient of variation lower than 0.05 or with non-zero abundance/coverage in more than 20% of the samples were not used for the analysis.

We then reduced the dimensionality of the microbiome species abundance profiles and the microbiome gene coverage profiles using principal component analyses (prcomp in R): the first 3 principal components that explained 82% of the variation in species abundance profiles, and the first 2 principal components that explained 90% of the variation in the gene coverage profiles were used to represent the microbiome species and gene compositions, respectively. Next, the influence of the microbiome species composition, or the microbiome gene coverage, on the prevalence of S. epidermidis gene content (i.e. the proportion of S. epidermidis isolates that carried the gene) were modeled separately, each using a linear model:

$$y = ( {\sum }_{i = 1}^{n} {a}_{i} P {C}_{i} ) + b P + c S + e T + d P \times S + ε$$

where y is the observed prevalence of a given gene, PCᵢ is the ith principal component (out of a n=3 principal components for microbiome species and n=2 principal components for microbiome genes), P denotes the subject, S denotes the skin site, T denotes the sampling time point, x denotes interaction effect, and ε is the residual error. The p-values (of the adjusted partial R² of the principal components) were estimated using unrestricted permutation, permutation restricted within-subject, and permutation restricted within subject×site, of the observed S. epidermidis gene prevalence before adjusted under the Benjamini-Hochberg procedure. Finally, S. epidermidis genes that were significant under all of the permutation tests were reported.
S. epidermidis Gene Prevalence Prediction Using a Recursive Partitioning Tree Model

We used a recursive partitioning tree model (implemented in the R package rpart v4.1.15 (Therneau and Atkinson, 2019)) to extract potentially non-linear relationships between microbiome/skin site features and S. epidermidis gene prevalence (FIG. 13E).

One limitation of our dataset is that only about 10 S. epidermidis isolates were sampled per skin site per subject, and thus the gene prevalence estimated based on this relatively small sample can approximate but may not accurately reflect the actual gene prevalence at the sample location. Therefore instead of training a regression tree to predict the numerical value of gene prevalence, we binned the gene prevalence into four levels (prevalent—gene prevalence ∈[0.75, 1], likely prevalent—gene prevalence ∈[0.5, 0.75), likely absent—gene prevalence ∈[0.25, 0.5), and absent—gene prevalence ∈[0, 0.25)) and trained a classification tree to predict the prevalence level. To balance the representation of prevalence levels, we only considered S. epidermidis genes that had exhibited all four prevalence levels across the samples.

Feature vectors were generated based on microbiome species abundances, microbiome gene coverages, and skin site specifications (FIG. 13E). The microbiome species and gene profiles were filtered based on abundance/coverage and variability as described in the previous section but were not screened based on sparsity as no significance tests were conducted. The microbiome gene profiles were then rescaled proportionally such that they share the same maximum and minimum values with the microbiome species profiles. Next, the microbiome species and gene profiles were combined before subjected to dimensionality reduction using principal component analyses (prcomp in R). For a given sample, we generated 15 feature vectors, each containing 1) the sampled skin site, and 2) the top x principal components (x=1, 2, . . . , 15), which explained 37%-90% of the variation in the microbiome features (FIG. 13E).

The dataset was divided into a training set (80% of the samples randomly chosen from p0, p1, p2, and p4), a validation set (the rest 20% of the samples from p0, p1, p2, and p4), and a test set (all samples from p3). For a given S. epidermidis gene, 15 recursive partitioning tree models were trained based on the 15 feature vectors, respectively, and evaluated based on their predictability—the probability of making correct prediction:

$$Predictability = {\sum }_{l = 1}^{4} {I}_{l} P {r}_{l}$$

where 1 indicates the four levels of prevalence, I₁ is an indicator variable which equals 1 if level 1 is the observed prevalence level and equals 0 otherwise. Pr₁ is the probability of level 1: for prior predictability, Pr₁ equals the observed frequency of level 1 in the training set; for posterior predictability, Pr₁ equals the “class probability” of level 1 given by the predict.rpart function. For a given S. epidermidis gene, the best model showing the highest posterior predictability based on the validation set was selected for downstream analysis. To separate the predictability due to skin site specification and the predictability due to the inclusion of microbiome data, we trained an additional set of recursive partitioning tree models with a similar approach but using only the skin site specification as the feature (that is, not including any microbiome features). Presence of homologs of S. epidermidis genes in known plasmids were estimated by aligning the genes to the PLSDB plasmid database (release 2018_12_05) (Galata et al., 2019) using dc-megablast (blastn 2.6.0+) (Altschul et al., 1990; Zhang et al., 2000) and identifying the best-hit alignment. Genes with sequence identity greater than 70% and coverage greater than 75% over the gene length were considered having homologs in known plasmids.

### Quantification and Statistical Analysis

Cophenetic distance was computed using the “cophenetic.phylo” function in the R package ape (v5.3) (Paradis and Schliep, 2019). Pielou's evenness index (J) was given by:

$$J = \frac{H}{ln S}$$

where H is the Shannon's diversity index computed using the R package vegan (v2.5.3) (Oksanen et al., 2018), and S is the total number of classes (in the case of the prevalence levels, S=4). FST was computed using:

$${F}_{S T} = \frac{{π}_{b e t w e e n} - {π}_{within}}{{π}_{b e t w e e n}}$$

where π_{between} and π_{within} respectively represent the average between-subpopulation and within-subpopulation pairwise gene content difference—the average proportion of genes that were present in only one isolate out of a pair of S. epidermidis isolates.

All significance tests, unless noted otherwise, were conducted in R (v3.2.3) with standard libraries. Hartigan's dip test was conducted using the R package diptest (v0.75.7) (Maechler, 2016) with p values estimated via the implemented linear interpolation method. PERMANOVA was conducted using the “adonis” function in the R package vegan (v2.5.3) (Oksanen et al., 2018), with subject, skin site, and their interaction term used as the covariates. Adjustment for false discovery rate was conducted following the Benjamini-Hochberg procedure (R function p.adjust with method=“BH”).

To test the underrepresentation of agr type III in p2 and type II in p4, we modeled the null hypothesis assuming that the sampling of agr types in the two subjects were Bernoullian, with the success rates given by the overall frequencies of the agr types in all 1482 subject isolates, and the number of trails given by the total number of S. epidermidis isolates sampled from the subjects. Therefore, the p-value can be given by the cumulative binomial distribution function:

$$P = {Σ}_{i = 0}^{k} ( \begin{matrix}n \\ i\end{matrix} ) {{f}^{i} ( 1 - f )}^{n - i}$$

where k is the observed number of S. epidermidis isolates in the subject with the agr type of interest, n is the total number of S. epidermidis isolates sampled from the subject, and f is the overall frequency of the agr type of interest in all 1482 subject isolates.

Permutation was implemented using a custom R script. Briefly, for linear models, a test statistics was first computed from an observation, before a total of at least 1000 permutations (unless noted otherwise) were generated by shuffling the dependent variable. The p-value was then expressed as the proportion of permutations yielding a larger test statistics than the observed test statistics. For ANOVA, the F statistics was used as the test statistics in the permutation. For generalized linear model, which was used to test association between microbiome features and S. epidermidis gene prevalence, the adjusted partial R² was used as the test statistics. For cases other than linear models, permutations were generated by re-distributing labels of the data. Specifically, to test the significance of temporal fluctuation in S. epidermidis gene content, permutations were generated by reassigning the time points among subject isolates sampled from the same skin site of the same subject (i.e. the same subpopulation), while the test statistics was given by the proportion of shared pan-genomes across time points.

### Data and Code Availability

The datasets generated during and/or analyzed during the current study, as well as custom codes to analyze the data, are available from the corresponding author on reasonable request. Genomes will be deposited in Genbank and metagenomic sequence reads in SRA under BioProject PRJNA559376 and PRJNA558989.

### REFERENCES

Altschul, S. F., Gish, W., Miller, W., Myers, E. W., and Lipman, D. J. (1990). Basic local alignment search tool. J. Mol. Biol. 215, 403-410.
Archer, G. L., and Johnston, J. L. (1983). Self-transmissible plasmids in staphylococci that encode resistance to aminoglycosides. Antimicrob. Agents and Chemother. 24, 70-77.
Arndt, D., Grant, J. R., Marcu, A., Sajed, T., Pon, A., Liang, Y., and Wishart, D. S. (2016). PHASTER: a better, faster version of the PHAST phage search tool. Nucleic Acids Res. 44, W16-21.
Asnicar, F., Manara, S., Zolfo, M., Truong, D. T., Scholz, M., Armanini, F., Ferretti, P., Gorfer, V., Pedrotti, A., Tett, A., et al. (2017). Studying Vertical Microbiome Transmission from Mothers to Infants by Strain-Level Metagenomic Profiling. MSystems 2, e00164-16.
Bankevich, A., Nurk, S., Antipov, D., Gurevich, A. A., Dvorkin, M., Kulikov, A. S., Lesin, V. M., Nikolenko, S. I., Pham, S., Prjibelski, A. D., et al. (2012). SPAdes: A New Genome Assembly Algorithm and Its Applications to Single-Cell Sequencing. J. Comput. Biol. 19, 455-477.
Batzilla, C. F., Rachid, S., Engelmann, S., Hecker, M., Hacker, J., and Ziebuhr, W. (2006). Impact of the accessory gene regulatory system (Agr) on extracellular proteins, codY expression and amino acid metabolism in Staphylococcus epidermidis. Proteomics 6, 3602-3613.
Bielejec, F., Baele, G., Vrancken, B., Suchard, M. A., Rambaut, A., and Lemey, P. (2016). SpreaD3: Interactive Visualization of Spatiotemporal History and Trait Evolutionary Processes. Mol. Biol. Evol. 33, 2167-2169.
Brisson, D. (2018). Negative Frequency-Dependent Selection Is Frequently Confounding. Front. Ecol. Evol. 6.
Brisson, D., and Dykhuizen, D. E. (2004). ospC diversity in Borrelia burgdorferi: different hosts are different niches. Genetics 168, 713-722.
Brito, I. L., and Alm, E. J. (2016). Tracking Strains in the Microbiome: Insights from Metagenomics and Models. Front. Microbiol. 7.
Buffalo, V. Scythe—A Bayesian adapter trimmer (https://github.com/vsbuffalo/scythe).
Chen, L., Zheng, D., Liu, B., Yang, J., and Jin, Q. (2016). VFDB 2016: hierarchical and refined dataset for big data analysis—10 years on. Nucleic Acids Res. 44, D694-697.
Cheung, G. Y. C., Joo, H.-S., Chatterjee, S. S., and Otto, M. (2014). Phenol-soluble modulins—critical determinants of staphylococcal virulence. FEMS Microbiol. Rev. 38, 698-719.
Cogen, A. L., Yamasaki, K., Sanchez, K. M., Dorschner, R. A., Lai, Y., MacLeod, D. T., Torpey, J. W., Otto, M., Nizet, V., Kim, J. E., et al. (2010a). Selective Antimicrobial Action Is Provided by Phenol-Soluble Modulins Derived from Staphylococcus epidermidis, a Normal Resident of the Skin. J. Invest. Dermatol. 130, 192-200.
Cogen, A. L., Yamasaki, K., Muto, J., Sanchez, K. M., Alexander, L. C., Tanios, J., Lai, Y., Kim, J. E., Nizet, V., and Gallo, R. L. (2010b). Staphylococcus epidermidis Antimicrobial 6-Toxin (Phenol-Soluble Modulin-7) Cooperates with Host Antimicrobial Peptides to Kill Group A Streptococcus. PLOS ONE 5, e8557.
Conlan, S., Mijares, L. A., Becker, J., Blakesley, R. W., Bouffard, G. G., Brooks, S., Coleman, H., Gupta, J., Gurson, N., Park, M., et al. (2012). Staphylococcus epidermidis pan-genome sequence analysis reveals diversity of skin commensal and hospital infection-associated isolates. Genome Biol. 13, R64.
Dalkiran, A., Rifaioglu, A. S., Martin, M. J., Cetin-Atalay, R., Atalay, V., and Dogan, T. (2018). ECPred: a tool for the prediction of the enzymatic functions of protein sequences based on the EC nomenclature. BMC Bioinformatics 19, 334.
Didelot, X., and Wilson, D. J. (2015). ClonalFrameML: Efficient Inference of Recombination in Whole Bacterial Genomes. PLOS Comput. Biol. 11, e1004041.
Dobzhansky, T. (1982). Genetics and the Origin of Species: Columbia Classics edition (Columbia University Press).
Drozdetskiy, A., Cole, C., Procter, J., and Barton, G. J. (2015). JPred4: a protein secondary structure prediction server. Nucleic Acids Res. 43, W389-W394.
Drummond, A. J., Rambaut, A., Shapiro, B., and Pybus, O. G. (2005). Bayesian coalescent inference of past population dynamics from molecular sequences. Mol. Biol. Evol. 22, 1185-1192.
Duchêne, S., Holt, K. E., Weill, F.-X., Le Hello, S., Hawkey, J., Edwards, D. J., Fourment, M., and Holmes, E. C. (2016). Genome-scale rates of evolutionary change in bacteria. Microb. Genom. 2, e000094.
Duvallet, C., Gibbons, S. M., Gurry, T., Irizarry, R. A., and Alm, E. J. (2017). Meta-analysis of gut microbiome studies identifies disease-specific and shared responses. Nat. Commun. 8, 1784. Edgar, R. C. (2010). Search and clustering orders of magnitude faster than BLAST. Bioinformatics 26, 2460-2461.
von Eiff, C., Becker, K., Machka, K., Stammer, H., and Peters, G. (2001). Nasal carriage as a source of Staphylococcus aureus bacteremia. Study Group. N. Engl. J. Med. 344, 11-16. El-Gebali, S., Mistry, J., Bateman, A., Eddy, S. R., Luciani, A., Potter, S. C., Qureshi, M., Richardson, L. J., Salazar, G. A., Smart, A., et al. (2019). The Pfam protein families database in 2019. Nucleic Acids Res. 47, D427-D432.
Ferretti, P., Pasolli, E., Tett, A., Asnicar, F., Gorfer, V., Fedi, S., Armanini, F., Truong, D. T., Manara, S., Zolfo, M., et al. (2018). Mother-to-Infant Microbial Transmission from Different Body Sites Shapes the Developing Infant Gut Microbiome. Cell Host Microbe 24, 133-145.
Fey, P. D., and Olson, M. E. (2010). Current concepts in biofilm formation of Staphylococcus epidermidis. Future Microbiol. 5, 917-933.
Forbes, B. A., and Schaberg, D. R. (1983). Transfer of resistance plasmids from Staphylococcus epidermidis to Staphylococcus aureus: evidence for conjugative exchange of resistance. J. Bacteriol. 153, 627-634.
Frisch, M. B., Castillo-Ramirez, S., Petit, R. A., Farley, M. M., Ray, S. M., Albrecht, V. S., Limbago, B. M., Hernandez, J., See, I., Satola, S. W., et al. (2018). Invasive Methicillin-Resistant Staphylococcus aureus USA500 Strains from the U.S. Emerging Infections Program Constitute Three Geographically Distinct Lineages. MSphere 3, e00571-17.
Fu, L., Niu, B., Zhu, Z., Wu, S., and Li, W. (2012). CD-HIT: accelerated for clustering the next-generation sequencing data. Bioinformatics 28, 3150-3152.
Galata, V., Fehlmann, T., Backes, C., and Keller, A. (2019). PLSDB: a resource of complete bacterial plasmids. Nucleic Acids Res. 47, D195-D202.
Greenblum, S., Carr, R., and Borenstein, E. (2015). Extensive Strain-Level Copy-Number Variation across Human Gut Microbiome Species. Cell 160, 583-594.
Gurevich, A., Saveliev, V., Vyahhi, N., and Tesler, G. (2013). QUAST: quality assessment tool for genome assemblies. Bioinformatics 29, 1072-1075.
Hedge, J., and Wilson, D. J. (2014). Bacterial Phylogenetic Reconstruction from Whole Genomes Is Robust to Recombination but Demographic Inference Is Not. MBio 5, e02158-14.
Hirvonen, J. J., Kerttula, A.-M., and Kaukoranta, S.-S. (2014). Performance of SaSelect, a Chromogenic Medium for Detection of Staphylococci in Clinical Specimens. J. Clin. Microbiol. 52, 1041-1044.
Huerta-Cepas, J., Szklarczyk, D., Forslund, K., Cook, H., Heller, D., Walter, M. C., Rattei, T., Mende, D. R., Sunagawa, S., Kuhn, M., et al. (2016). eggNOG 4.5: a hierarchical orthology framework with improved functional annotations for eukaryotic, prokaryotic and viral sequences. Nucleic Acids Res. 44, D286-D293.
Hyatt, D., Chen, G.-L., LoCascio, P. F., Land, M. L., Larimer, F. W., and Hauser, L. J. (2010). Prodigal: prokaryotic gene recognition and translation initiation site identification. BMC Bioinformatics 11, 119.
Jia, B., Raphenya, A. R., Alcock, B., Waglechner, N., Guo, P., Tsang, K. K., Lago, B. A., Dave, B. M., Pereira, S., Sharma, A. N., et al. (2017). CARD 2017: expansion and model-centric curation of the comprehensive antibiotic resistance database. Nucleic Acids Res. 45, D566-D573.
Joshi, N. A., and Fass, J. N. Sickle: A sliding-window, adaptive, quality-based trimming tool for FastQ files (https://github.com/najoshi/sickle).
Kong, H. H., Oh, J., Deming, C., Conlan, S., Grice, E. A., Beatson, M. A., Nomicos, E., Polley, E. C., Komarow, H. D., Program, N. C. S., et al. (2012). Temporal shifts in the skin microbiome associated with disease flares and treatment in children with atopic dermatitis. Genome Res. 22, 850-859.
Krawczyk, P. S., Lipinski, L., and Dziembowski, A. (2018). PlasFlow: predicting plasmid sequences in metagenomic data using genome signatures. Nucleic Acids Res. 46, e35.
Kurtz, S., Phillippy, A., Delcher, A. L., Smoot, M., Shumway, M., Antonescu, C., and Salzberg, S. L. (2004). Versatile and open software for comparing large genomes. Genome Biol. 5, R12.
Lai, Y., Nardo, A. D., Nakatsuji, T., Leichtle, A., Yang, Y., Cogen, A. L., Wu, Z.-R., Hooper, L. V., Schmidt, R. R., Aulock, S. von, et al. (2009). Commensal bacteria regulate Toll-like receptor 3-dependent inflammation after skin injury. Nat. Med. 15, 1377-1382.
Lai, Y., Cogen, A. L., Radek, K. A., Park, H. J., MacLeod, D. T., Leichtle, A., Ryan, A. F., Di Nardo, A., and Gallo, R. L. (2010). Activation of TLR2 by a Small Molecule Produced by Staphylococcus epidermidis Increases Antimicrobial Defense against Bacterial Skin Infections. J. Invest. Dermatol. 130, 2211-2221.
Lam, H. M., Ratmann, O., and Boni, M. F. (2018). Improved Algorithmic Complexity for the 3SEQ Recombination Detection Algorithm. Mol. Biol. Evol. 35, 247-251.
Langfelder, P., Zhang, B., and Horvath, S. (2008). Defining clusters from a hierarchical cluster tree: the Dynamic Tree Cut package for R. Bioinformatics 24, 719-720.
Langmead, B., and Salzberg, S. L. (2012). Fast gapped-read alignment with Bowtie 2. Nat. Methods 9, 357-359.
Langmead, B., Wilks, C., Antonescu, V., Charles, R., and Hancock, J. Scaling read aligners to hundreds of threads on general-purpose processors. Bioinformatics.
Le, K. Y., and Otto, M. (2015). Quorum-sensing regulation in staphylococci—an overview. Front. Microbiol. 6.
Leimbach, A., Hacker, J., and Dobrindt, U. (2013). E. coli as an all-rounder: the thin line between commensalism and pathogenicity. Curr. Top. Microbiol. Immunol. 358, 3-32.
Lemey, P., Rambaut, A., Drummond, A. J., and Suchard, M. A. (2009). Bayesian Phylogeography Finds Its Roots. PLOS Comput. Biol. 5, e1000520.
Li, H. (2011). A statistical framework for SNP calling, mutation discovery, association mapping and population genetical parameter estimation from sequencing data. Bioinformatics 27, 2987-2993.
Li, W., and Godzik, A. (2006). Cd-hit: a fast program for clustering and comparing large sets of protein or nucleotide sequences. Bioinformatics 22, 1658-1659.
Li, D., Liu, C.-M., Luo, R., Sadakane, K., and Lam, T.-W. (2015). MEGAHIT: an ultra-fast single-node solution for large and complex metagenomics assembly via succinct de Bruijn graph. Bioinformatics 31, 1674-1676.
Li, D., Luo, R., Liu, C.-M., Leung, C.-M., Ting, H.-F., Sadakane, K., Yamashita, H., and Lam, T.-W. (2016). MEGAHIT v1.0: A fast and scalable metagenome assembler driven by advanced methodologies and community practices. Methods 102, 3-11.
Li, H., Handsaker, B., Wysoker, A., Fennell, T., Ruan, J., Homer, N., Marth, G., Abecasis, G., Durbin, R., and 1000 Genome Project Data Processing Subgroup (2009). The Sequence Alignment/Map format and SAMtools. Bioinformatics 25, 2078-2079.
Liao, Y., Smyth, G. K., and Shi, W. (2014). featureCounts: an efficient general purpose program for assigning sequence reads to genomic features. Bioinformatics 30, 923-930.
Linehan, J. L., Harrison, O. J., Han, S.-J., Byrd, A. L., Vujkovic-Cvijin, I., Villarino, A. V., Sen, S. K., Shaik, J., Smelkinson, M., Tamoutounour, S., et al. (2018). Non-classical immunity controls microbiota impact on skin immunity and tissue repair. Cell 172, 784-796.e18.
Lloyd-Price, J., Mahurkar, A., Rahnavard, G., Crabtree, J., Orvis, J., Hall, A. B., Brady, A., Creasy, H. H., McCracken, C., Giglio, M. G., et al. (2017). Strains, functions and dynamics in the expanded Human Microbiome Project. Nature 550, 61-66.
Love, M. I., Huber, W., and Anders, S. (2014). Moderated estimation of fold change and dispersion for RNA-seq data with DESeq2. Genome Biol. 15.
Luo, W., and Brouwer, C. (2013). Pathview: an R/Bioconductor package for pathway-based data integration and visualization. Bioinformatics 29, 1830-1831.
Luo, W., Friedman, M. S., Shedden, K., Hankenson, K. D., and Woolf, P. J. (2009). GAGE: generally applicable gene set enrichment for pathway analysis. BMC Bioinformatics 10, 161.
MacLea, K. S., and Trachtenberg, A. M. (2017). Complete Genome Sequence of Staphylococcus epidermidis ATCC 12228 Chromosome and Plasmids, Generated by Long-Read Sequencing. Genome Announc. 5.
Maechler, M. (2016). diptest: Hartigan's Dip Test Statistic for Unimodality—Corrected. Martin, D., and Rybicki, E. (2000). RDP: detection of recombination amongst aligned sequences. Bioinformatics 16, 562-563.
Martin, D. P., Posada, D., Crandall, K. A., and Williamson, C. (2005). A modified bootscan algorithm for automated identification of recombinant sequences and recombination breakpoints. AIDS Res. Hum. Retroviruses 21, 98-102.
Martin, D. P., Murrell, B., Golden, M., Khoosal, A., and Muhire, B. (2015). RDP4: Detection and analysis of recombination patterns in virus genomes. Virus Evol. 1, vev003.
Matinaho, S., von Bonsdorff, L., Rouhiainen, A., Lönnroth, M., and Parkkinen, J. (2001). Dependence of Staphylococcus epidermidis on non-transferrin-bound iron for growth. FEMS Microbiol. Lett. 196, 177-182.
Méric, G., Miragaia, M., de Been, M., Yahara, K., Pascoe, B., Mageiros, L., Mikhail, J., Harris, L. G., Wilkinson, T. S., Rolo, J., et al. (2015). Ecological Overlap and Horizontal Gene Transfer in Staphylococcus aureus and Staphylococcus epidermidis. Genome Biol. Evol. 7, 1313-1328.
Méric, G., Mageiros, L., Pensar, J., Laabei, M., Yahara, K., Pascoe, B., Kittiwan, N., Tadee, P., Post, V., Lamble, S., et al. (2018). Disease-associated genotypes of the commensal skin bacterium Staphylococcus epidermidis. Nat. Commun. 9, 5034.
Mideo, N., Alizon, S., and Day, T. (2008). Linking within- and between-host dynamics in the evolutionary epidemiology of infectious diseases. Trends Ecol. Evol. 23, 511-517.
Naik, S., Bouladoux, N., Wilhelm, C., Molloy, M. J., Salcedo, R., Kastenmuller, W., Deming, C., Quinones, M., Koo, L., Conlan, S., et al. (2012). Compartmentalized Control of Skin Immunity by Resident Commensals. Science 337, 1115-1119.
Nakatsuji, T., Chen, T. H., Butcher, A. M., Trzoss, L. L., Nam, S.-J., Shirakawa, K. T., Zhou, W., Oh, J., Otto, M., Fenical, W., et al. (2018). A commensal strain of Staphylococcus epidermidis protects against skin neoplasia. Sci. Adv. 4, eaao4502.
Nataro, J. P., and Kaper, J. B. (1998). Diarrheagenic Escherichia coli. Clin. Microbiol. Rev. 11, 142-201.
National Nosocomial Infections Surveillance System (2004). National Nosocomial Infections Surveillance (NNIS) System Report, data summary from January 1992 through June 2004, issued October 2004. Am. J. Infect. Control. 32, 470-485.
Niehus, R., Mitri, S., Fletcher, A. G., and Foster, K. R. (2015). Migration and horizontal gene transfer divide microbial genomes into multiple niches. Nat. Commun. 6, 8924.
O'Brien, J. D., Didelot, X., Iqbal, Z., Amenga-Etego, L., Ahiska, B., and Falush, D. (2014). A Bayesian Approach to Inferring the Phylogenetic Structure of Communities from Metagenomic Data. Genetics 197, 925-937.
Oh, J., Byrd, A. L., Deming, C., Conlan, S., Kong, H. H., and Segre, J. A. (2014). Biogeography and individuality shape function in the human skin metagenome. Nature 514, 59-64.
Oh, J., Byrd, A. L., Park, M., Kong, H. H., and Segre, J. A. (2016). Temporal Stability of the Human Skin Microbiome. Cell 165, 854-866.
Oksanen, J., BLanchet, F. G., Friendly, M., Roeland, K., Legendre, P., McGlinn, D., Minchin, P.,
O'Hara, R. B., Simpson, G. L., Solymos, P., et al. (2018). vegan: Community Ecology Package.
Oliveira, F., Franga, A., and Cerca, N. (2017). Staphylococcus epidermidis is largely dependent on iron availability to form biofilms. Int. J. Med. Microbiol. 307, 552-563.
Olson, M. E., Todd, D. A., Schaeffer, C. R., Paharik, A. E., Dyke, M. J. V., Buttner, H., Dunman,
P. M., Rohde, H., Cech, N. B., Fey, P. D., et al. (2014). Staphylococcus epidermidis agr Quorum-Sensing System: Signal Identification, Cross Talk, and Importance in Colonization. J. Bacteriol. 196, 3482-3493.
Otto, M. (2009). Staphylococcus epidermidis—the “accidental” pathogen. Nat. Rev. Microbiol. 7, 555-567.
Otto, M., O'Mahoney, D. S., Guina, T., and Klebanoff, S. J. (2004). Activity of Staphylococcus epidermidis phenol-soluble modulin peptides expressed in Staphylococcus carnosus. J. Infect. Dis. 190, 748-755.
Padidam, M., Sawyer, S., and Fauquet, C. M. (1999). Possible emergence of new geminiviruses by frequent recombination. Virology 265, 218-225.
Page, A. J., Cummins, C. A., Hunt, M., Wong, V. K., Reuter, S., Holden, M. T. G., Fookes, M., Falush, D., Keane, J. A., and Parkhill, J. (2015). Roary: rapid large-scale prokaryote pan genome analysis. Bioinformatics 31, 3691-3693.
Paradis, E., and Schliep, K. (2019). ape 5.0: an environment for modern phylogenetics and evolutionary analyses in R. Bioinformatics 35, 526-528.
Parks, D. H., Imelfort, M., Skennerton, C. T., Hugenholtz, P., and Tyson, G. W. (2015). CheckM: assessing the quality of microbial genomes recovered from isolates, single cells, and metagenomes. Genome Res. 25, 1043-1055.
Posada, D., and Crandall, K. A. (2001). Evaluation of methods for detecting recombination from DNA sequences: computer simulations. Proc. Natl. Acad. Sci. U.S.A. 98, 13757-13762.
Potter, S. C., Luciani, A., Eddy, S. R., Park, Y., Lopez, R., and Finn, R. D. (2018). HMMER web server: 2018 update. Nucleic Acids Res. 46, W200-W204.
Queck, S. Y., Jameson-Lee, M., Villaruz, A. E., Bach, T.-H. L., Khan, B. A., Sturdevant, D. E., Ricklefs, S. M., Li, M., and Otto, M. (2008). RNAIII-independent target gene control by the agr quorum-sensing system: insight into the evolution of virulence regulation in Staphylococcus aureus. Mol. Cell 32, 150-158.
Quince, C., Delmont, T. O., Raguideau, S., Alneberg, J., Darling, A. E., Collins, G., and Eren, A. M. (2017). DESMAN: a new tool for de novo extraction of strains from metagenomes. Genome Biol. 18, 181.
Quinlan, A. R., and Hall, I. M. (2010). BEDTools: a flexible suite of utilities for comparing genomic features. Bioinformatics 26, 841-842.
Rambaut, A. FigTree version 1.3.1 (http://tree.bio.ed.ac.uk/).
Sakr, A., Brdgeon, F., Mege, J.-L., Rolain, J.-M., and Blin, O. (2018). Staphylococcus aureus Nasal Colonization: An Update on Mechanisms, Epidemiology, Risk Factors, and Subsequent Infections. Front. Microbiol. 9.
Scharschmidt, T. C., Vasquez, K. S., Truong, H.-A., Gearty, S. V., Pauli, M. L., Nosbaum, A., Gratz, I. K., Otto, M., Moon, J. J., Liese, J., et al. (2015). A wave of regulatory T cells into neonatal skin mediates tolerance to commensal microbes. Immunity 43, 1011-1021.
Schmittgen, T. D., and Livak, K. J. (2008). Analyzing real-time PCR data by the comparative CT method. Nat. Protoc. 3, 1101-1108.
Seemann, T. (2014). Prokka: rapid prokaryotic genome annotation. Bioinformatics 30, 2068-2069.
Segata, N. (2018). On the Road to Strain-Resolved Comparative Metagenomics. MSystems 3, e00190-17.
Segata, N., Waldron, L., Ballarini, A., Narasimhan, V., Jousson, O., and Huttenhower, C. (2012). Metagenomic microbial community profiling using unique clade-specific marker genes. Nat. Methods 9, 811-814.
Smillie, C. S., Sauk, J., Gevers, D., Friedman, J., Sung, J., Youngster, I., Hohmann, E. L., Staley, C., Khoruts, A., Sadowsky, M. J., et al. (2018). Strain Tracking Reveals the Determinants of Bacterial Engraftment in the Human Gut Following Fecal Microbiota Transplantation. Cell Host Microbe 23, 229-240.e5.
Smith, J. M. (1992). Analyzing the mosaic structure of genes. J. Mol. Evol. 34, 126-129.
Stamatakis, A. (2014). RAxML version 8: a tool for phylogenetic analysis and post-analysis of large phylogenies. Bioinformatics 30, 1312-1313.
Suchard, M. A., Lemey, P., Baele, G., Ayres, D. L., Drummond, A. J., and Rambaut, A. (2018). Bayesian phylogenetic and phylodynamic data integration using BEAST 1.10. Virus Evol. 4.
Tett, A., Pasolli, E., Farina, S., Truong, D. T., Asnicar, F., Zolfo, M., Beghini, F., Armanini, F., Jousson, O., Sanctis, V. D., et al. (2017). Unexplored diversity and strain-level structure of the skin microbiome associated with psoriasis. Npj Biofilms Microbiomes 3, 14.
Therneau, T., and Atkinson, B. (2019). rpart: Recursive Partitioning and Regression Trees. R package version 4.1-15.
Treangen, T. J., Ondov, B. D., Koren, S., and Phillippy, A. M. (2014). The Harvest suite for rapid core-genome alignment and visualization of thousands of intraspecific microbial genomes. Genome Biol. 15, 524.
Trivier, D., and Courcol, R. J. (1996). Iron depletion and virulence in Staphylococcus aureus. FEMS Microbiol. Lett. 141, 117-127.
Trivier, D., Davril, M., Houdret, N., and Courcol, R. J. (1995). Influence of iron depletion on growth kinetics, siderophore production, and protein expression of Staphylococcus aureus. FEMS Microbiol. Lett. 127, 195-199.
Truong, D. T., Franzosa, E. A., Tickle, T. L., Scholz, M., Weingart, G., Pasolli, E., Tett, A., Huttenhower, C., and Segata, N. (2015). MetaPhlAn2 for enhanced metagenomic taxonomic profiling. Nat. Methods 12, 902-903.
Truong, D. T., Tett, A., Pasolli, E., Huttenhower, C., and Segata, N. (2017). Microbial strain-level population structure and genetic diversity from metagenomes. Genome Res. 27, 626-638.
Wang, L., and Archer, G. L. (2010). Roles of CcrA and CcrB in Excision and Integration of Staphylococcal Cassette Chromosome mec, a Staphylococcus aureus Genomic Island. J. Bacteriol. 192, 3204-3212.
Wang, R., Khan, B. A., Cheung, G. Y. C., Bach, T.-H. L., Jameson-Lee, M., Kong, K.-F., Queck, S. Y., and Otto, M. (2011). Staphylococcus epidermidis surfactant peptides promote biofilm maturation and dissemination of biofilm-associated infection in mice. J. Clin. Invest. 121, 238-248.
Weber, T., Blin, K., Duddela, S., Krug, D., Kim, H. U., Bruccoleri, R., Lee, S. Y., Fischbach, M. A., Müller, R., Wohlleben, W., et al. (2015). antiSMASH 3.0—a comprehensive resource for the genome mining of biosynthetic gene clusters. Nucleic Acids Res. 43, W237-W243.
Wood, D. E., and Salzberg, S. L. (2014). Kraken: ultrafast metagenomic sequence classification using exact alignments. Genome Biol. 15, R46.
Yamada, M., Yoshida, J., Hatou, S., Yoshida, T., and Minagawa, Y. (2008). Mutations in the quinolone resistance determining region in Staphylococcus epidermidis recovered from conjunctiva and their association with susceptibility to various fluoroquinolones. Br. J. Ophthalmol. 92, 848-851.
Yao, Y., Vuong, C., Kocianova, S., Villaruz, A. E., Lai, Y., Sturdevant, D. E., and Otto, M. (2006). Characterization of the Staphylococcus epidermidis Accessory-Gene Regulator Response: Quorum-Sensing Regulation of Resistance to Human Innate Host Defense. J. Infect. Dis. 193, 841-848.
Yarwood, J. M., and Schlievert, P. M. (2003). Quorum sensing in Staphylococcus infections. J. Clin. Invest. 112, 1620-1625.
Yassour, M., Jason, E., Hogstrom, L. J., Arthur, T. D., Tripathi, S., Siljander, H., Selvenius, J., Oikarinen, S., Hyöty, H., Virtanen, S. M., et al. (2018). Strain-Level Analysis of Mother-to-Child Bacterial Transmission during the First Few Months of Life. Cell Host Microbe 24, 146-154.
Zhang, C., and Zhao, L. (2016). Strain-level dissection of the contribution of the gut microbiome to human metabolic disease. Genome Med. 8, 41.
Zhang, H., Ishige, K., and Kornberg, A. (2002). A polyphosphate kinase (PPK2) widely conserved in bacteria. Proc. Natl. Acad. Sci. U.S.A. 99, 16678-16683.
Zhang, Y.-Q., Ren, S.-X., Li, H.-L., Wang, Y.-X., Fu, G., Yang, J., Qin, Z.-Q., Miao, Y.-G., Wang, W.-Y., Chen, R.-S., et al. (2003). Genome-based analysis of virulence genes in a non-biofilm-forming Staphylococcus epidermidis strain (ATCC 12228). Mol. Microbiol. 49, 1577-1593.
Zhang, Z., Schwartz, S., Wagner, L., and Miller, W. (2000). A Greedy Algorithm for Aligning DNA Sequences. J. Comput. Biol. 7, 203-214.
Zhao, S., Lieberman, T. D., Poyet, M., Kauffman, K. M., Gibbons, S. M., Groussin, M., Xavier, R. J., and Alm, E. J. (2019). Adaptive Evolution within Gut Microbiomes of Healthy People. Cell Host Microbe 25, 656-667.e8.
Zhou, W., Chow, K., Fleming, E., and Oh, J. (2019). Selective colonization ability of human fecal microbes in different mouse gut environments. ISME J. 13, 805-823.
Zmora, N., Zilberman-Schapira, G., Suez, J., Mor, U., Dori-Bachash, M., Bashiardes, S., Kotler, E., Zur, M., Regev-Lehavi, D., Brik, R. B.-Z., et al. (2018). Personalized Gut Mucosal Colonization Resistance to Empiric Probiotics Is Associated with Unique Host and Microbiome Features. Cell 174, 1388-1405.
Zock, J., Cantwell, C., Swartling, J., Hodges, R., Pohl, T., Sutton, K., Rosteck, P., McGilvray, D., and Queener, S. (1994). The Bacillus subtilis pnbA gene encoding p-nitrobenzyl esterase: cloning, sequence and high-level expression in Escherichia coli. Gene 151, 37-43.

All references, patents and patent applications disclosed herein are incorporated by reference with respect to the subject matter for which each is cited, which in some cases may encompass the entirety of the document.

The indefinite articles “a” and “an,” as used herein the specification and in the claims, unless clearly indicated to the contrary, should be understood to mean “at least one.”

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

The terms “about” and “substantially” preceding a numerical value mean±10% of the recited numerical value.

Where a range of values is provided, each value between the upper and lower ends of the range are specifically contemplated and described herein.

1. A composition comprising (a) an admixture of cells that comprises at least two bacterial cell types selected from Staphylococcus (S.) epidermidis Type I cells, S. epidermidis Type II cells, S. epidermidis Type III cells, S. epidermidis Type IIIb cells, and S. epidermidis Type IV cells; and (b) a synthetic excipient.

2. The composition of claim 1, wherein the synthetic excipient is selected from the group consisting of diluents, thickeners, humectants, preservatives, neutralizers, coemulsifiers, occlusive, and fragrances.

3. A composition comprising an admixture of cells that comprises at least two bacterial cell types selected from Staphylococcus (S.) epidermidis Type I cells, S. epidermidis Type II cells, S. epidermidis Type III cells, S. epidermidis Type IIIb cells, and S. epidermidis Type IV cells, wherein at least one of the S. epidermidis cell types is genetically engineered.

4. An admixture of cells that comprises at least two cell types selected from Staphylococcus (S.) epidermidis Type I cells, S. epidermidis Type II cells, S. epidermidis Type III cells, S. epidermidis Type IIIb cells, and S. epidermidis Type IV cells, wherein the admixture is formulated with an excipient for topical delivery.

5. The composition or admixture of any one of the preceding claims, wherein cells of the admixture are collectively capable of modifying an expression level of a gene selected from nitrogen respiration genes, urease activity genes, carbohydrate metabolism genes, iron uptake genes, sulfur metabolism genes, and virulence factor genes, relative to a control.

6. The composition or admixture of claim 5, wherein the nitrogen respiration genes are selected from S. epidermidis narT, nreC, nreB, narX, narH, narG, sumT, nasE, and nasD.

7. The composition or admixture of claim 5 or 6, wherein the urease activity genes are selected from S. epidermidis ureA, ureB, ureC, ureD, ureE, and ureF.

8. The composition or admixture of any one of claims 5-7, wherein the carbohydrate metabolism genes are selected from S. epidermidis FruB, GlmS, Fba, Tkt, Tpi, Gap, GapB, Pgi, Pgk, Eno, PepcK, CitB, CitC, OgdH, Sucla2, SdhA, CitG, Mdh1, PckA, LDH, PDH, ScaD, PflB, Fdh, and BdhA.

9. The composition or admixture of any one of claims 5-8, wherein the iron uptake genes are selected from S. epidermidis fecD, feuC, fecE, and yclQ.

10. The composition or admixture of any one of claims 5-9, wherein the sulfur metabolism genes are selected from S. epidermidis cysH, cysJ, cysI, sirC, sat, and cysC.

11. The composition or admixture of any one of claims 5-10, wherein the virulence factor genes are selected from S. epidermidis icaA, icaB, icaC, icaD, icaR, atl, atlE, ebh, ebhA, ebp, geh, gehD, hlb, lip, nuc, psmB, sdrF, sdrH, se2319, sspA, and sspB.

12. The composition or admixture of any one of the preceding claims, wherein the expression level of the gene is decreased by between 1.5-fold and 12-fold.

13. The composition or admixture of any one of the preceding claims, wherein at least one of the Staphylococcus epidermidis cell types is genetically engineered.

14. A method of treating or preventing skin disease in a subject comprising administering to the subject the composition or admixture of any one of the preceding claims.

15. The method of claim 14, wherein the skin disease is selected from folliculitis, furuncles, carbuncles, impetigo, ecthyma, cellulitis, atopic dermatitis, scabies, folliculitis decalvans, and Netherton syndrome.

16. A method of reducing the risk of skin disease in a subject comprising administering to the subject the admixture of any one of the preceding claims.

17. The method of claim 16, wherein the skin disease is a recurrent skin disease.

18. The method of claim 17, wherein the recurrent disease is atopic dermatitis.

19. The method of claim 17, wherein the skin disease is selected from folliculitis, furuncles, carbuncles, impetigo, ecthyma, cellulitis, atopic dermatitis, scabies, folliculitis decalvans, and Netherton syndrome.

20. The method of any one of claims 14-19, wherein the administration is topical administration.

21. A method of treating or preventing bloodstream infection in a subject comprising administering to the subject the composition or admixture of any one of the preceding claims.


SEQUENCE LISTING (8 sequences)

SEQ ID NO: 1 - protein, 46 aa, Staphylococcus epidermidis
MENIFNLFIKFFTTILEFIGTVAGDSVCASYFDEPEVPEELTKLYE

SEQ ID NO: 2 - protein, 46 aa, Staphylococcus epidermidis
MNLLGGLLLKIFSNFMAVIGNASKYNPCSNYLDEPQVPEELTKLDE

SEQ ID NO: 3 - protein, 46 aa, Staphylococcus epidermidis
MNLLGGLLLKLFSNFMAVIGNAAKYNPCASYLDEPQVPEELTKLDE

SEQ ID NO: 4 - protein, 46 aa, Staphylococcus epidermidis
MNLLGGLFLKIFSNFMAVIGNAAKYNPCASYLDEPQVPEELTKLDE

SEQ ID NO: 5 - protein, 46 aa, Staphylococcus epidermidis
MNLLGGLLLKIFSNFMAVIGNASKYNPCVMYLDEPQVPEELTKLDE

SEQ ID NO: 6 - DNA, 22 bp, Artificial Sequence
  Synthetic
ctggaattataatcctttctgc

SEQ ID NO: 7 - DNA, 21 bp, Artificial Sequence
  Synthetic
gtaatctgaaagagtggtgag

SEQ ID NO: 8 - DNA, 21 bp, Artificial Sequence
  Synthetic
tacgattgtaatcccttctgc